SEQUENCE LISTING (48 sequences)

SEQ ID NO: 1 - DNA, 1992 bp, synthetic construct
  source 1..1992: mol_type = other DNA
atgttcgtgttcctggtcctgctcccactggtgtcctcgcagtgcgtgaacctgaccacccgcacccagctgccccctgcgtacaccaactccatcaccaggggcgtctactatcccgacaaggtgtttcgaagttccgttctgcactcgacccaagacctgttcctccccttcttcagcaacgtgacctggttccatgctatccacgtatcgggtacgaacgggaccaagcgcttcgacaaccccgtcctgcccttcaacgacggcgtgtactttgcatccaccgagaagtcgaacattatcagggggtggattttcggcaccaccctagactcgaagacacagtcgctgctgatcgtgaacaacgcgaccaatgtcgtcatcaaggtttgcgaattccagttctgcaatgaccccttcctgggagtctactaccacaagaacaacaagagctggatggagagtgagttcagagtgtattcctccgctaacaattgcaccttcgagtacgtatcccagcccttcctgatggacctggagggcaagcaggggaactttaaaaatctgcgcgagttcgtgtttaagaacatcgacggctacttcaagatctatagtaagcacacacccataaatctggtccgcgacctgccgcaaggcttctccgctttggaacccctcgtggacctgccgattgggatcaacatcacacgttttcagactctcctggccctgcaccggagttacctgacacccggtgactcctcctcaggctggactgccggggccgccgcttactacgtcggctatttgcagcccagaactttcctcctgaagtataacgagaacggcacgatcaccgatgccgtggattgtgcgctggacccgctgtcggagacaaagtgcacgctgaagtcgtttaccgtggagaaggggatctaccagacatctaacttccgggttcagcccactgagtccatcgtgcgcttccccaacatcacgaatctctgccccttcggcgaggtgttcaacgcaactcgcttcgccagtgtgtacgcctggaaccgcaagcggatttccaattgtgtcgctgactacagtgtgctttacaactctgcctcattcagtacgttcaagtgttacggcgtgtccccgaccaagctgaatgatctgtgcttcaccaacgtatacgccgactcgtttgtcatccgcggcgatgaagtgaggcagatcgcgcccggccagacgggcaagatcgccgactacaactacaagctgcccgatgatttcactggctgtgttattgcttggaatagtaacaatctggactcgaaggtcggggggaactacaactatctctataggctcttcagaaaaagtaatctgaagcctttcgagcgcgacatctcgactgagatatatcaggcaggctccaccccctgcaacggcgtggaggggtttaattgctacttcccccttcagagctacggctttcagccaacaaacggcgtaggctatcagccctaccgcgtggtggtgctgtcattcgaactcctgcacgcccccgcaaccgtctgcggccctaagaagagtacgaacctggtgaagaacaagtgcgtcaacttcaatttcaacggcctcaccggcacaggggtgctgaccgagtccaacaagaagtttctcccgttccagcagtttgggagggacatcgcagacactaccgacgcggtgagggacccacagaccttggagatactggacatcactccttgcagcttcgggggcgtgtcggtcataactcccggcactaacacctcaaaccaggtcgccgtgctctaccaggacgtgaactgtaccgaggtgcccgtggcgattcacgccgaccagctgacacccacgtggagggtgtactccaccggaagtaatgtcttccagacccgcgccgggtgtctgataggcgccgagcacgtcaacaactcgtacgagggatcctga

SEQ ID NO: 2 - protein, 663 aa, synthetic construct
MFVFLVLLPLVSSQCVNLTTRTQLPPAYTNSITRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYEGS

SEQ ID NO: 3 - protein, 240 aa, synthetic construct
QAVVTQEPSVTVSPGGTVILTCGSGTGAVTSGHYANWFQQKPGQAPRALIFDTDKKYSWTPGRFSGSLLGAKAALTISDAQPEDEAEYYCSLSDVDGYLFGGGTQLTVLSGGGGSGGGGSGGGGSGGGGSQAVVTQEPSVTVSPGGTVILTCGSGTGAVTSGHYANWFQQKPGQAPRALIFDTDKKYSWTPGRFSGSLLGAKAALTISDAQPEDEAEYYCSLSDVDGYLFGGGTQLTVLS

SEQ ID NO: 4 - protein, 240 aa, synthetic construct
QAVVTQEPSVTVSPGGTVILTCGSSTGAVTSGHYANWFQQKPGQAPRALIFETDKKYSWTPGRFSGSLLGAKAALTISDAQPEDEAEYYCSLSDVDGYLFGGGTQLTVLSGGGGSGGGGSGGGGSGGGGSQAVVTQEPSVTVSPGGTVILTCGSSTGAVTSGHYANWFQQKPGQAPRALIFETDKKYSWTPGRFSGSLLGAKAALTISDAQPEDEAEYYCSLSDVDGYLFGGGTQLTVLS

SEQ ID NO: 5 - protein, 280 aa, synthetic construct
QVQLQESGPGLVKPSETLSLTCTVSGASISSSHYYWGWIRQPPGKGPEWIGSMYYSGRTYYNPALKSRVTISSDKSKNQFFLKLTSVTAADTAVYYAAREGPTHYYDNSGPIPSDEYFQHWGQGTLVTVSGGGGSGGGGSGGGGSGGGGSQVQLQESGPGLVKPSETLSLTCTVSGASISSSHYYWGWIRQPPGKGPEWIGSMYYSGRTYYNPALKSRVTISSDKSKNQFFLKLTSVTAADTAVYYAAREGPTHYYDNSGPIPSDEYFQHWGQGTLVTVS

SEQ ID NO: 6 - protein, 252 aa, synthetic construct
QVQLVESEAEVKKPGSSVKVSCKASGGTFSSYAISWVRQAPGQGLEWMGGIIPIFGTANYAQKFQGRVTITADESTSTAYMELSSLRSEDTAVYYCVLLDTTMVTGYYFDYWGQGTLVTVSSGILGSGGGGSGGGGSGGGGSNFMLTQPPSASGTPGQSVTISCSGSGSNIGNNKVNWYQQLPGTAPKLLIYSNNQRPSGVPDRFSGSKSGTSASLAISGLQSEDEADYYCAAWDDSLNGYVFGTGTKLTVL

SEQ ID NO: 7 - protein, 250 aa, synthetic construct
QVQLQQGGAGLLKPSETLSLTCGVYGGSFSGYYWSWIRQSPGKGLEWIGEINHSGSANYNPSVKSRVTISVDTSKNQFSLQLSSVTAADTAVYYCARDRAVLTGEGWYFDLWGRGTLVTVSSGILGSGGGGSGGGGSGGGGSSYELTQPPSVSVSPGQTASITCSGDKLGDKYTCWYQQKPGQSPVLVLYEDTKRPSGIPERFSGSNSGNTATLTISRVEAGDEADYYCQLWDSSSDHYVFGSGTKLTVL

SEQ ID NO: 8 - protein, 252 aa, synthetic construct
QVQLVESEAEVKKPGSSVKVSCKASGGTFSSYAISWVRQAPGQGLEWMGGTIPIFGTADYAQEFQGRVTITTDESTSTAYMELSGLRSEDTAVYYCVLLGTTMVTGHYFDYWGQGTLVTVSSGILGSGGGGSGGGGSGGGGSNFMLTQPPSASGTPGQSVTISCSGSGSNIGNNKVNWYQQLPGTAPKLLIYSNNQRPSGVPDRFSGSKSGTSASLAISGLQSEDEADYYCAAWDDGLSGYVFGTGTKLTVL

SEQ ID NO: 9 - protein, 252 aa, synthetic construct
QVQLVESEAEVKKPGSSVKVSCKASGGTFSSYAISWVRQAPGQGLEWMGGTIPIFGTANYAQKFQGRVTITADESTSTAYMELSSLRSEDTAVYYCVLLGTTMVTGYYFDYWGQGTLVTVSSGILGSGGGGSGGGGSGGGGSNFTLTQPPSASGTPGQSVTISCSGSGSNIGNNKVNWYQQLPGTAPKLLIYSNNQRPSGVPDRFSGSKSGTSASLAISGLQSEDEADYYCAAWDDSLNGYVFGTGTKLTVL

SEQ ID NO: 10 - protein, 264 aa, synthetic construct
QVQLVESEGGLVQPGGSLRLSCAASGFTFSSYEMNWVRQAPGKGLEWVSRIDGDGSSTNYADSVKGRFTISRDNAKSTLYLQMNSLRAEDTAVYYCTRARYFGSVSPYGMDVWGQGTTVTVSSGILGSGGGGSGGGGSGGGGSDIRVTQSPSSVSASVGDRVTISCRASQGIATWLGWYQQKPGKPPQLLIYSASTLQTGVPSRFSGSGSGTDFTLTISSLQPEDVATYYCQEGSTFPLTFGGGTKVDIKSGILEQKLISEEDL

SEQ ID NO: 11 - protein, 163 aa, synthetic construct
SASTGSFDSWGQGTLVTVSSGILGSGGGGSGGGGSGGGGSQAVVTQEPSVTVSPGGTVILTCGSSTGAVTSGHYANWFQQKPGQAPRALIFETDKKYSWTPGRFSGSLLGAKAALTISDAQPEDEAEYYCLLSDVDGYLFGGGTQLTVLSGILEQKLISEEDL

SEQ ID NO: 12 - protein, 154 aa, synthetic construct
QVQLQESGPGLVKPSETLSLTCTVSGASISSSHYYWGWIRQPPGKGPEWIGSMYYSGRTYYNPALKSRVTISPDKSKNQFFLKLTSVTAADTAVYYCAREGPTHYYDNSGPIPSDEYFQHWGQGTLVTVSSGILGSGGGGSGGGGSGGGGLQEF

SEQ ID NO: 13 - protein, 268 aa, synthetic construct
QVQLQQWDAGLVKPSQTLSLTCAISGDSVSSNSAAWNWIRQSPSRGLEWPGRTYYRSKWQNNYALSVQGRITINPDTSNNQFSLQLDSMTPEDTGVYYCTRGGGSLDYWGQGTLVTVSSGSASAPTGILGSGGGGSGGGGSGGGGSSYELTQPPSVSVSPGQTATITCSGDEMGDKYAYWYQQKPGQAPVLVIYKDSERPSGIPERFSGSSSGTTVTLTISGVQAEDEADYYCQSADSSGTSVVFGGGTKVTVLSGILEQKLISEEDL

SEQ ID NO: 14 - protein, 124 aa, synthetic construct
QVQLQQSGPGLVRPSQTLSLTCAISGDSVPKNGASWNWIRLSPSRGLEWLGRTHYSSRWYHDYAFFVKSRITINVDTSETQVSLQLDSVTPDDTGVYYCARESQRRGWFDLWGQGTLVTVSQEF

SEQ ID NO: 15 - protein, 279 aa, synthetic construct
QVQLQQSGPGRVKPSQTLSLTCDISGDSVSSNSVAWNWIRQSPSRGLEWLGRTYYRSKWINEYGPFVRSRITINPDTSKNQFSLQLNSVTPEDTAVYYCATMANSGYDRSSGHNYGMDVWGQGTTVTVSSGSASAPTGILGSGGGGSGGGGSGGGGSSYELTQPPSVSVSPGQTARITCSGDALPKQYTYWYQQKAGQAPVLVIYKDTERPSGIPERFSGTSSGTTVTLTISGVQAEDEADYYCQSADSSGSYVFFGGGTKVTVLSGILEQKLISEEDL

SEQ ID NO: 16 - protein, 44 aa, synthetic construct
MKVLIVLLAIFAALPLALAQPVISTTVGSAAEGSLDKREAEAEY

SEQ ID NO: 17 - protein, 94 aa, synthetic construct
MRQVWFSWIVGLFLCFFNVSSAAPVNTTTEDETAQIPAEAVIGYLDLEGDFDVAVLPFSNSTNNGLLFINTTIASIAAKEEGVSLDKREAEAEY

SEQ ID NO: 18 - protein, 46 aa, synthetic construct
MESVSSLFNIFSTIMVNYKSLALALLSVSSLKYARGLDKREAEAEY

SEQ ID NO: 19 - protein, 99 aa, synthetic construct
MQLLRCFSIFSVIASVLAQELTTICEQIPSPTLESTPYSLSTTTILANGKAMQGVFEYYKSVTFVSNCGSHPSTTSKGSPINTQYVFGGGGSGGGGSEY

SEQ ID NO: 20 - protein, 99 aa, synthetic construct
MQLLRCFSIFSVIASVLAQELTTISEQIPSPTLESTPYSLSTTTILANGKAMQGVFEYYKSVTFVSNSGSHPSTTSKGSPINTQYVFGGGGSGGGGSEY

SEQ ID NO: 21 - DNA, 15 bp, unidentified
  source 1..15: mol_type = other DNA
agtcggtctcaagtg

SEQ ID NO: 22 - DNA, 13 bp, synthetic construct
  source 1..13: mol_type = other DNA
gtccactctggtc

SEQ ID NO: 23 - protein, 98 aa, synthetic construct
QVQLVESGGGLVQPGGSLRLSCAASGFTFDSYAMGWFRQAPGKEREXVSCISSSGGSTYYADSVKGRFTISRDNAKNTVYLQMNSLKPEGTAVYYCAA

SEQ ID NO: 24 - protein, 96 aa, Vicugna pacos
QVQLVESVGGLVQDGGSLRLSCAASGRTFSRSAMRWFRQAPGKEREWVSCISSSDGSTNYADSVKGRFTISRDNAKNTLYLQMNSLKPEGTAVYYC

SEQ ID NO: 25 - protein, 97 aa, Lama glama
QVQLVESVGGLVQDGGSLRLSCAASGRTFSRSAMRWFRQAPGKEREWVSCISSSDGSTNYADSVKARFTISRDNAKNTLYLQMNSLKPEDTAVYYCA

SEQ ID NO: 26 - protein, 97 aa, Lama glama
QVQLVESGGGLVQAGGSLRLSCAASGDTICISAMGWYRQAPGKERELVAAITSGGSTNYADSVKGRFTISRDNAKNTVYLQMNSLKPEDTAVYYCNA

SEQ ID NO: 27 - protein, 97 aa, Vicugna pacos
QVQLVESGGGLVQPGGSLRLSCAASGSIFSINAMGWYRQAPGKQRELVAAITSGGSTNYADSVKGRFTISRDNAKNTVYLQMNSLKPEDTAVYYCNA

SEQ ID NO: 28 - protein, 96 aa, Vicugna pacos
QVQLVESGGGLVQPGGSLRLSCAASGSIFSINAMGWYRQAPGKQRELVAAINTGGGSTYYADSVKGRFTISRDNAKNTLYLQMNSLKSEGTAVYYC

SEQ ID NO: 29 - protein, 96 aa, Vicugna pacos
QVQLVESGGGLVQPGGSLRLSCAASGFTLDNYAMGWFRQAPGKEREFVSCISWSGGSTYYADSVKGRFTISRDNAKNTVYLQMNSLKPEGTAVYYC

SEQ ID NO: 30 - protein, 96 aa, Vicugna pacos
EVQVVESGGGLVQPGGSLRLSCAASGFTLDYYAISWFRQAPGKEREGVSCINSSDGSTYYADSVKGRFTISRDNAKNTVYLQMNSLKPEGTAVYYC

SEQ ID NO: 31 - protein, 96 aa, Vicugna pacos
EVQLVESGGGLVQAGGSLRLSCAASGFTFDDYAIGWFRQAPGKEREGVSCISSSDGSTYYADSVKGRFTISSDNAKNTVYLQMNSLKPEGTAVYYC

SEQ ID NO: 32 - protein, 96 aa, Vicugna pacos
QLQLVESGGGLVQPGGSLRLSCAASGFTLDYYAIGWFRQAPGKEREGVSCISSSGGSTNYADSVKGRFTISRDNAKNTVYLQMNSLKPEDTAVYYC

SEQ ID NO: 33 - protein, 98 aa, Lama glama
QVQLVESGGGLVQPGGSLRLSCAASGFTLDDYAIGWFRQAPGKEREGVSCISSSDGSTYYADSVKGRFTISRDNAKNTLYLQMNSLKPEDTAVYYCAA

SEQ ID NO: 34 - protein, 96 aa, Vicugna pacos
QVQLVESGGGLVQAGGSLRLSCAASGFTLDYYAIGWFRQAPGKEREGVSCISSSDGSTYYADSVKGRFTISRDNAKNTVYLQMNSLKPEGTAVYYC

SEQ ID NO: 35 - protein, 96 aa, Vicugna pacos
QVQLVESGGGLVQPGGSLRLSCAASGFTLDYYAIGWFRQAPGKEREGVSCISSSDGSTYYADSVKGRFTISRDNAKNTVYLQMNSLKPEGPAVYYC

SEQ ID NO: 36 - protein, 98 aa, Lama glama
QVQLVESGGGLVQPGGSLRLSCAASGFTLDYYAIGWFRQAPGKEREGVSCISSSDGSTYYADSVKGRFTISRDNAKNTVYLQMNSLKPEDTAVYYCAA

SEQ ID NO: 37 - protein, 96 aa, Vicugna pacos
QVQLVESGGGLVQPGGSLRLSCAASGFTLDYYAIGWFRQAPGKEREGVSCISSSDGSTYYADSVKGRFTISRDNAKNTVYLQMNSLKPEGTAVYYC

SEQ ID NO: 38 - protein, 95 aa, Vicugna pacos
EVQLVESGGGLVQPGGSLRLSCAASGFTFSSYAMSWVRQAPGKERELVAAISSGGSTYYADSVKGRFTISRDNAKNTLYLQMNSLKSEGTAEYYC

SEQ ID NO: 39 - protein, 96 aa, Vicugna pacos
EVQVVESGGGLVQAGGSLRLSCAASGFTFSSYAMGWFRQAPGKEREFVAAISWSGGSTYYADSVKGRFTISRDNAKNTLYLQMNSLKPEGTAVYYC

SEQ ID NO: 40 - protein, 96 aa, Vicugna pacos
QVQLVESGGGLVQAGGSLRLSCAASGRTFSSYAMGWFRQAPGKEREFVAAISWIGGSTYYADSVKGRFTISRDNAKNTLYLQMNSLKPEGTAVYYC

SEQ ID NO: 41 - protein, 95 aa, Vicugna pacos
QVQLVESGGGLVQAGGSLRLSCAASGRTFSSYAMGWYRQAPGKERELVAAISSGGSTYYADSVKGRFTISRDNAKNTLYLQMNSLKPEGTAVYYC

SEQ ID NO: 42 - protein, 97 aa, Lama glama
QVQLVESGGGLVQAGGSLRHSCAASGLTFGSYAMGWYRQAPGKERELVAAISSGGSTYYADSVKGQFTISRDNAKNTLYLQMNSLKPGDTAVYYCAK

SEQ ID NO: 43 - protein, 95 aa, Vicugna pacos
QVQLVESGGGLVQAGGSLRHSCAASGLTFGSYAMGWYRQAPGKERELVAAISSGGSTYYADSVKGRFTISRDNAKNTLYLQMNSLKPEGTAVYYC

SEQ ID NO: 44 - protein, 95 aa, Vicugna pacos
QVQLVESGGGLVQAGGSLRHSCAASGLTFGSYAMGWYRQAPGKERELVAAISSGGSTYYADSVKGRFTISRDNAKNTVYLQMNSLKPEGTAVSYC

SEQ ID NO: 45 - protein, 136 aa, synthetic construct
  VARIANT 47..59: note = X can be any amino acid
  VARIANT 26..37: note = X can be any amino acid
  VARIANT 98..123: note = X can be any amino acid
QVQLVESGGGLVQAGGSLRLSCAASXXXXXXXXXXXXRQAPGKEREXXXXXXXXXXXXXYADSVKGRFTISRDNAKNTVYLQMNSLKPEDTAVYYCAXXXXXXXXXXXXXXXXXXXXXXXXXXYDYWGQGTQVTVS

SEQ ID NO: 46 - protein, 116 aa, synthetic construct
  VARIANT 26..37: note = X can be any amino acid
  VARIANT 47..59: note = X can be any amino acid
QVQLVESGGGLVQAGGSLRLSCAASXXXXXXXXXXXXRQAPGKEREXXXXXXXXXXXXXYADSVKGRFTISRDNAKNTVYLQMNSLKPEDTAVYYCAXXXXXXYDYWGQGTQVTVS

SEQ ID NO: 47 - protein, 135 aa, synthetic construct
  VARIANT 26..37: note = X can be any amino acid
  VARIANT 47..58: note = X can be any amino acid
  VARIANT 97..122: note = X can be any amino acid
QVQLVESGGGLVQAGGSLRLSCAASXXXXXXXXXXXXRQAPGKEREXXXXXXXXXXXXYADSVKGRFTISRDNAKNTVYLQMNSLKPEDTAVYYCAXXXXXXXXXXXXXXXXXXXXXXXXXXYDYWGQGTQVTVS

SEQ ID NO: 48 - protein, 115 aa, synthetic construct
  VARIANT 26..37: note = X can be any amino acid
  VARIANT 47..58: note = X can be any amino acid
  VARIANT 97..102: note = X can be any amino acid
QVQLVESGGGLVQAGGSLRLSCAASXXXXXXXXXXXXRQAPGKEREXXXXXXXXXXXXYADSVKGRFTISRDNAKNTVYLQMNSLKPEDTAVYYCAXXXXXXYDYWGQGTQVTVS

(19) United States
(12) Patent Application Publication
(10) Pub. No.: US 2025/0052765 A1
(43) Pub. Date: Feb. 13, 2025

(54) Nanobody Discovery Platform and Related Methods

(71) Applicant: Carnegie Mellon University, Pittsburgh, PA (US)

(72) Inventors: Christopher S. Szent-Gyorgyi, Pittsburgh, PA (US); Robert Van De Weerd, Pittsburgh, PA (US); Brigitte F. Schmidt, Pittsburgh, PA (US); Marcel P. Bruchez, Pittsburgh, PA (US)

(21) Appl. No.: 18/718,631

(22) PCT Filed: Dec. 16, 2022
(86) PCT No.: PCT/IB2022/062388

§ 371 Date: Jun. 11, 2024

Related U.S. Application Data
(60) Provisional application No. 63/290,408, filed on Dec. 16, 2021.

(51) Int. Cl. G01N 33/68; C07K 16/44

(52) U.S. Cl. CPC G01N 33/6854; C07K 16/44; C07K 2317/622; C07K 2319/60

(57) ABSTRACT

Provided herein are methods, nucleic acids, transformed yeast cells, and kits useful for rapid and effective multiplexed identification of nanobody binding to a target protein domain.

### CROSS REFERENCE TO RELATED APPLICATIONS

This application claims priority to U.S. Provisional Patent Application No. 63/290,408 filed Dec. 16, 2021, which is incorporated herein by reference in its entirety.

### STATEMENT REGARDING FEDERAL FUNDING

This invention was made with U.S. Government support under Grant No. GM126657 awarded by the National Institutes of Health. The U.S. Government has certain rights in this invention.

### SEQUENCE LISTING

The Sequence Listing associated with this application is filed in electronic format via EFS-Web and is hereby incorporated by reference into the specification in its entirety. The name of the XML file containing the Sequence Listing is 2207618.xml. The size of the XML file is 54,937 bytes, and the XML filed was created on Dec. 15, 2022.

Monoclonal antibodies have been mainstay reagents for biological and biomedical applications since Kohler and Milstein developed hybridoma fusions in the 1970s. Cloned IgGs, derived from immunization of mammals with purified proteins or protein complexes of interest, are used to specifically bind to and identify target proteins using technologies such as fluorescence microscopy, ELISA assay, and flow cytometry.

Despite the predominance of such antibody based technologies, the means by which monoclonal antibodies are generated and disseminated, together with the size and structure of IgGs, impose fundamental limitations on their usefulness, especially for research purposes. Immune tolerance largely prevents generation of monoclonal antibodies against many conserved mammalian protein epitopes. Poor specificity and documentation of commercial antibodies has led to a crisis in experimental reproducibility, with ˜50% of all antibodies being faulty, leading to an annual waste of ˜$350 million in US research expenditures. The highly diverse and specific protein repertoire targeted by research antibodies drives up costs of commercial monoclonals, if the desired antibodies are available. Rectifying this by the systematic generation of sequenced, validated monoclonals would cost an estimated $50,000 per antibody and would fail for many targets.

IgGs (4 chains totaling 150 kD) are refractory to fusion to other proteins and recombinant expression and purification except at great expense in mammalian cells (e.g. for therapeutic antibodies). The configuration of the IgG binding interface-dual instances of paired heavy (VH) and light (VL) chain variable domains-impedes binding to concave, inaccessible or small protein surfaces such as enzyme active sites, junctions between complexed proteins, and small inserted motifs; binding artifacts may be introduced by IgG bivalency. The size and sequence complexity of IgGs can make covalent labeling cumbersome and non-stoichiometric, and potentially interfere with binding specificity; bulky fluorophore-labeled IgGs diffuse excruciatingly slowly into and out of optically clarified brain and other prepared tissues.

To address these limitations, scientists have developed ˜50 alternative small single domain binding scaffolds over the past 20 years; Ig-derivative scaffolds are V_{H}, V_{L}, V_{HH}, and V-NAR, and important non-Ig scaffolds include: monobodies, anticalins, affibodies, and DARPins. V_{HH} domains of camelids (llamas, alpacas, dromedaries and camels) are closely related to mammalian Vu domains but lack associated V_{L} domains, yet bind protein targets with affinities comparable to IgGs. V_{HH} have typically been isolated by screening phage libraries derived from cDNA from an immunized camelid, from cDNA from an unimmunized camelid (a ‘naïve’ library, or from synthetically mutagenized VHH scaffolds. Their structure, including location of variable and complementarity determining regions (CDRs), are broadly-known (See, e.g., Mitchell, LS, Colwell, LJ. Comparative analysis of nanobody sequence and structure data. Proteins. 2018; 86:697-706. doi.org/10.1002/prot.25497 and Mitchell L S, Colwell L J. Analysis of nanobody paratopes reveals greater diversity than classical antibodies. Protein Eng Des Sel. 2018-7-1; 31 (7-8): 267-275). Several companies now offer services for isolating V_{HH} (aka nanobody, NB) by screening of immune or synthetic phage libraries.

Traditionally, V_{HH} peptides may be displayed on phage, bacteria or yeast cells, or on ribosomes. Isolation of individual clones encoding candidate binding scaffolds may be achieved by affinity panning or FACS; candidates may be then characterized for binding and individually sequenced. Promising binding scaffolds may be improved by directed evolution. Target protein is generally provided as labeled soluble protein for FACS of bacteria or yeast, or as unlabeled protein immobilized on a capture surface for panning of phage or ribosomes; limited multiplexing can be achieved using several exogenous proteins. Proteins expressed by a yeast cell can be secreted and captured on its surface using biotin/streptavidin chemistry, but the captured protein must still be probed by exogenous protein. Current nonimmune scaffold screens use purified target protein to isolate candidate binders that are physically cloned and individually evaluated.

More effective, and less expensive and resource-intensive NB screening methods and reagents are needed.

### SUMMARY

According to an aspect of the invention, a nucleic acid is provided comprising one or more inducible gene for expressing in a yeast cell: a first fusion protein comprising amino acid sequences of: an anchor peptide selected for anchoring the first fusion protein in a cell wall of the cell, a nanobody, and an scFv (FAP) for binding a first fluorophore; and a second fusion protein comprising amino acid sequences of a secretion signal peptide and an scFv (FAP) for binding a second fluorophore, wherein the first fluorophore increases fluorescent emissions, e.g., at least 1000-fold, at an excitation wavelength when bound by the scFv of the first fusion protein, the second fluorophore increases fluorescent emissions, e.g., at least 1000-fold, at an excitation wavelength when bound by the scFv of the second fusion protein, and the fluorescent emission of the scFv-bound first fluorophore is detectably different from that of the scFv-bound second fluorophore, wherein the nucleic acid further comprises a barcode for identifying the TPD in the coding sequence of the first fusion protein. The nucleic acid may be provided in a yeast cell. Also provided is a plurality of the nucleic acids, with at least 10⁷, at least 10⁸, or at least 10⁹ different V_{HH} CDR sequences. The plurality of nucleic acids may be provided as a library of transformed yeast cells comprising, e.g., transformed with, the plurality of nucleic acids.

According to another aspect of the invention, a kit is provided, comprising the nucleic acid or nucleic acids as described in the preceding paragraph, optionally provided in one or more yeast cells, and a tethered dye comprising the first fluorophore tethered by a spacer of between 1 nm and 50 nm to the second fluorophore, wherein the first FAP binds the first fluorophore of the tethered dye and the second FAP binds the second fluorophore of the tethered dye, the spacer optionally comprising one or more PEG₁₋₁₀₀ groups. A kit is also provided comprising two or more part plasmids comprising components for assembly of a nucleic acid as described above using a Golden Gate cloning method, comprising: a first nucleic acid plasmid, comprising two different Type IIS restriction endonuclease recognition sites; and one or more additional plasmids comprising sequences flanked by recognition sites of a Type IIS restriction endonuclease, and encoding: one or more secretion leader peptides; an E. coli replication origin and antibiotic resistance gene, such as AmpR; a Gal1-10 UAS bicistronic promoter; a first fluorogen activating protein; a second fluorogen activating protein, for activating a fluorogen different from the first fluorogen-activating protein; a V_{HH} peptide sequence; a SAG1 linker; a transcription terminator, such as an ADH1 terminator; a first selection marker, such as a TRP1 gene; a yeast replication origin; an optional control Vun peptide, such as Lag27; a second selection marker, such as a KanR gene; and an AGA2 anchor sequence and a linker peptide, each of which being flanked by recognition sites of one of the two different Type IIS restriction endonuclease recognition sites of the first plasmid.

According to a further aspect of the invention, a method of identifying a nanobody that binds a target protein domain is provided, comprising: growing a yeast cell as described above in yeast culture medium; inducing expression of the first fusion protein and the second fusion protein by the yeast cell; contacting the yeast cell with a tethered dye comprising cognate fluorogen moieties of the first FAP and the second FAP, wherein the cognate fluorogen moieties are separated by a PEG spacer of from 1 nm to 50; illuminating the yeast cell with light at an excitation wavelength of the cognate fluorogen moieties bound to their cognate FAPs; and determining if the yeast cell fluoresces at emission wavelengths of both FAP-bound fluorogen moieties, indicating binding of the first fusion peptide to the second fusion peptide.

The following clauses outline additional aspects, embodiments, and/or examples of the present invention.

Clause 1. A nucleic acid comprising one or more inducible gene for expressing in a yeast cell:
a first fusion protein comprising amino acid sequences of: an anchor peptide selected for anchoring the first fusion protein in a cell wall of the cell, a nanobody, and an scFv (FAP) for binding a first fluorophore; and
a second fusion protein comprising amino acid sequences of a secretion signal peptide and an scFv (FAP) for binding a second fluorophore,
   wherein the first fluorophore increases fluorescent emissions, e.g., at least 1000-fold, at an excitation wavelength when bound by the scFv of the first fusion protein, the second fluorophore increases fluorescent emissions, e.g., at least 1000-fold, at an excitation wavelength when bound by the scFv of the second fusion protein, and the fluorescent emission of the scFv-bound first fluorophore is detectably different from that of the scFv-bound second fluorophore, wherein the nucleic acid further comprises a barcode for identifying the TPD in the coding sequence of the first fusion protein.

Clause 2. The nucleic acid of clause 1, wherein the second peptide comprises a target protein domain.

Clause 3. The nucleic acid of clause 1 wherein the first fusion protein comprises amino acid sequences of: in order from an N-terminal to a C-terminal direction, the anchor peptide, the nanobody, and the scFv for binding a first fluorophore; and the second fusion protein comprises amino acid sequences of in order from an N-terminal to C-terminal direction, the secretion signal peptide and the scFv for binding a second fluorophore.

Clause 4. The nucleic acid of clause 1, wherein the second fusion protein comprises amino acid sequences of, in order from an N-terminal to C-terminal direction, the secretion signal peptide, a target protein domain, and the scFv for binding a second fluorophore.

Clause 5. The nucleic acid of any one of clauses 1-4, wherein the yeast cell is a Pichia pastoris, Saccharomyces cerevisiae, Kluyveromyces lactis, or Hansenula polymorpha cell.

Clause 6. The nucleic acid of any one of clauses 1-5, wherein the one or more genes encoding the first and second fusion proteins are bicistronic.

Clause 7. The nucleic acid of clause 6, wherein the genes are under control of a GAL1-10 (GAL1/GAL10) UAS bidirectional promoter.

Clause 8. The nucleic acid of any one of clauses 1-7, wherein one of the first and second fluorophore is a triaryl methine dye, such as malachite green.

Clause 9. The nucleic acid of any one of clauses 1-7, wherein one of the first and second fluorophore is a thiazole orange dye.

Clause 10. The nucleic acid of any one of clauses 1-7, wherein one of the first and second fluorophore is a malachite green dye and the other of the first and second fluorophore is a thiazole orange dye.

Clause 11. The nucleic acid of clause 10, wherein one of the first and second fluorophore is malachite green and the other of the first and second fluorophore is sulfonated thiazole orange.

Clause 12. The nucleic acid of clause any one of clauses 1-11, wherein the first fusion peptide comprises one or more type IIS restriction endonuclease recognition site-terminated DNA modules.

Clause 13. The nucleic acid of clause 12, comprising a plurality of type IIS restriction endonuclease recognition site-terminated DNA modules comprising sequences encoding in separate modules:
a first FAP open reading frame (ORF);
an optional TPD ORF;
a secretion leader ORF;
a bidirectional promoter;
a surface display leader ORF, optionally including a TPD barcode;
a NB ORF;
optionally a SAG1 linker ORF; and/or
a second FAP ORF.

Clause 14. The nucleic acid of clause 13, comprising a plurality of type IIS RE recognition site-terminated DNA modules comprising sequences in order and encoding: a first FAP ORF, a TPD ORF, a secretion leader ORF, a bidirectional promoter, a surface display leader ORF optionally including a TPD barcode, a NB ORF, an optional SAG1 linker ORF, and a second FAP ORF, wherein the bidirectional promoter directs transcription of a first fusion peptide comprising, in order, the secretion leader, the TPD, and the first FAP, and of a second fusion protein comprising, in order, the surface display leader, optionally the TPD barcode, the NB ORF, optionally the SAG1 linker, and the second FAP.

Clause 15. The nucleic acid of clause 13, further comprising one or more of the following type IIS RE recognition site-terminated DNA modules: a terminator sequence 3′ to the modules comprising the first and second FAP ORFs, such as an ScADH1 or CYC1 terminator sequence; one or more selection markers, such as TRP1 or KanR; a yeast replication origin; and/or an E. coli selection marker and replication origin.

Clause 16. The nucleic acid of any one of clauses 1-13, wherein the first FAP and the second FAP have scFv sequences of two of SEQ ID NOS: 3-15.

Clause 17. The nucleic acid of any one of clauses 1-16, wherein the camelid V_{HH} nanobody comprises the sequence of, or a sequence having at least 90%, at least 95%, or at least 99% sequence identity with the sequence:

(SEQ ID NOS: 23 and 45-48)
QVQLVESGGGLVQAGGSLRLSCAAS(X)ₘRQAPGKERE(X)ₙYADSVKGR
FTISRDNAKNTVYLQMNSLKPEDTAVYYCA(X),YDYWGQGTQVTVS,

excluding (X)ₘ, (X)ₘ, and (X)ₚ, wherein n is 12, m is 12 or 13, and p ranges from 6 to 26, inclusive, and (X)ₘ, (X)ₙ, and (X)ₚ correspond to CRD1, CDR2, and CDR3, respectively of the camelid V_{HH}.

Clause 18. The nucleic acid of any one of clauses 1-17, comprising a barcode sequence in the sequence encoding the second fusion protein identifying the TPD of the first fusion protein.

Clause 19. The nucleic acid of clause 1, wherein the second fusion protein does not comprise a sequence encoding a TPD.

Clause 20. The nucleic acid of any one of clauses 1-19, wherein the TPD is an ectodomain or a fragment thereof of: a viral surface protein, a cancer-associated cell surface marker; a neural protein; a bacteria surface protein, or a parasite surface protein.

Clause 21. A plurality of nucleic acids as described in any one of clauses 1-20, with at least 10⁷, at least 10⁸, or at least 10⁹ different V_{HH} CDR sequences.

Clause 22. A yeast cell comprising the nucleic acid of any one of clauses 1-20.

Clause 23. The yeast cell of clause 22, comprising an S. cerevisiae EBY100 cell transformed with the nucleic acid.

Clause 24. A library of yeast cells comprising different colony forming units, with each colony forming unit corresponding to individual nucleic acids of the plurality of nucleic acids of clause 21.

Clause 25. The library of yeast cells of clause 24, comprising S. cerevisiae EBY100 cells transformed with the plurality of nucleic acids.

Clause 26. A kit comprising the nucleic acid or nucleic acids of any one of clauses 1-21, optionally provided in one or more yeast cells, and a tethered dye comprising the first fluorophore tethered by a spacer of between 1 nm and 50 nm to the second fluorophore, wherein the first FAP binds the first fluorophore of the tethered dye and the second FAP binds the second fluorophore of the tethered dye, the spacer optionally comprising one or more PEG1 100 groups.

Clause 27. A kit comprising two or more part plasmids comprising components for assembly of a nucleic acid of any one of clauses 1-20 using a Golden Gate cloning method, comprising:
a first nucleic acid plasmid, comprising two different Type IIS restriction endonuclease recognition sites; and
one or more additional plasmids comprising sequences flanked by recognition sites of a Type IIS restriction endonuclease, and encoding:
   one or more secretion leader peptides;
   an E. coli replication origin and antibiotic resistance gene, such as AmpR;
   a Gal1-10 UAS bicistronic promoter;
   a first fluorogen activating protein;
   a second fluorogen activating protein, for activating a fluorogen different from the
   first fluorogen-activating protein;
   a VHH peptide sequence;
   a SAG1 linker;
   a transcription terminator, such as an ADH1 terminator;
   a first selection marker, such as a TRP1 gene;
   a yeast replication origin;
   an optional control VHH peptide, such as Lag27;
   a second selection marker, such as a KanR gene; and
   an AGA2 anchor sequence and a linker peptide,
   each of which being flanked by recognition sites of one of the two different Type IIS
   restriction endonuclease recognition sites of the first plasmid.

Clause 28. The kit of clause 27, wherein the kit comprises five additional plasmids, including:
a second plasmid comprising a sequence encoding the AGA2 anchor sequence and a linker peptide and comprising a barcode nucleic acid sequence;
a third plasmid comprising the sequence encoding the a Gal1-10 UAS bicistronic promoter;
a fourth plasmid comprising a plurality of sequences encoding two or more secretion leaders;
a fifth plasmid comprising a sequence encoding the E. coli replication origin and antibiotic resistance gene, such as AmpR; and
a sixth plasmid comprising sequences encoding a first fluorogen activating protein; a second fluorogen activating protein, for activating a fluorogen different from the first fluorogen-activating protein; a V_{HH} peptide sequence; a SAG1 linker; a transcription terminator, such as an ADH1 terminator; a first selection marker, such as a TRP1 gene; a yeast replication origin; and a second selection marker, such as a KanR gene.

Clause 29. The kit of clause 28, further comprising from 10 to 1,000 different versions of the second plasmid with from 10 to 1,000 different barcode sequences.

Clause 30. The kit of clause 28 or 29, further comprising at least 10⁹ different versions of the sixth plasmid comprising V_{HH}-encoding sequences encoding at least 10⁹ different V_{HH} peptides.

Clause 31. The kit of any one of clauses 27-30, further comprising S. cerevisiae cells, such as EBY100 cells, and optionally E. coli cells for transformation and propagation of the plasmids.

Clause 32. The kit of any one of clauses 26-31, wherein the two different Type IIS restriction endonuclease recognition sites of the first plasmid are BsaI and BsmB1 recognition sites, and the one of the two different Type IIS restriction endonuclease recognition sites of the one or more additional plasmids are BsaI recognition sites.

Clause 33. The kit of any one of clauses 26-32, further comprising two different Type IIS restriction endonucleases, corresponding to the two different Type IIS restriction endonuclease recognition sites of the first plasmid.

Clause 34. The kit of any one of clauses 26-33, wherein the plasmids are provided in transformed E. coli cells.

Clause 35. The kit of any one of clauses 26-34, further comprising a tethered dye comprising the first fluorophore tethered by a spacer of between 1 nm and 50 nm to the second fluorophore, wherein the first FAP binds the first fluorophore of the tethered dye and the second FAP binds the second fluorophore of the tethered dye, the spacer optionally comprising one or more PEG1 100 groups.

Clause 36. The kit of any one of clauses 26-35, further comprising fluorogens MG-2p and TO1-2p.

Clause 37. The kit of any one of clauses 26-36, wherein the first FAP and the second FAP have scFv sequences of two of SEQ ID NOS: 3-15, and one of the first and second fluorophore are malachite green and thiazole orange, and the other of the first and second fluorophore are the other of malachite green and thiazole orange.

Clause 38. The kit of clause 26 or 35, wherein the spacer is cleavable.

Clause 39. The kit of clause 38, wherein the spacer comprises a disulfide bond or a nitro-benzyl moiety.

Clause 40. The kit of clause 26 or 35, wherein the tethered dye has a structure chosen from:

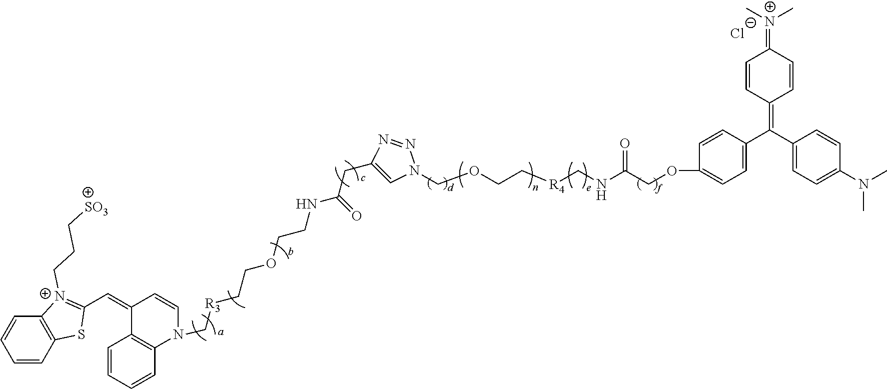

wherein a is an integer from 1 to 6, e.g., from 1 to 4, from 2 to 4, or 3; b is an integer from 1 to 200, e.g., from 1 to 100, from 2 to 50, or 2; c is an integer from 1 to 6, e.g., from 1 to 4, from 1 to 3, or 2; d is an integer from 1 to 6, e.g., from 1 to 4, from 1 to 3, or 2; n is an integer from 1 to 200, e.g., from 1 to 100, from 2 to 100, from 75 to 85, or 80; e is an integer from 1 to 6, e.g., from 1 to 4, from 1 to 3, or 2; f is an integer from 1 to 6, e.g., from 1 to 4, from 2 to 4, or 3; and R3 and R4 each independently are

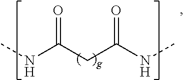

wherein g is an integer from 1 to 6, e.g., from 1 to 4, from 1 to 3, or 2;

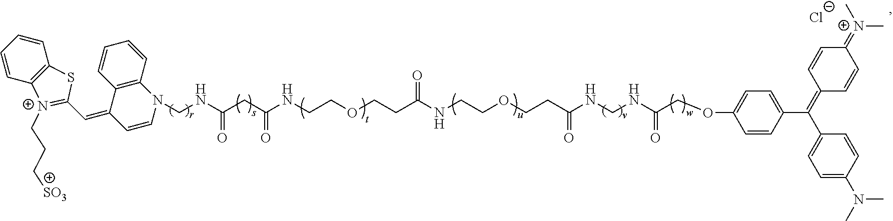

wherein r is an integer from 1 to 6, e.g., from 1 to 4, from 1 to 3, or 3; s is an integer from 1 to 6, e.g., from 1 to 4, from 1 to 3, or 3; t is an integer from 1 to 200, e.g., from 1 to 100, from 2 to 50, or 2; u is an integer from 1 to 200, e.g., from 1 to 100, from 2 to 100, from 75 to 85, or 80; v is an integer from 1 to 6, e.g., from 1 to 4, from 1 to 3, or 2; and w is an integer from 1 to 6, e.g., from 1 to 4, from 1 to 3, or 3; or

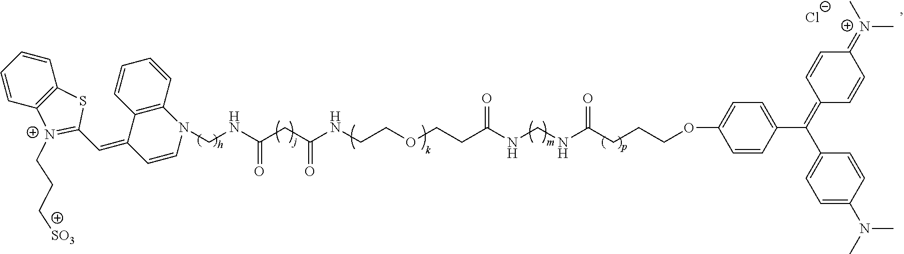

wherein h is an integer from 1 to 6, e.g., from 1 to 4, from 1 to 3, or 3; j is an integer from 1 to 6, e.g., from 1 to 4, from 1 to 3, or 2; k is an integer from 1 to 200, e.g., from 1 to 100, from 2 to 100, from 75 to 85, or 80; m is an integer from 1 to 6, e.g., from 1 to 4, from 1 to 3, or 2; and p is an integer from 1 to 6, e.g., from 1 to 4, from 1 to 3, or 3.

Clause 41. The kit of clause 40, wherein the tethered dye has a structure:

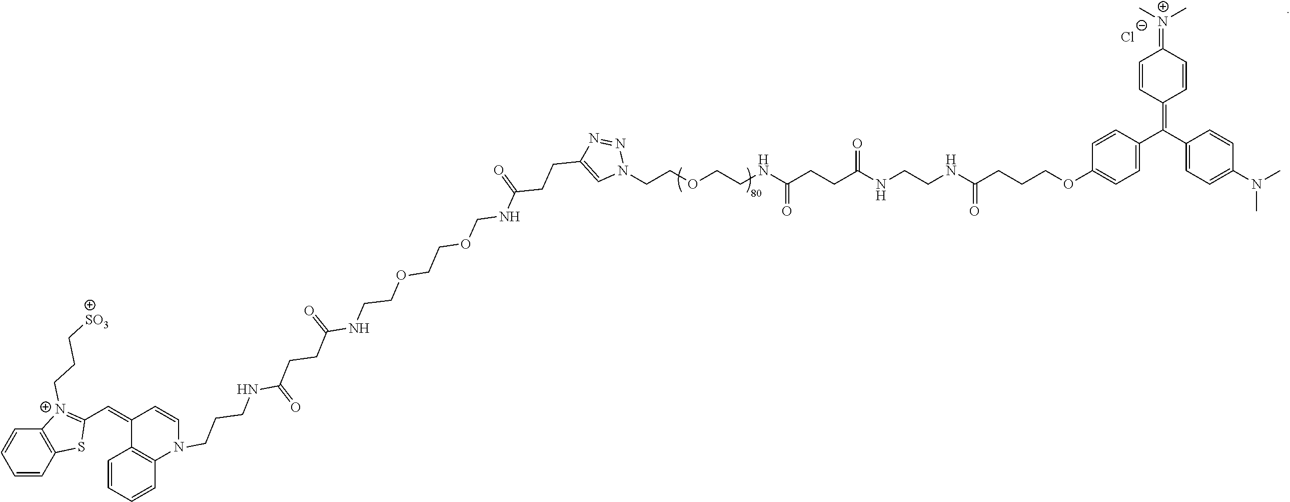

Clause 42. The kit of clause 26 or 35, wherein the tethered dye is attached to a magnetic bead.

Clause 43. A method of identifying a nanobody that binds a target protein domain, comprising:
growing a yeast cell according to any one of clauses 22-25 in yeast culture medium;
inducing expression of the first fusion protein and the second fusion protein by the yeast cell;
contacting the yeast cell with a tethered dye comprising cognate fluorogen moieties of the first FAP and the second FAP, wherein the cognate fluorogen moieties are separated by a PEG spacer of from 1 nm to 50;
illuminating the yeast cell with light at an excitation wavelength of the cognate fluorogen moieties bound to their cognate FAPs; and
determining if the yeast cell fluoresces at emission wavelengths of both FAP-bound fluorogen moieties, indicating binding of the first fusion peptide to the second fusion peptide.

Clause 44. The method of clause 43, wherein a library of yeast cells comprising at least 10⁷, at least 10⁸, or at least 10⁹ different nucleic acids having the same TPD, but different V_{HH} sequences, is grown and contacted with the tethered dye, and determining if the yeast cell fluoresces at emission wavelengths of both FAP-bound fluorogen moieties is performed by flow cytometry, and further comprising collecting yeast cells that fluoresce at emission wavelengths of both FAP-bound fluorogen moieties.

Clause 45. The method of clause 44, wherein the nucleic acid for expressing the fusion proteins, comprises a barcode sequence in the sequence encoding the second fusion protein identifying the TPD of the first fusion protein, and further comprising sequencing at least a portion of the sequence encoding the second fusion protein to obtain a sequence of at least the barcode and of one or more CDRs of the V_{HH}-encoding portion of the nucleic acid for expressing the fusion proteins.

Clause 46. The method of clause 45, wherein next generation sequencing is used to perform the sequencing.

Clause 47. The method of any one of clauses 43-46, wherein the nucleic acids encoding the first and second fusion proteins comprising a plurality of type IIS restriction endonuclease recognition site-terminated DNA modules comprising sequences encoding in separate modules:
the first FAP open reading frame (ORF);
the TPD ORF;
a secretion leader ORF;
a bidirectional promoter, such as GAL1/10;
a surface display leader ORF including a TPD barcode;
the V_{HH} ORF;
a SAG1 linker ORF; and
the second FAP ORF.

Clause 48. The method of clause 47, the nucleic acid comprising a plurality of type IIS RE recognition site-terminated DNA modules comprising sequences in order and encoding: the first FAP ORF, the TPD ORF, the secretion leader ORF, the bidirectional promoter, the surface display leader ORF including a TPD barcode, the NB ORF, the SAG1 linker ORF, and the second FAP ORF, wherein the bidirectional promoter directs transcription of a first fusion peptide comprising, in order, the secretion leader, the TPD, and the first FAP, and of a second fusion protein comprising, in order, the surface display leader, optionally the TPD barcode, the NB ORF, optionally the SAG1 linker, and the second FAP.

Clause 49. The method of clause 47, the nucleic acid further comprising one or more of the following type IIS RE recognition site-terminated DNA modules: a terminator sequence 3′ to the module comprising the first FAP ORF, such as an ScADH1 terminator sequence; one or more selection markers, such as TRP1 or KanR; a yeast replication origin; and/or an E. coli selection marker and replication origin.

Clause 50. The method of any one of clauses 43-49, wherein the first FAP and the second FAP have scFv sequences of two of SEQ ID NOS: 3-15.

Clause 51. The method of any one of clauses 43-50, wherein the VHH comprises the sequence of, or a sequence having at least 90%, at least 95%, or at least 99% sequence identity with the sequence:

(SEQ ID NOS: 23 and 45-48)
QVQLVESGGGLVQAGGSLRLSCAAS(X)ₘRQAPGKERE(X)ₙYADSVKGR
FTISRDNAKNTVYLQMNSLKPEDTAVYYCA(X),YDYWGQGTQVTVS,

excluding (X)ₘ, (X)ₙ, and (X)ₚ, wherein n is 12, m is 12 or 13, and p ranges from 6 to 26, inclusive, and (X)ₘ, (X)ₙ, and (X)ₚ correspond to CRD1, CDR2, and CDR3, respectively of the camelid V_{HH}.

Clause 52. The method of any one of clauses 43-51, further comprising, prior to growing the yeast cell, cloning a TPD into the nucleic acid.

Clause 53. The method of any one of clauses 43-52, wherein the TPD is an ectodomain or a fragment thereof of: a viral surface protein, a cancer-associated cell surface marker, a neural protein, a bacteria surface protein, or a parasite surface protein.

Clause 54. The method of any one of clauses 43-52, wherein the library of yeast cells represents two or more different TPDs.

### BRIEF DESCRIPTION OF THE DRAWINGS

FIG. 1 Schematic overview of V_{HH} and V_{H} domains. V_{HH}s (Nbs) are the variable domain derived from camelid HcAbs (A), whereas V_{H}s are the heavy chain variable domain derived from classical Abs (B). These homologous immunoglobulin domains have four key differences, represented in (C): (i) Nb H1 loops tend not to fit canonical conformations seen in Ab H1 loops, (ii) Nb H3 loops can be longer in length, (iii) Nb framework regions (FRs) are more conserved in both sequence and structure than Ab FRs and (iv) Nb FR2 have four solubility-enhancing mutations that facilitate stability without a partnering VL domain. Reproduced from Mitchell L S, et al. Protein Eng Des Sel. 2018-7-1; 31 (7-8): 267-275.

FIGS. 2A-2B: Nucleotide sequence for SARS-CoV-2-S1 (1992 base pairs, SEQ ID NO: 1) (FIG. 2A) and amino acid sequence for SARS-CoV-2-S1 (663 amino acids, SEQ ID NO: 2) (FIG. 2B). These sequences are provided as examples of sources of sequences for TPDs.

FIGS. 3A and 3B provide exemplary amino acid sequences of FAP proteins (SEQ ID NOS: 3-15).

FIG. 4 shows schematically an exemplary structure of the tie-dye or tethered dye described herein.

FIG. 5 provides a flow diagram of an overall process described herein.

FIG. 6. Combinatorial analysis using query sets: (A) An TPD query member samples an entire standard NB library, binding 4 NBs. (B) 3 different query sets sample the same standard NB library. (C) Query sets in (B) cumulatively analyzed in a single±binding matrix.

FIG. 7 is a diagram comparing a classical NB discovery platform with an example of the platform described herein.

FIG. 8. Stabilization and modulated dissociation of yeast displayed ternary complex in model system. (A) Well-characterized NBs and cognate TPDs (G: GFP; M: mCherry) comprise a model system. (B) gFAP is fused to Aga2p that is part of the Aga1p-Aga2p display scaffold (not shown). The ternary complex is stabilized by 3 bivalent interactions between the 3 components NB-gFAP, TPD-rFAP, and TD. Upon binding of the TD, red and green fluorescence is activated in a characteristic red/green ratio. After cleavage of TD, the TPD-rFAP is released at a rate determined by 1 monovalent interaction (NB/TPD). The TPD-rFAP/MG-PEG fragment is approximated as a covalent complex (K_{D}=10 pM) here and in (C); under conditions of high dilution of TPD-rFAP, the K_{off} step will dominate and lead to loss of cell-associated red fluorescence. An excess of monovalent green fluorogen is added to the system to maintain a constant green signal, as shown. The time dependent decrease in red/green ratio is the basis of the synchronized off-rate screen (SORS). (C) Model of ternary complex dissociation kinetics. Time courses of release of TPD-rFAP/TD from washed surface-bound 100 nM ternary complex were simulated in Berkeley-Madonna software at various gFAP/TD affinities.

FIG. 9. Yeast FAP display generates surface specific orthogonal fluorogenic signal. Mixed cell population of yeast displaying gFAP or a rFAP in the presence of 1 μM of each fluorogen. Nonfluorescent cells have lost their FAP expression plasmid.

FIG. 10. Yeast Secrete and Display (YSD) dual FAP reporter strains displaying gFAP/NB and secreting rFAP/TPD non- or cognate pairs. Yeast strains expressing rFAP/gFAP cognate pair GFP/L16, non-cognate pair thioredoxin Trx/L18 and d15/L18 without the TPD. Added fluorogens 1 μM.

FIG. 11 provides the structure of an exemplary TD.

FIG. 12. Fluorogens and cleavable Tie-dyes: “Linker” refers to PEG linker: (i) Chemical structures of the cleavable MG PEG tethered TO1 TDs. The disulfide S-S cleavable TD with TCEP and nitro-benzyl photo-cleavable TD. (ii) Chemical structures of high MW PEGylated fluorogens TO1 (PEG₁₋₂₀ₖ) and MG (PEG₁₋₂₀ₖ).

FIGS. 13A and 13B. FIG. 13A: YSD plasmids. Surface displayed NB-gFAP fusion protein (AM2-2 or looser variants) is outlined in green; secreted TPD-rFAP fusion protein is outlined in red. kex2: yeast leader cleavage site; P, promoter: T, terminator; KanR, TRP1, yeast selection markers; M, modules of the GG. The diagram of module (bottom) M6 supports linkage of a unique barcode to a TPD, and includes primer sites for paired-end NGS (next-generation sequencing). FIG. 13B: sequences of exemplary leaders for the fusion proteins described herein (SEQ ID NOS: 16-20).

FIG. 14. YSD vectors designed to end-user vector kits for the NB discovery platform. The 13 module YSD vectors are redesigned to construct an end user friendly 4 component GG assembly system compatible with the combinatorial NB discovery technology that is composed of 2 routes. Route (A) YSD TPD secretion test system; GG vector assembly to test and validate the TPD/leaders' for yeast secretion and (B) YSD NB library system; GG vector assembly to fuse the verified TPD/leader of (A) with the NB libraries to create the TPD-NB library vectors. The TPD-NB libraries will influx into the NB discovery platform.

FIG. 15 depicts schematically an exemplary GG cloning method for production of a YSD vector (NOS: 21 and 22).

FIGS. 16A and 16B. Synthetic nanobody library design. View of the overall NB architecture for the library design are shown with the NB framework sequences (Fr1-Fr4) and the CDRs (xCDR1/2 and CDR3) regions (FIG. 16A. SEQ ID NO: 23). The full framework germline consensus is shown (lower panel and FIG. 16B). The NB library 1 and 2 are shown with the number of different CDRs combinations to create the 10⁹ NB libraries (in red). FIG. 16B expands the image of the lower panel (SEQ ID NOS: 24-44).

FIG. 17 (Prior Art) provides an exemplary synthesis scheme for TD compound 5 (from, Ackerman D S. Vasilev K V. Schmidt B F. Cohen L B. Jarvik J W. Tethered Fluorogen Assay to Visualize Membrane Apposition in Living Cells. Bioconjug Chem. 2017-5-17; 28 (5): 1356-1362).

FIG. 18. Model NB discovery platform identifies cognate NB-TPD pairs in test YSD combinatorial screen. (A) Cartoon representation of the overall pair-end read NGS Library design as well and construction. NGS PCR1 amplifies the target amplicon (BC+NBs). PCR2 (full NGS library) adds the indexes I5 and I7 and flow-cell binding sequences for the MiSeq NGS platform. R1 (Fw) and R2 (Rv) reads are each 300 bps. (B) Tape Station gel analysis to confirm the purity of 5 NGS libraries. (C) L2 (Yeast) directly analyzed vectors using MiSeq platform data combined with custom-designed NGS software pipeline. This validates the NGS library design and the correct NGS readout. (D) FACS-DIVA cell-sorting experiment of three combinatorial YSD libraries with TD; 2-color sorted on high green signal (all displayed NBs) and either low to medium/high (L, M/H) levels of red signal (TPDs captured by NBs). Sorted YSD cells were regrown, NGS libraries constructed, and NGS implemented on the Miseq platform. (E) NGS results of the test combinatorial NB library screens of the 3 YSD libraries (+TD; specific capture of NB-TPDs cognate pairs). The test library screen proves we can identify the NB+barcode sequences of characterized NB/TPD partners (*NB/TPD). (F) Table of the NGS results of test library screen showing the pulls-out of expected cognate NBs whereas non-cognate “pairs” (light green) and the dl5 (no TPD, dark green) control are not. This validates our NB screen and strongly suggests that the model NB discovery platform works.

FIG. 19. Accuri flow cytometry experiments of YSD strains with replaced barcode. The replacement of the barcode does not influence NB display (left panel). Secreted GFP (red fluorescence) interacts with the cognate NBs and forms stable NB-GFP complexes (middle panel). GFP/L18 barcode adapted strains show no difference in TPD capture (right panel). Flow cytometry events 80K.

FIG. 20 provides graphs showing NB recognition & capture of secreted TPDs on the cell surface and their quantitation: model strains provide detailed information using a plate reader.

FIG. 21. Large PEGylated MG fluorogens are excluded from yeast periplasmic space. Back-ground red fluorescence of rFAP (dl5) of the periplasmic space is eliminated by using fluorogens MG-5 kPEG and higher; MG-B-Tau; control staining cell surface & the periplasmic space. L to H; low MW PEG to high.

FIG. 22. Release of TPDs from the periplasmic space. Shown is outer surface TPD-dL5 fluorescence as assayed by TD after incubation in assay buffer. All tested secreted NB/TPD ectodomain complexes display this behavior. Suc2-dL5 is a periplasmic invertase that is intensely fluorescent when analyzed by a small MW MG fluorogen (not shown), but not by TD. nc=non-cognate.

FIG. 23. TD efficiently captures NB/TPD complexes over a wide range of affinities. Shown are 2-D FACS dot plots of yeast NB/GFP strains with affinities as noted. Green numbers are NB-gFAP signal; red numbers are TPD-rFAP signals. In vivo, cells treated with 300 nM TD during 3-day cell culture; in vitro, cells treated with TD after harvest in assay buffer for 4 hours.

FIG. 24. (A) TD efficiently captures NB/TPD complexes. NB/TPD complexes are specifically captured for cognate NB/TPDs but not if the TPD is absent (dL5) or is not a cognate (nc mCh). (B) Bleaching TO1 moiety of TD leads to partial loss of TPD signal. Flow cytometer histograms of LaG18/GFP strain labeled during cell culture with 300 nM TD. Mean values are shown; unbleached (red), bleached for 90 sec at 525 nm (blue), and unlabeled control cells (orange).

FIG. 25. SORS sampling strategy: Ratiometric FACS model shows dissociation curves of cells with labeled TPD-rFAP off-rates (s⁻¹). Time samples of 4 minutes duration (I-V, blue) correspond to sorting of cells into bins A-D with indicated ranges of red/green ratios. Prior to TD cleavage at T=0, all cells will reside in bin I-A. The cells within each bin are outgrown, DNA is isolated, and barcoded NBs are amplified with bin-indexed primers prior to NGS analysis. To support statistical analysis of time dependent redistribution between SORS bins, the minimum number of cells bearing a given NB/TPD complex that is sorted at T=0 is about 100.

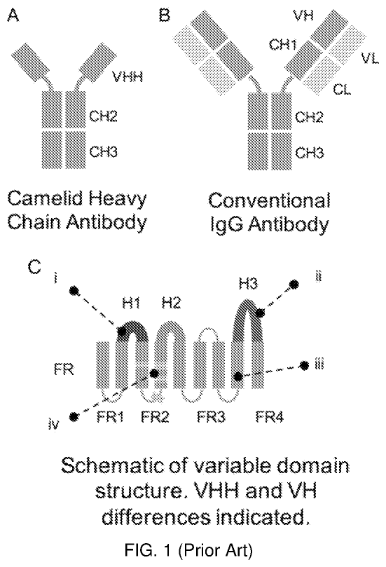

### DETAILED DESCRIPTION

Other than in the operating examples, or where otherwise indicated, the use of numerical values in the various ranges specified in this application are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. Further, as used herein, all numbers expressing dimensions, physical characteristics, processing parameters, quantities of ingredients, reaction conditions, and the like, used in the specification and claims are to be understood as being modified in all instances by the term “about”. Moreover, unless otherwise specified, all ranges disclosed herein are to be understood to encompass the beginning and ending range values and any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more and ending with a maximum value of 10 or less, e.g., 1 to 3.3, 4.7 to 7.5, 5.5 to 10, and the like.

As used herein “a” and “an” refer to one or more. The term “comprising” is open-ended and may be synonymous with “including”, “containing”, or “characterized by”. The term “consisting essentially of” limits the scope of a claim to the specified materials or steps and those that do not materially affect the basic and novel characteristic(s) of the claimed invention.

As used herein, spatial or directional terms, such as “left”, “right”, “inner”, “outer”, “above”, “below”, “over”, “under”, and the like, relate to the invention as it is shown in the drawing figures are provided solely for ease of description and illustration, and do not imply directionality, unless specifically required for operation of the described aspect of the invention. It is to be understood that the invention can assume various alternative orientations and, accordingly, such terms are not to be considered as limiting.

As used herein, a “patient” or “subject” is an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, a horse, and a whale), or a bird (e.g., a duck or a goose). As used herein, the terms “treating”, or “treatment” refer to a beneficial or desired result, such as improving one of more functions, or symptoms of a disease.

Unless stated otherwise, nucleotide sequences are recited herein in a 5′ to 3′ direction, and amino acid sequences are recited herein in an N-terminal to C-terminal direction according to convention.

An antibody is an immunoglobulin molecule produced by B lymphoid cells with a specific amino acid sequence. Antibodies are evoked in humans or other animals, such as camelids, by a specific antigen (immunogen). Antibodies are characterized by reacting specifically with the antigen in some demonstrable way, antibody and antigen each being defined in terms of the other. “Eliciting an antibody response” refers to the ability of an antigen or other molecule to induce the production of antibodies.

An immunogen is a compound, composition, or substance that can stimulate the production of antibodies or a T-cell response in an animal, including compositions that are injected or absorbed into an animal. An antigen traditionally reacts with the products of specific humoral or cellular immunity, including those induced by heterologous immunogens, and in the context of the present disclosure, reacts with naturally-generated, or synthetically mutated, V_{HH} peptides (nanobodies). In the context of the present disclosure an antigen is the described target protein domain (TPD). In one embodiment, a TPD is a fragment of a coronavirus spike protein, such as an ectodomain or fragment thereof or a protein comprising an epitope of a coronavirus protein, such as a spike protein. Other suitable TPDs include, without limitation: cytokines or other secreted proteins, microbe protein fragments or pathogenic microbe protein fragments including ectodomains thereof, cell surface receptor fragments such as ectodomains or ligand binding sites thereof including cancer-associated cell surface markers such as EGFR, parasite protein fragments or pathogenic parasite protein fragments including ectodomains thereof, cell surface receptor fragments such as ectodomains or ligand binding sites thereof. A microbe is a single-cell organism, such as, for example and without limitation, a virus, bacteria, fungus, or protozoa, that may be pathogenic (disease-causing). A parasite may be, for example and without limitation, single-cell as in the exemplary cases of Trypanosome or Plasmodium, multicellular as in the exemplary cases of hookworm and lice, of fungal as in the exemplary case of ringworm.

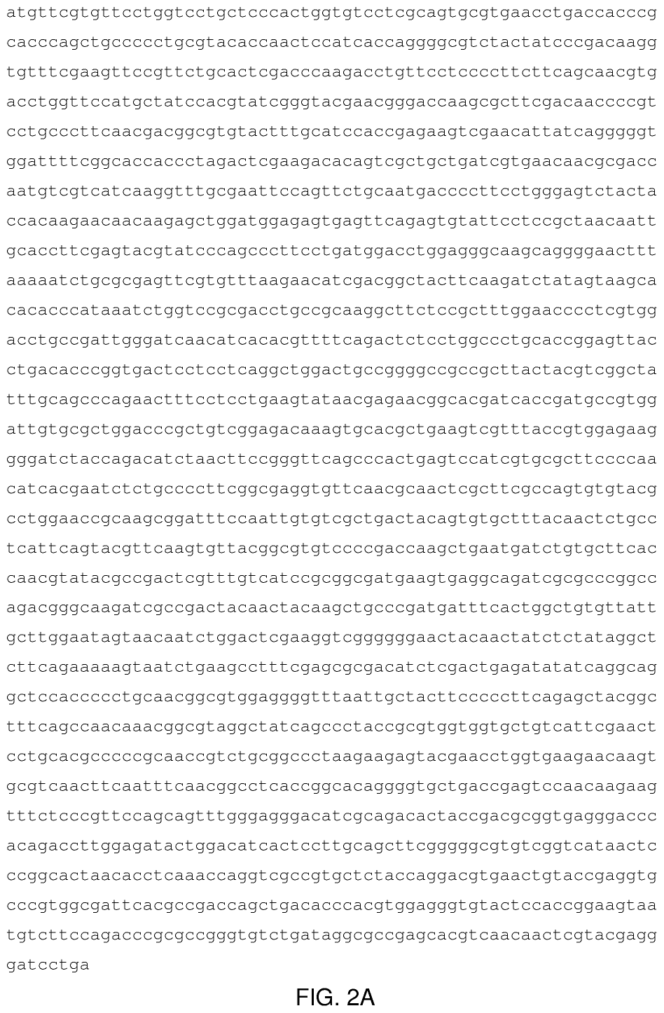

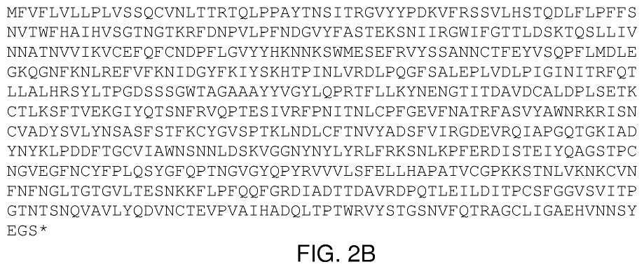

As a non-limiting and purely illustrative example in relation to desirable TPD sequences, coronaviruses (Coronoviridae) are members of the Nidovirales order, and are enveloped, non-segmented positive-sense RNA viruses. The most prominent feature of coronaviruses is the club-shape spike projections emanating from the surface of the virion. These spikes are a defining feature of the virion and give them the appearance of a solar corona, prompting the name, coronaviruses. Homotrimers of the virus encoded S protein make up the distinctive spike structure on the surface of the virus. The trimeric S glycoprotein is a class I fusion protein and mediates attachment to the host receptor. In most, but not all, coronaviruses, S is cleaved by a host cell furin-like protease into two separate polypeptides noted S1 and S2. S1 makes up the large receptor-binding domain of the S protein while S2 forms the stalk of the spike. Examples of sequences of the SARS-CoV-2 spike protein are provided in FIGS. 2A and 2B. SARS-CoV, MERS-CoV, and SARS-CoV-2 are members of the betacoronavirus (or group 2 coronavirus) genera. Coronavirus proteins include, for example and without limitation, and among others: spike (S1 and S2) proteins, M (membrane) protein, N (nucleocapsid) protein, ORFlab (ORFla and ORF1b) proteins, ORF3 proteins, the sequences of which are broadly published and are freely available (See, e.g., NCBI Reference Sequence: NC_045512.2). Suitable TPDs may comprise any full-length or partial amino acid sequences of any coronavirus protein, or an epitope thereof.

A “codon-optimized” nucleic acid refers to a nucleic acid sequence that has been altered such that the codons are optimal for expression in a particular system (such as a particular species of group of species). For example, a nucleic acid sequence can be optimized for expression in yeast cells. Codon optimization does not alter the amino acid sequence of the encoded protein.

A conservative substitution is a substitution of one amino acid residue in a protein sequence for a different amino acid residue having similar biochemical properties. Typically, conservative substitutions have little to no impact on the activity of a resulting polypeptide. For example, a TPD polypeptide sequence may include one or more conservative substitutions (for example 1-10, 2-5, or 10-20, or no more than 2, 5, 10, 20, 30, 40, or 50 substitutions) yet retains the antigenic structure and function of the wild-type protein, namely, in the context of the present disclosure, the ability to bind to a nanobody co-expressed in a yeast cell. A polypeptide can be produced to contain one or more conservative substitutions by manipulating the nucleotide sequence that encodes that polypeptide using, for example, standard procedures such as site-directed mutagenesis or PCR. Methods are provided herein to ascertain proper expression of any TPD sequence.

The term “contacting” refers to placement in direct physical association; includes both in solid and liquid form. “Contacting” is often used interchangeably with “exposed.” In some cases, “contacting” includes transfecting, such as transfecting a nucleic acid molecule into a cell. In other examples, “contacting” refers to incubating a molecule (such as an antibody) with a biological sample.

A fusion protein or fusion polypeptide refers to a protein or polypeptide generated, for example, by expression of a nucleic acid sequence engineered from nucleic acid sequences encoding at least a portion of two different (heterologous) proteins. To create a fusion protein, the nucleic acid sequences are in the same reading frame and contain no internal stop codons. For example, as described in further detail herein, a fusion protein includes a TPD fused to an FAP, or a nanobody fused to a FAP. The FAP fused to the TPD may be different from the FAP fused to the nanobody in that they bind different fluorogens having different emission spectra when bound by their cognate FAP.

An epitope is an antigenic determinant capable of inducing humoral and/or cell-mediated immune response or immunity, that is the portion of an antigen that is recognized by the immune system, and in the case of a protein antigen, comprises a specific amino acid sequence. Epitopes may be identified by any useful epitope mapping method, e.g., as are broadly-known in the arts.

An epitope string is an engineered polypeptide comprising linked epitopes of any one or more proteins. For example, an epitope may be identified within a full protein sequence, and amino acids between epitopes may be deleted partially or fully. The epitope string need only function as a TPD, and therefore structure and function of the protein from which the epitope is derived need not be conserved, permitting significant flexibility in inter-epitope deletions, combinations of epitopes from multiple proteins, and/or substitutions in the spacer sequence between the defined epitopes. For example, and epitope string may be produced by deleting one or more amino acids from a native amino acid sequence, and/or combining epitopes, and optionally flanking sequences in frame from multiple native proteins, comprising epitopes including flanking (e.g., from six to ten) native N-terminal and C-terminal amino acids as spacers.

It would be recognized that the nature of the TPDs described herein and the described methods do not require that the TPD comprises a recognized camelid or other species' epitope. Nevertheless, and solely for illustration, with regard to coronavirus spike protein-based TPDs, Ferretti A P, et al. (Ferretti A P, et al. Unbiased Screens Show CD8+ T Cells of COVID-19 Patients Recognize Shared Epitopes in SARS-CoV-2 that Largely Reside Outside the Spike Protein. Immunity. 2020-11-17; 53 (5): 1095-1107.e3) provides an exemplary list of CD8+ T cell epitope sequences identified in SARS-CoV-2 sequences that may be used as TPDs or as parts of TPDs for the methods herein. An epitope string may comprise one or more repeats of a single epitope amino acid sequence, and/or may comprise multiple different amino acid sequences.

An immunogen refers to a compound, composition, or substance which is capable, under appropriate conditions, of stimulating an immune response, such as the production of antibodies, such as neutralizing antibodies, or a T-cell response in an animal, including compositions that are injected or absorbed into an animal. As used herein, an “immunogenic composition” is a composition comprising an immunogen (such as a fragment of a cancer-associated surface marker or viral ectodomain or an epitope string comprising two or more epitopes), which may have the same or different amino acid sequences. Immunogens may be used to elicit an immune response in a camelid to enrich V_{HH} sequences specific to a desired target protein. Multivalent V_{HH} sequences can be obtained, for example from RNA of peripheral blood mononuclear cells (PBMCs), by standard methodologies, including, for example, a reverse transcription step followed by PCR amplification of Vin sequences, which may be inserted into a suitable vector of propagation and further cloning, for example as described herein.

An “isolated” or “purified” biological component (such as a nucleic acid, peptide, protein, protein complex, or particle) refers to a component that has been substantially separated, produced apart from, or purified away from other components in a preparation or other biological components in the cell of the organism in which the component occurs, that is, other chromosomal and extrachromosomal DNA and RNA, and proteins. Nucleic acids, peptides and proteins that have been “isolated” or “purified”, thus, include, for example and without limitation, nucleic acids and proteins purified by standard purification methods. The term also embraces nucleic acids, peptides and proteins prepared by recombinant expression in a host cell, as well as chemically synthesized nucleic acids or proteins. The term “isolated” or “purified” does not require absolute purity; rather, it is intended as a relative term. Thus, for example, an isolated biological component is one in which the biological component is more enriched than the biological component is in its natural environment within a cell, or other production vessel. A preparation may be purified such that the biological component represents at least 50%, such as at least 70%, at least 90%, at least 95%, or greater, of the total biological component content of the preparation.

A linker refers to a molecule or group of atoms positioned between two moieties (portions of a molecule). Linkers may be bifunctional, e.g., the linker includes a functional group at each end, wherein the functional groups are used to couple the linker to the two moieties. The two functional groups may be the same, e.g., a homobifunctional linker, or different, e.g., a heterobifunctional linker. A peptide linker or spacer may be used to link the C-terminus of a first polypeptide to the N-terminus of a second polypeptide in a fusion protein or peptide. Non-limiting examples of peptide linkers or spacers include glycine-serine peptide linkers. Typically, such linkage is accomplished using molecular biology techniques to genetically manipulate DNA encoding the first polypeptide linked to the second polypeptide by the peptide linker in an open reading frame. Spacers or linkers are common to fusion proteins, and may be inserted between subunits of the fusion protein as described herein. Use and characterization of spacer amino acid sequences, is routine, and choice of which is well within the abilities of a person of ordinary skill in the art. For example and without limitation, spacer amino acids may include polar uncharged or charged amino acids. Peptide linkers or spacers may include amino acids, e.g. of from 1 to 50 amino acids in length, corresponding to flanking polypeptides naturally present in a polypeptide included in the described fusion proteins, such as sequences flanking a VHH peptide. Spacers may be of sufficient length and rigidity, e.g. forming an alpha helix, such that functional elements of the fusion protein are sufficiently separated to maintain their desired functionality, such as the FAP, NB, and target antigen portions of the fusion proteins (See, e.g., Chen X, et al. Fusion protein linkers: property, design and functionality. Adv Drug Deliv Rev. 2013 October; 65 (10): 1357-69). Spacers may have a length ranging from 1-100 Å (Angstroms), or from 1 to 50 amino acids. As above, codon degeneracy and choice of amino acid for spacers may be unique to each clone, such that the DNA sequence acts as a barcode.

A nucleic acid molecule (a nucleic acid) refers to a polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” The term includes single- and double-stranded forms of DNA. A polynucleotide may include either or both naturally occurring and modified nucleotides linked together by naturally occurring and/or non-naturally occurring nucleotide linkages.

A first nucleic acid is said to be operably linked to a second nucleic acid when the first nucleic acid is placed in a functional relationship with the second nucleic acid. Generally, operably linked DNA sequences are contiguous (e.g., in cis) and, where the sequences act to join two protein coding regions, in the same reading frame (e.g., open reading frame or ORF), for example to produce a fusion protein. Operably linked nucleic acids include a first nucleic acid contiguous with the 5′ or 3′ end of a second nucleic acid. In other examples, a second nucleic acid is operably linked to a first nucleic acid when it is embedded within the first nucleic acid, for example, where the nucleic acid construct includes (in order) a portion of the first nucleic acid, the second nucleic acid, and the remainder of the first nucleic acid.

A polypeptide is a polymer in which the monomers are amino acid residues which are joined together through amide bonds. When the amino acids are alpha-amino acids, either the L-optical isomer or the D-optical isomer can be used. The terms “polypeptide”, “peptide”, or “protein” as used herein are intended to encompass any amino acid sequence and include proteins and modified sequences such as glycoproteins. The term “polypeptide” is specifically intended to cover naturally occurring proteins, as well as those which are recombinantly or synthetically produced. The term “residue” or “amino acid residue” includes reference to an amino acid that is incorporated into a protein, polypeptide, or peptide.

Conservative amino acid substitutions are those substitutions that, when made, least or minimally interfere with the properties of the original protein, that is, in the context of the end-use, the structure and function of the protein is conserved and not significantly changed by such substitutions, and may be identified by use of matrices, such as the BLOSUM series of matrices, and other matrices. Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be non-conservative, for instance changes in which (a) a hydrophilic residue, for example, seryl or threonyl, is substituted for (or by) a hydrophobic residue, for example, leucyl, isoleucyl, phenylalanyl, valyl, or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

A promoter is an array of nucleic acid control sequences which direct transcription of a nucleic acid. A promoter includes necessary nucleic acid sequences near the start site of transcription. A promoter also optionally includes distal enhancer or repressor elements. A “constitutive promoter” is a promoter that is continuously active and is not subject to regulation by external signals or molecules. In contrast, the activity of an “inducible promoter” is regulated by an external signal or molecule (for example, a transcription factor). A bicistronic promoter initiates the production of two mRNAs, as with the GAL1/10 UAS promoter described in further detail herein, or a single transcript that is translated to produce two or more proteins, as with an IRES element, or the inclusion of a 2A self-cleaving peptide in a fusion protein.

A recombinant nucleic acid refers to a nucleic acid molecule (or protein or virus) that is not naturally occurring or has a sequence that is made by an artificial combination of two otherwise separated segments of sequence. This artificial combination is accomplished by chemical synthesis or, more commonly, by the artificial manipulation of isolated segments of nucleic acids, e.g., by genetic engineering techniques such as those described in Sambrook et al., (ed.), Molecular Cloning: A Laboratory Manual, 2nd ed., vol. 1-3, Cold Spring Harbor Laboratory Press, Cold Spring Harbor, N.Y., 1989. The term recombinant includes nucleic acids and proteins that have been altered solely by addition, substitution, or deletion of a portion of a natural nucleic acid molecule or protein.

“Sequence identity” refers to the similarity between nucleic acid or amino acid sequences is expressed in terms of the similarity between the sequences, otherwise referred to as sequence identity. Sequence identity may be measured in terms of percentage identity (or similarity or homology); the higher the percentage, the more similar the two sequences are. Homologs, orthologs, or variants of a polypeptide will possess a relatively high degree of sequence identity when aligned using standard methods. Methods of alignment of sequences for comparison are well-known in the art. Various programs and alignment algorithms are described in the art, for example, see: Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Corpet et al., Nuc. Acids Res. 16:10881-90, 1988; and Pearson et al., Meth. Mol. Bio. 24:307-31, 1994. Altschul et al., J. Mol. Biol. 215:403-10, 1990, presents a detailed consideration of sequence alignment methods and homology calculations.

Once aligned, the number of matches may be determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a peptide sequence that has 1166 matches when aligned with a test sequence having 1554 amino acids is 75.0 percent identical to the test sequence (1166-1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer.

Homologs and variants of a polypeptide are typically characterized by possession of at least about 75%, for example, at least about 80%, 85%, 90%, 91%, 92%, 93%, 94%, 95%, 96%, 97%, 98%, or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, for example and without limitation, by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection. One example of a useful algorithm is PILEUP. PILEUP uses a simplification of the progressive alignment method of Feng & Doolittle, J. Mol. Evol. 35:351-360, 1987. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nucl. Acids Res. 12:387-395, 1984).

Another example of algorithms that are suitable for determining percent sequence identity and sequence similarity are the BLAST and the BLAST 2.0 algorithm, which are described in Altschul et al., J. Mol. Biol. 215:403-410, 1990 and Altschul et al., Nucleic Acids Res. 25:3389-3402, 1977. Software for performing BLAST analyses is publicly available through the National Center for Biotechnology Information (ncbi.nlm.nih.gov). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see, Henikoff & Henikoff, Proc. Natl. Acad. Sci. USA 89:10915, 1989). An oligonucleotide is a linear polynucleotide sequence of up to about 100 nucleotide bases in length.

As used herein, reference to “at least 80% identity” (or similar language) refers to “at least 80%, at least 85%, at least 90%, at least 91%, at least 92%, at least 93%, at least 94%, at least 95%, at least 96%, at least 97%, at least 98%, at least 99%, or even 100% identity” to a specified reference sequence. As used herein, reference to “at least 90% identity” (or similar language) refers to “at least 90%, at least 91%, at least 92%, at least 93%, at least 94%, at least 95%, at least 96%, at least 97%, at least 98%, at least 99%, or even 100% identity” to a specified reference sequence.

Complementary refers to the ability of polynucleotides (nucleic acids) to hybridize to one another, forming inter-strand base pairs. Base pairs are formed by hydrogen bonding between nucleotide units in polynucleotide strands that are typically in antiparallel orientation. Complementary polynucleotide strands can base pair (hybridize) in the Watson-Crick manner (e.g., A to T, A to U, C to G), or in any other manner that allows for the formation of duplexes. In RNA as opposed to DNA, uracil rather than thymine is the base that is complementary to adenosine. Two sequences comprising complementary sequences can hybridize if they form duplexes under specified conditions, such as in water, saline (e.g., normal saline, or 0.9% w/v saline) or phosphate-buffered saline), or under other stringency conditions, such as, for example and without limitation, 0.1×SSC (saline sodium citrate) to 10×SSC, where 1×SSC is 0.15M NaCl and 0.015M sodium citrate in water. Hybridization of complementary sequences is dictated, e.g., by the nucleobase content of the strands, the presence of mismatches, the length of complementary sequences, salt concentration, temperature, with the melting temperature (Tm) lowering with shorter complementary sequences, increased mismatches, and increased stringency. Perfectly matched sequences are said to be “fully complementary”, though one sequence (e.g., a target sequence in an mRNA) may be longer than the other.

A “transformed” cell is a cell into which has been introduced a nucleic acid molecule (such as a heterologous nucleic acid) by any useful molecular biology technique. The term encompasses all techniques by which a nucleic acid molecule might be introduced into such a cell, including, for example and without limitation, transfection with viral vectors, transformation with plasmid vectors, and introduction of naked DNA by electroporation, lipofection, or particle gun acceleration.

A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. An insertional vector is capable of inserting itself into a host nucleic acid. A vector can also include one or more selectable marker genes and other genetic elements. An expression vector is a vector that contains the necessary regulatory sequences to allow transcription and translation of inserted gene or genes.

By “expression” or “gene expression,” it is meant the overall flow of information from a gene or functional/structural RNA, and a polyadenylation sequence), to produce a gene product (typically a protein, optionally post-translationally modified or a functional/structural RNA). A “gene” refers to a functional genetic unit for producing a gene product, such as RNA or a protein in a cell, or other expression system encoded on a nucleic acid and comprising: a transcriptional control sequence, such as a promoter and other cis-acting elements, such as transcriptional response elements (TREs) and/or enhancers; an expressed sequence that may encode a protein (referred to as an open-reading frame or ORF), and a polyadenylation sequence. By “expression of genes under transcriptional control of,” or alternately “subject to control by,” a designated sequence such as TRE or transcription control element, it is meant gene expression from a gene containing the designated sequence operably linked (functionally attached, typically in cis) to the gene. A “gene for expression of” a stated gene product is a gene capable of expressing that stated gene product when placed in a suitable environment—that is, for example, when transformed, transfected, transduced, etc. into a cell, and subjected to suitable conditions for expression. In the case of a constitutive promoter “suitable conditions” means that the gene typically need only be introduced into a host cell. In the case of an inducible promoter, “suitable conditions” means when factors that regulate transcription, such as DNA-binding proteins, are present or absent—for example an amount of the respective inducer is available to the expression system (e.g., cell), or factors causing suppression of a gene are unavailable or displaced-effective to cause expression of the gene.

A coronavirus polypeptide is a polypeptide encoded by a coronavirus or a portion of a polypeptide encoded by a coronavirus, as in epitopes and immunogenic fragments thereof, and polypeptides with significant sequence identity (e.g., at least 85%, 90%, 95%, 98%, or 99%) with a polypeptide encoded by a natural coronavirus, and retaining or improving upon native function as an immunogen, epitope, or TPD in the case of the present disclosure.

Unless otherwise explained, all technical and scientific terms used herein have the same meaning as commonly understood by one of ordinary skill in the art to which this disclosure belongs. It is to be understood that all base sizes or amino acid sizes, and all molecular weight or molecular mass values, given for nucleic acids or polypeptides are approximate, and are provided for description. Unless otherwise indicated, polymer molecular weight is expressed as number-average molecular weight (Mn). Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

TPDs are disclosed herein. These TPDs may be used in the described vectors, fusion proteins, cells, and methods to determine quantitatively or qualitatively the binding of nanobodies to the TPD. The TPD may be randomized fragments of a larger protein, an entire protein, or selected fragments of a desired protein. The TPD may comprise an amino acid sequence of any protein of biological or therapeutic relevance, such as a receptor or cell marker associated with a cancer, a cell marker characteristic of an infection, or a pathogen. In one non-limiting example, the TPD may be a fragment of a coronavirus S protein, e.g. a fragment of an ectodomain of a Coronavirus S protein. The coronavirus may be SARS-CoV-2. The TPD may be part of, or a complete cytokine, or a receptor ectodomain. For example, the TPD may be a fragment of an ectodomain of EGFR or PD1. It is noted that sequences of SARS-CoV-2 are provided herein as merely illustrative of proteins that include amino acid sequences of potential TPD peptides. Databases, such as GenBank and UniProt, as well as the literature, are replete with proteins as well as protein structures, that can provide suitable candidate TPD peptides for analysis provided herein.

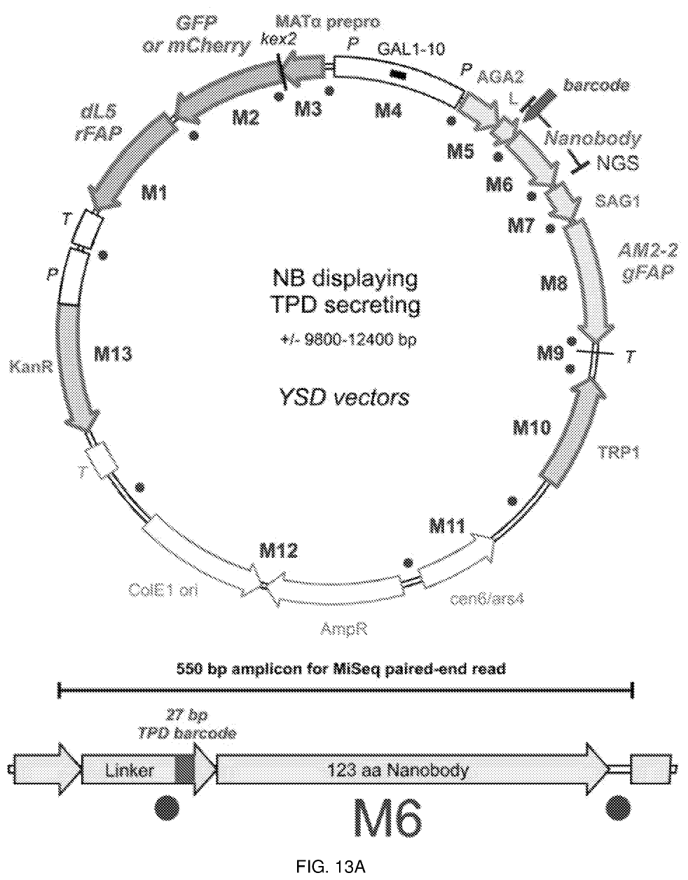

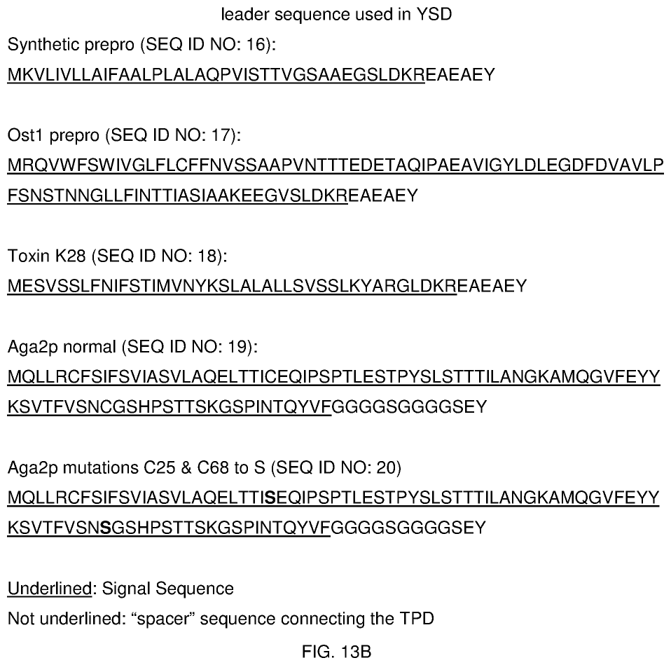

Additional amino acid sequences for inclusion in the described fusion proteins, include, for example and without limitation, linkers, spacers, carriers (see, e.g., US 2020/0031874, incorporated herein by reference), signal peptides, self-cleaving sequences, and affinity tags, may be included in the fusion protein, so long as they do not interfere to any significance with the operation of the fusion proteins as described herein. Secretion peptides for incorporation into the fusion proteins may include, for example and without limitation: synthetic prepro (most important), α-mating factor secretion domain (α-MF), Toxin K28, AGA2p, Aga2p mut C25/68 to S and OST1 leader, e.g. as shown in FIG. 13B. The SAG1 sequences are from the heavily mannosylated ‘stalk’ portion of a yeast cell surface protein involved in mating agglutination. This segment serves to separate the TPD and FAP (thereby reducing opportunities for steric interference) and has been reported to increase expression levels on the yeast surface. Most yeast cell surface proteins have a GPI anchor/stalk/globular domain architecture where the globular region confers the relevant surface properties. Including the SAG1 stalk with our globular TPDs makes our secreted proteins structurally mimic typical yeast cell surface proteins. The SAG1 stalk doesn't promote cell-cell agglutination but the SAG1 globular domain does. A non-limiting example of a surface display peptide is Aga2, as shown herein.

Nucleic acids and vectors encoding the described fusion proteins may be provided. In some non-limiting examples, disclosed is a recombinant vector, such as a yeast plasmid, that expresses the disclosed fusion proteins. One of skill in the art can readily use the genetic code to construct a variety of functionally equivalent nucleic acids, such as nucleic acids which differ in sequence but which encode the same protein sequence due to codon degeneracy. In some embodiments, the polynucleotide is codon-optimized for expression in yeast cells.

Exemplary nucleic acids may be prepared by cloning techniques, e.g., as are broadly-known and implemented either commercially, or in the art. Multiple textbooks and reference manuals describe and provide examples of useful and appropriate cloning and sequencing techniques, and instructions sufficient to direct persons of skill through such techniques are known. Commercial and public product information from manufacturers of biological reagents and experimental equipment also provide useful information. Such manufacturers include the SIGMA Chemical Company (Saint Louis, Mo.), R&D Systems (Minneapolis, Minn.), Pharmacia Amersham (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemic AG, Buchs, Switzerland), Invitrogen (Carlsbad, Calif.), Addgene, and Applied Biosystems (Foster City, Calif.), as well as many other commercial sources.

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

A streamlined recombinant workflow may be preferred, where recombinant procedures are carried out in yeast using homologous recombination of amplicons, although shuttle vectors may support engineering in E. coli if desired. A synthetic biology amplicon-based approach is especially suited to constructing TPD query sets, where bioinformatics can be applied to design TPDs on the basis of domain structure, splicing junctions, point mutations, yeast codon optimization, and many other biological considerations. In the methods below, there is no need to purify and characterize protein. The yeast secretory quality control apparatus can be used to ensure that the displayed NBs and secreted TPDs are expressed and properly folded. The fluorescence of downstream FAP tags can be used as a proxy that provides simple, screenable assays.

The nucleic acids for expressing the disclosed fusion proteins can include a recombinant DNA which is incorporated into an autonomously replicating plasmid or into the genomic DNA of a yeast cell. The term includes single- and double-stranded forms of DNA.

Nucleic acid sequences encoding a disclosed fusion protein sequence can be operatively linked to expression control sequences. An expression control sequence operatively linked to a coding sequence is placed in the sequence such that expression of the coding sequence is achieved under conditions compatible with the expression control sequences. The expression control sequences include, but are not limited to, appropriate promoters, enhancers, transcription terminators, a start codon (e.g., ATG) in front of a protein-encoding gene, splicing signal for introns, maintenance of the correct reading frame of that gene to permit proper translation of mRNA, and stop codons.

Hosts can include yeast cells, such as, for example and without limitation, Pichia pastoris, Saccharomyces cerevisiae, Kluyveromyces lactis, or Hansenula polymorpha cells. Methods for growth, storage, transformation, and propagation of yeast cells are broadly-known. Transformation of a host cell with recombinant DNA can be carried out by conventional techniques as are well known to those skilled in the art. Such methods of transfection of DNA include calcium phosphate co-precipitates, conventional mechanical procedures such as microinjection, electroporation, insertion of a plasmid encased in liposomes, or viral vectors can be used. One of skill in the art can readily use expression system such as plasmids and vectors for use in producing proteins in yeast cells.

As described herein, “yeast” is a single-celled, eukaryotic microbe that can grow quickly in complex or defined media with doubling times typically around 2.5 h in glucose-containing media. Yeast is easier and less expensive to use for recombinant protein production than insect or mammalian cells. The most commonly employed yeasts in the laboratory are Saccharomyces cerevisiae and certain members of the Pichia genus, such as Pichia pastoris (Komagataella phaffii). While S. cerevisiae may be commonly used, and used in the examples herein, other yeasts may be effectively employed in the systems and methods described herein. Other exemplary yeasts include Kluyveromyces lactis and Hansenula polymorpha. In one example, the yeast cell is S. cerevisiae strain EBY100, which is broadly-available, e.g., Saccharomyces cerevisiae Meyen ex E. C. Hansen, ATCC accession No. MYA-4941.

A polymer “comprises” or is “derived from” a stated monomer if that monomer is incorporated into the polymer. Thus, the incorporated monomer (monomer residue) that the polymer comprises is not the same as the monomer prior to incorporation into a polymer, in that at the very least, certain groups/moieties are missing and/or modified when incorporated into the polymer backbone. A polymer is said to comprise a specific type of linkage if that linkage is present in the polymer, such as, without limitation: ester, amide, carbonyl, ether, thioester, thioether, disulfide, sulfonyl, amine, carbonyl, phosphodiester, or carbamate bonds. As such, peptides comprise amino acid residues, and nucleic acids comprise nucleotide residues.

Nanobodies (NBs) have become the predominant small immunoreagent in research, biotechnology, and medicine. Although classical antibodies have been mainline reagents and therapeutics for over 30 years. NBs are (1) smaller and simpler which makes them easy to recombinantly engineer; (2) generally free from autoimmune reactions that compromise natural antibodies; and (3) can be inexpensively and rapidly synthesized without host animals.

This NB discovery technology represents a breakthrough in NB screening methodology and in how these immunoreagents will be used in future applications, as our system can be used in a ‘many-on-many’ NB screening mode, without physically purifying the NBs and Target Protein domains (TPDs). In a major advance, we demonstrated in a model system that our high-throughput yeast secrete and display (YSD) platform enables the isolation and identification, using next generation sequencing (NGS), of surface-displayed NBs that specifically bind secreted cognate TPDs. To mitigate difficulties in obtaining target-specific nanobodies: (1) A FACS reporter assay is provided that quantitatively reports the interaction of co-expressed NB and target protein domains (TPDs) in terms of specificity, affinity and kinetics, thus avoiding the use of purified protein; (2) a method is provided that physically fuses the encoded genotypes of co-expressed NB and TPD, enabling next generation sequencing (NGS) analysis to resolve FACS assays of complex populations into the binding phenotypes of individual clones, thus eliminating the need for physical cloning; and (3) multiplexed screens are provided in the form of bioinformatics-derived TPD query sets to directly obtain groups of related reagents, thus minimizing the need to evaluate individual clones in isolation. These methods may be integrated into a simple and inexpensive yeast platform for the high throughput creation of NB toolkits with expanded applications and provides small recombinant protein affinity reagents for biology and medicine.

In further detail, the technology presented here is a unique collection of small fluorogen activating proteins (FAPs) and cognate fluorogen chemistries. The NB discovery platform concept is based on a dual self-reporting fluorogenic reporter system within a single yeast cell that supports parallel, multiplexed and, above all, combinatorial NB screening capabilities. The fluorogenic TDs can be used in a non-reversible or reversible mode to stabilize a cell surface complex of a secreted target protein and a displayed cognate probe protein. Sequential screens, first using a non-reversible TD, and then using MG-PEG plus TO1-PEG, comprise a basic secrete-and-capture system. In and of itself, this is a multidimensional addition to yeast surface co-expression technologies. This basic system can be directly used with current FACS screens to isolate and improve NBs and other small affinity reagent proteins. Furthermore, this system is the genesis of new ways to study the secretion dynamics of TPDs or other proteins of interest by enabling specific targeting of different yeast cell envelope compartments (periplasmic space, inner cell wall (glycans) and outermost cell surface (mannoproteins)) by employing a series of low to high MW PEGylated fluorogens. The novel GG YSD vector platform is employed to fuse, by mass action, sequences encoding probe and target to create NB/TPD libraries without loss of complexity. This technical advance allows high throughput combinatorial NGS analysis of complex diverse NB/TPD populations and eliminates the need to isolate and evaluate individual clones. Such a paradigm shift in screening technology also gives rise to a shift in the kind of targets for which cognate NBs may be readily isolated. Bioinformatics can be used to design TPD query sets to directly isolate NBs that probe biological functionalities that were previously very difficult to approach. Queries will also enable the concerted production of NB toolkits tailored to address biological and therapeutic themes of interest.

A fluorogen activating protein (FAP) is an activator polypeptide that selectively binds a fluorogen and increases fluorescent emission from the fluorogen. As such, a FAP is a binding partner of its cognate fluorogen (fluorophore). In the examples below, the fluorophores malachite green (MG, a triaryl methine dye, see, e.g., U.S. Pat. No. 9,249,306, incorporated herein by reference) and thiazole orange (e.g., sulfonated TO1, as shown below). The fluorogen may have an excitation wavelength such that when bound by the FAP activator and exposed to light at the excitation wavelength the fluorogen produces a detectably different, and in one instance increased, fluorescent emission as compared to unbound fluorogen exposed to light at the same excitation wavelength. The excitation light can be produced by any light-emitting device, such as a lamp, a light-emitting diode, or a laser, as are broadly known by those of skill in the art. International Patent Publication No. WO 2008/092041, incorporated herein by reference in its entirety, describes and provides amino acid sequences of suitable FAPs for binding MG moieties and TO1 or other monomethine cyanine dye moieties. Also described in WO 2008/092041 are PEG-modified versions of MG and TO1.

In further detail, fluorogen activating proteins (FAPs) are a class of fluorescence-based molecular tags that have been used in a variety of trafficking assays due to the rapid noncovalent association and activation of a fluorogenic dye by the expressed protein tag. One FAP can exhibit distinct properties when combined with various dye derivatives. FAPs may be derived from single chain variable fragment antibodies (scFv) that specifically recognize and activate fluorogenic dyes with high binding affinity and provide modularity in targeted labeling without the need for direct conjugation of dyes. Fluorogens useful in combination with FAPs are organic dyes that have low fluorescence signal when free in solution and show significantly enhanced fluorescence output upon binding to the FAP. These dye-FAP fluoromodules show several advantages as fluorescent tags in cell biological studies. FAPs are small expressible protein modules that have typically been cloned as a fusion to proteins of interest. Work has demonstrated the use as recombinant affinity tags for secondary detection of fluorescein (Saunders, M. J., Block, E., Sorkin, A., Waggoner, A. S., and Bruchez, M. P. (2014) A bifunctional converter: fluorescein quenching scFv/fluorogen activating protein for photostability and improved signal to noise in fluorescence experiments. Bioconjugate Chem. 25 (8), 1556-1564) or biotin modified proteins (Gallo, E., and Jarvik, J. (2014) Fluorogen-activating scFv biosensors target surface markers on live cells via streptavidin or single-chain avidin. Mol. Biotechnol. 56, 585-590). The fast association and activation of fluorogen/FAP complexes shortens the time for labeling protocols. Since the fluorescence is dependent on the association of fluorogen to the FAP, the fluoromodules allow order-of-addition and compartment selectivity to achieve subpopulation labeling for internalized receptors. A number of modifications to fluorogens enrich the functional properties of a single FAP for optical labeling, such as membrane permeability/exclusion (Szent-Gyorgyi, C., et al. (2008) Fluorogen-activating single-chain antibodies for imaging cell surface proteins. Nat. Biotechnol. 26, 235-240), fluorescence brightness (Szent-Gyorgyi, C., et al. (2010) Fluorogenic dendrons with multiple donor chromophores as bright genetically targeted and activated probes. J. Am. Chem. Soc. 132, 11103-11109), and environmental sensitivity (Grover, A., et al. (2012) Genetically encoded pH sensor for tracking surface proteins through endocytosis. Angew. Chem., Int. Ed. Engl. 51, 4838-4842 and Szent-Gyorgyi, C., et al. (2010) J. Am. Chem. Soc. 132, 11103-11109). Thus, fusion of a nanobody or target antigen to a FAP should provide a compact probe and tethering mechanism to instantaneously label and bind nanobody paratopes with their cognate epitopes in the screening process described herein.

Several FAPs have been reported to activate fluorescence of malachite green (MG) and thiazole orange (TO) derivatives, as well as dimethylindole red (DIR), oxazolethiazole-blue (OTB), and various derivatives of these dyes, resulting in a range of fluoromodules with excitation/emission properties at any desired laser wavelength and emission range, typically with affinities for fluorogens in the low nanomolar to picomolar range (Szent-Gyorgyi, C., et al. (2008) Nat. Biotechnol. 26, 235-240; Ozhalici-Unal, H., et al. (2008) A rainbow of fluoromodules: a promiscuous scFv protein binds to and activates a diverse set of fluorogenic cyanine dyes. J. Am. Chem. Soc. 130, 12620-12621; Senutovitch, N., et al. (2012) A variable light domain fluorogen activating protein homodimerizes to activate dimethylindole red. Biochemistry 51, 2471-2485; Zanotti, K. J., et al. (2011) Blue fluorescent dye-protein complexes based on fluorogenic cyanine dyes and single chain antibody fragments. Org. Biomol. Chem. 9, 1012-1020; Wu Y, et al. Discovery of Small-Molecule Nonfluorescent Inhibitors of Fluorogen-Fluorogen Activating Protein Binding Pair. J Biomol Screen. 2016 January; 21 (1): 74-87).

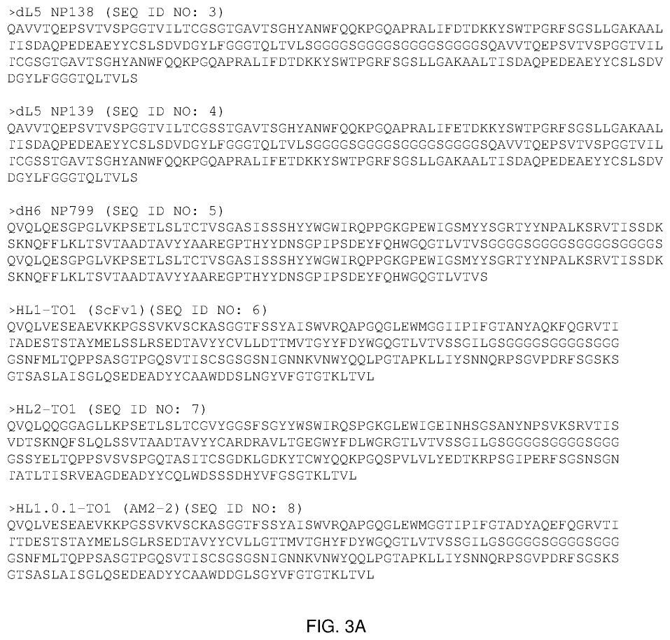

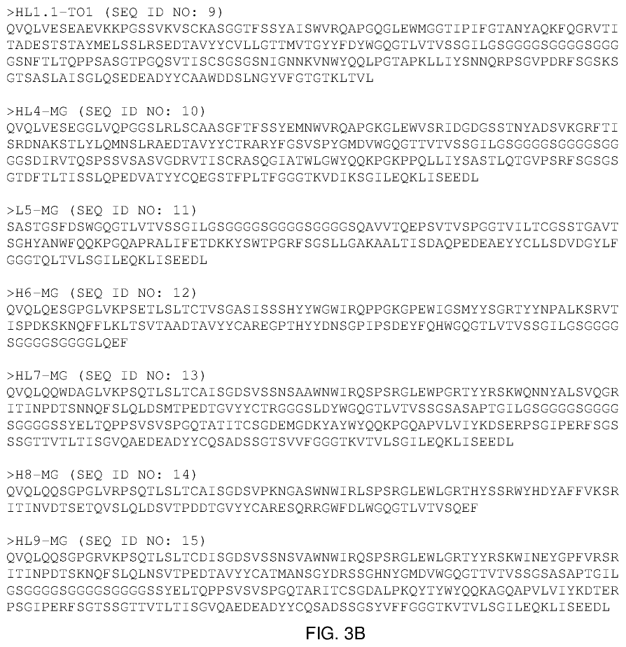

FIGS. 3A and 3B provide non-limiting examples of FAP amino acid sequences that bind triaryl methine dyes, such as malachite green dyes, and monomethine cyanine dyes, such as thiazole orange dyes. As an example, dL5 is relatively small (24.2 kDa), and binds to MG with a low picomolar equilibrium dissociation constant (Szent-Gyorgyi, C., et al. (2013) Malachite green mediates homodimerization of antibody VL domains to form a fluorescent ternary complex with singular symmetric interfaces. J. Mol. Biol. 425, 4595-4613). The extremely low unbound fluorescence background and the high fluorescent signal upon binding allow high contrast and no-wash signal detection (Scc, e.g., Wang Y, et al. Fluorogen activating protein-affibody probes: modular, no-wash measurement of epidermal growth factor receptors. Bioconjug Chem. 2015 Jan. 21; 26 (1): 137-44).

Heavy-chain only antibodies (HcAbs) are found in camelids, such as llamas and alpacas. Compared to conventional mAbs, HcAbs consist of just two heavy chains, with a single variable domain (V_{HH}, ˜15 kDa) as the antigen-binding region. These nanoscale V_{HH}s also may be referred to as “nanobodies” (NBs), retaining full antigen-binding potential upon isolation, establishing them as the smallest, naturally-derived antigen-binding fragment. Nanobodies have spurred the development of commercial companies and have been used in applications such as biosensing, affinity-capture, and protein crystallization; however, their most significant potential lies in therapeutics, e.g., for cancer (See, e.g., Mitchell, L S, et al. Proteins. 2018; 86:697-706 and Yang E Y, Shah K. Nanobodies: Next Generation of Cancer Diagnostics and Therapeutics. Front Oncol. 2020 Jul. 23; 10:1182. doi: 10.3389/fonc.2020.01182).

Unlike other antibody fragments, nanobodies do not require extensive assembly or molecular optimization to create complex constructs and can be readily incorporated as functional subunits of a wide array of nanobody-fusion molecules, including, without limitation: bivalent, biparatopic, bispecific, NB-scFv, NB-cytokine, NB-fluorophore, and NB-drug fusion proteins, as well as NB-nanoparticle and NB-virus complexes. In general, NB antigen specificity is determined at the exposed ends of each variable domain through three peptide loops, or complementarity determining regions (CDRs). The CDR3 loop provides a significant contribution to an antibody's specificity and diversity, and on average, nanobodies have a much greater CDR3 length compared to that of human VH domains, which strengthens their interactions with target antigens (Desmyter A, et al. Antigen specificity and high affinity binding provided by one single loop of a camel single-domain antibody. J Biol Chem. 2001-7-13; 276 (28): 26285-90. doi: 10.1074/jbc.M102107200). The NB CDR3 regions can form finger-like projections that enable high-affinity binding to traditionally inaccessible cavity-like epitopes (De Genst E, et al. Molecular basis for the preferential cleft recognition by dromedary heavy-chain antibodies. Proc Natl Acad Sci USA. (2006) 103:4586-91). The CDR1 and CDR2 regions also aid in antigen binding, which enables greater paratope diversity than that of mAbs (Mitchell L S, et al. Protein Eng Des Sel. 2018-7-1; 31 (7-8): 267-275).

Development and production of nanobodies may be performed commercially, such as by Abcore, Inc. of Ramona, CA and the VIB Nanobody Core at Vrije Universiteit Brussel, Belgium, among others, and numerous patents are directed to nanobody technology, or immunoglobulin single variable domain proteins, for example as illustrated in International Patent Application Publication No. WO 2012175741. Commercial providers provide a range of products ranging from camelid (e.g., llama) blood, PBMCs, RNA or cDNA, a library of nanobody clones, as well as screened clones and large-scale production of positive clones.

Cloning of nanobodies, and modification of nanobodies to improve affinity are broadly-described (See, for example and without limitation: Itoh K. Sokol S Y. Expression cloning of camelid nanobodies specific for Xenopus embryonic antigens. PLoS One. 2014-10-6; 9 (10): e107521; Pardon E, et al., A general protocol for the generation of Nanobodies for structural biology. Nat Protoc. 2014 March; 9 (3): 674-93; Muyldermans S. A guide to: generation and design of nanobodies. FEBS J. 2021 April; 288 (7): 2084-2102; Vincke C. et al. Generation of single domain antibody fragments derived from camelids and generation of manifold constructs. Methods Mol Biol. 2012; 907:145-76; Uchański, T., et al. An improved yeast surface display platform for the screening of nanobody immune libraries. Sci Rep 9, 382 (2019); Güttler T, et al. Neutralization of SARS-CoV-2 by highly potent, hyperthermostable, and mutation-tolerant nanobodies. EMBO J. 2021-10-1; 40 (19): c107985; and Sulea T. et al., Application of Assisted Design of Antibody and Protein Therapeutics (ADAPT) improves efficacy of a Clostridium difficile toxin A single-domain antibody. Sci Rep. 2018 Feb. 2; 8 (1): 2260).

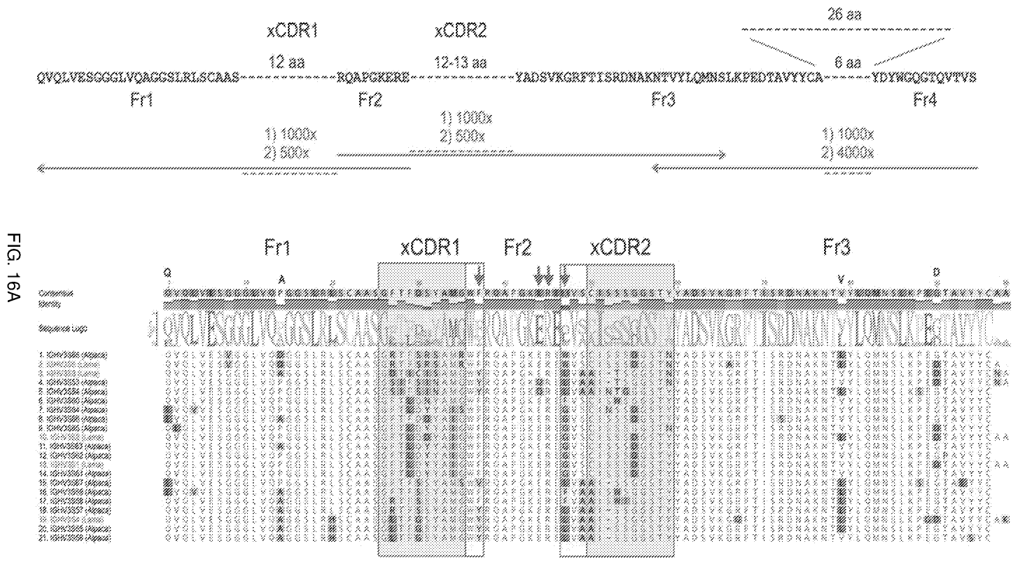

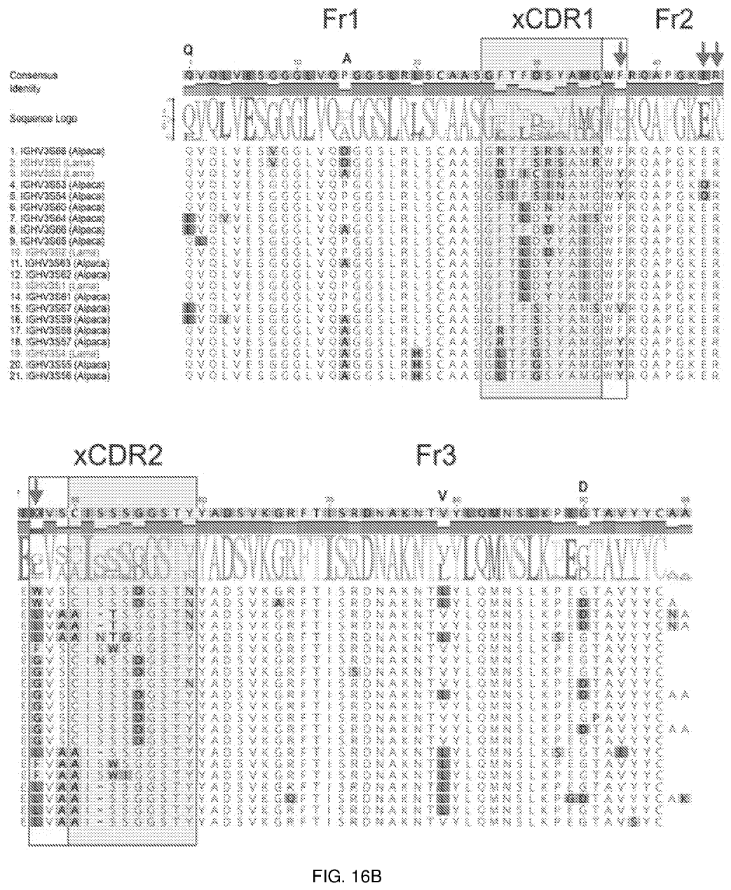

In the context of the present disclosure, the respective sequences of the Vin nanobodies is secondary to the point that, as would be recognized by a person of ordinary skill in the art, V_{HH} sequences and structures are very well-characterized and can be readily inserted into a yeast expression cassette, and in-frame with an FAP sequence in the fusion proteins described herein to achieve the desired antigen recognition for purposes herein. Though the framework region sequences, e.g., FR1, FR2. FR3, and FR4, are conserved, there still remains a degree of variability in those regions not only inter-species, but within species. As an example, sequences of NB framework and CDR regions are depicted in FIGS. 16A and 16B, with consensus framework regions depicted in FIG. 16A, and framework and CDR regions, including variations in framework sequences between NBs of alpaca and llama origin being depicted in FIG. 16B., the framework portions of which show at least ˜90% sequence identity with the exemplary consensus sequence: QVQLVESGGGLVQPGGSLRLSCAAS (X)ₘRQAPGKERE(X)ₙYADSVKGRFTISRDNAKNTV YLQMNSLKPEGTAVYYCA(X)ₚYDYWGQGTQVTVS (SEQ ID NO: 23, as shown in FIG. 16A and SEQ ID NOS: 45-48) where n is 12, m is 12 or 13, and p ranges from 6 to 26, inclusive, and (X)ₘ, (X)ₙ, and (X)ₚ correspond to CRD1. CRD2, and CDR3, respectively of the camelid VIII.

NB framework regions may be synthetically-modified to change one or more amino acid residues to improve any quality of the NB, and efficacy of those modified NBs are readily screened in the assay described herein. Further, because the purpose of the present disclosure is to provide a robust method and reagents for determination of affinity of a NB to a target protein, description of the specific sequence of the NB, especially of the CDRs is not relevant, though certain exemplary NB sequences are provided herein as illustrative. Reference to Mitchell, L S, et al. Proteins. 2018; 86:697-706 and Mitchell L S, et al. Protein Eng Des Sel. 2018-7-1; 31 (7-8): 267-275, as well as the multitude of additional references and sequences of NBs and camelid V_{HH} regions that are publicly-available, can guide a person of ordinary skill in the selection and mutagenesis (directed or random) of NB-encoding nucleic acids.

A full description of molecular cloning methods useful for production of proteins, including nanobodies, in yeast, including plasmids, clone selection, evaluation and propagation, and mutagenesis is routine and well within the abilities of a person of ordinary skill in the art. Further, cloning services are broadly-available both through research institutions and businesses. Exemplary methods are described herein and in U.S. Provisional Patent Application No. 63/290,408, to which the present application claims priority. As an example. Golden Gate cloning: Golden Gate cloning technology relies on Type IIS restriction enzymes, first discovered in 1996. Type IIS restriction enzymes are unique from “traditional” restriction enzymes in that they cleave outside of their recognition sequence, creating 1 to 5 base flanking overhangs. Since these overhangs are not part of the recognition sequence, they can be customized to direct assembly of DNA fragments. This feature allows for precise, scarless cloning in the final desired construct. The cloning scheme is as follows: the gene of interest is designed with Type IIS sites (such as BsaI or BbsI), that are located on the outside of the cleavage site. As a result, these sites are eliminated by digestion/ligation and do not appear in the final construct. The destination vector contains sites with complementary overhangs that direct assembly of the final ligation product (See, e.g., Marillonnet, S., & Grützner, R. (2020). Synthetic DNA assembly using golden gate cloning and the hierarchical modular cloning pipeline. Current Protocols in Molecular Biology. 130, e115, also, see, Otto M, et al. Expansion of the Yeast Modular Cloning Toolkit for CRISPR-Based Applications, Genomic Integrations and Combinatorial Libraries. ACS Synth Biol. 2021-12-17; 10 (12): 3461-3474).

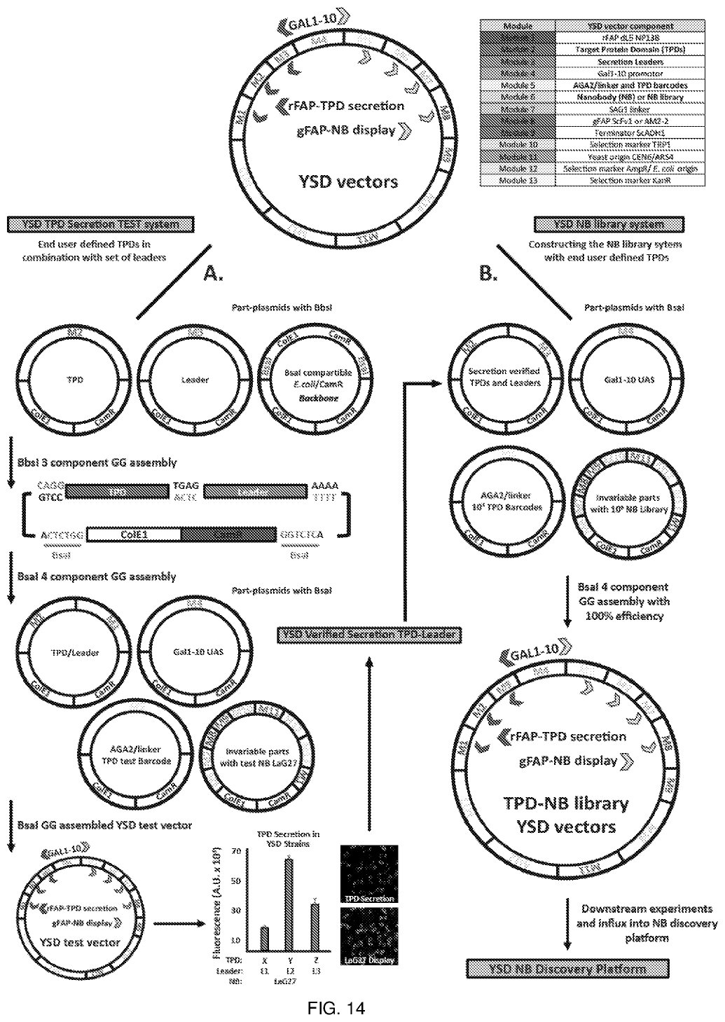

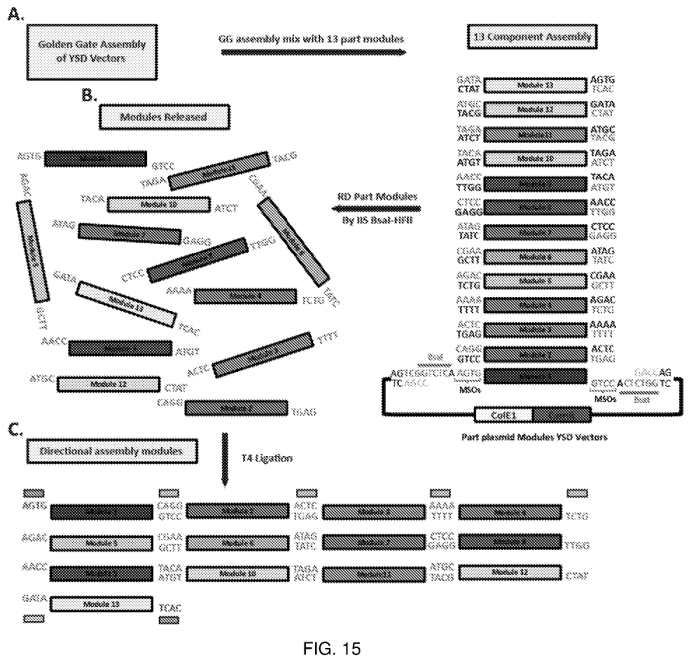

In the context of the present invention, a plasmid may be assembled using multiple type IIS restriction endonuclease-(type IIS RE-) digested DNA fragments, each comprising a useful gene, regulatory element, tag (e.g. barcode), or other useful sequence(s). Each fragment, which are referred to herein as modules, may include unique 5′ and 3′ overhangs after digestion for use in concatenating the fragments in order. Unique overhangs and endonuclease recognition sequences may be introduced at the ends of a desired functional sequence, such as a gene, by PCR using primers including both the recognition sequence of the type IIS RE and unique cutting sequences to yield unique nucleic acid modules for directional cloning. As shown below, such as strategy can be used to produce a highly-complex plasmid, with great efficiency. As shown in the examples below, a useful YSD plasmid may be produced using Golden Gate cloning methods using various type IIS RE recognition site-terminated DNA modules. In one example, the YSD nucleic acid is made using, in order, the following type IIS RE recognition site-terminated DNA modules: A first FAP ORF, a TPD ORF, a secretion leader ORF, a bidirectional promoter, a surface display leader ORF optionally including a TPD barcode, a NB ORF, an optional SAG1 linker ORF, and a second FAP ORF, wherein the first and second FAP bind different fluorophores that emit at different maximum emission wavelengths from each other and which cause an increase in fluorescent emissions from the bound fluorophores at their respective emission wavelength maximums. Additional useful modules include terminator sequences such as ScADH1, one or more selection markers such as TRP1 or Kan^{R}, a yeast replication origin, and/or an E. coli selection marker and replication origin so that the plasmid may be propagated in E. coli., all or may of which may be provided in a plasmid vector instead of a module for Golden Gate cloning. One of the first and second FAP ORF sequences may encode a malachite green-binding FAP such as a dL5 FAP, while the other of the first and second FAP ORF sequences may encode a TO1-binding FAP, such as a scFv1 or AM2-2 FAP. Modules may comprised more than one element, for example the first FAP ORF, TPD ORF, and secretion leader ORF may be provided as a single module (See, also, FIG. 14). Mutagenesis of the NB module may be performed in a separate plasmid, or in combination with other useful modules. Cloning of a library of diverse NBs may be performed by isolation of PBMC RNA from naïve or TPD- or antigen-immunized camelids and performing RT-PCR on the isolated RNA using PCR primers containing type IIS RE recognition sites to introduce unique type IIS RE recognition sites and overhangs for GG cloning purposes.

Libraries of VHH nanobodies may be synthetically engineered and optionally commercially synthetized or produced from nucleic acids of naïve or immunogen-stimulated, and optionally boosted camelids, such as alpacas of llamas. Variants may be generated by in vitro mutagenesis. Mutation of the nanobody, e.g., the amino acid sequences of the CDRs of the V_{HH} nanobody, likewise is well within the abilities of a person of ordinary skill, using, for example and without limitation, ethyl methanesulfonate (EMS) or ultraviolet (UV) light. Because the sequences of the V_{HH} nanobodies, and their variable and CDR regions are well-characterized (see, e.g., FIGS. 16A and 16B as examples), specific codons may be altered, e.g., within a CDR, and subsequently screened by the methods described herein, to obtain V_{HH} nanobodies with optimal affinity to an antigen. Sequence modification may be performed by any effective method, as are broadly-known to those of ordinary skill in the art.

Sequencing of clones, plasmids, barcodes, or DNA may be performed in any manner described herein, including next generation sequencing, also known as parallel or massively parallel sequencing (see, e.g., Alekseyev Y O, et al. A Next-Generation Sequencing Primer-How Does It Work and What Can It Do? Acad Pathol. 2018-5-6; 5:2374289518766521 and Behjati S, et al. What is next generation sequencing? Arch Dis Child Educ Pract Ed. 2013 December; 98 (6): 236-8). A barcode is a unique, clone-specific sequence that permits identification of a specific clone or class/group of clones. The DNA barcode may be placed in sequence within a fusion protein or in a cleaved region. For example, in the fusion proteins described herein, the barcode may be included in sequences encoding the stalk portion of the construct, or in a spacer (linker), such as a poly-glycinyl, serinyl, and/or alanyl spacer.

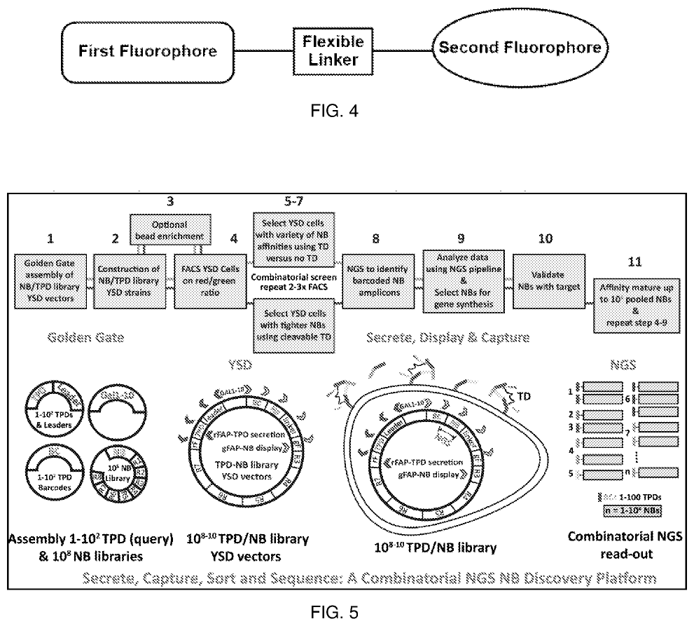

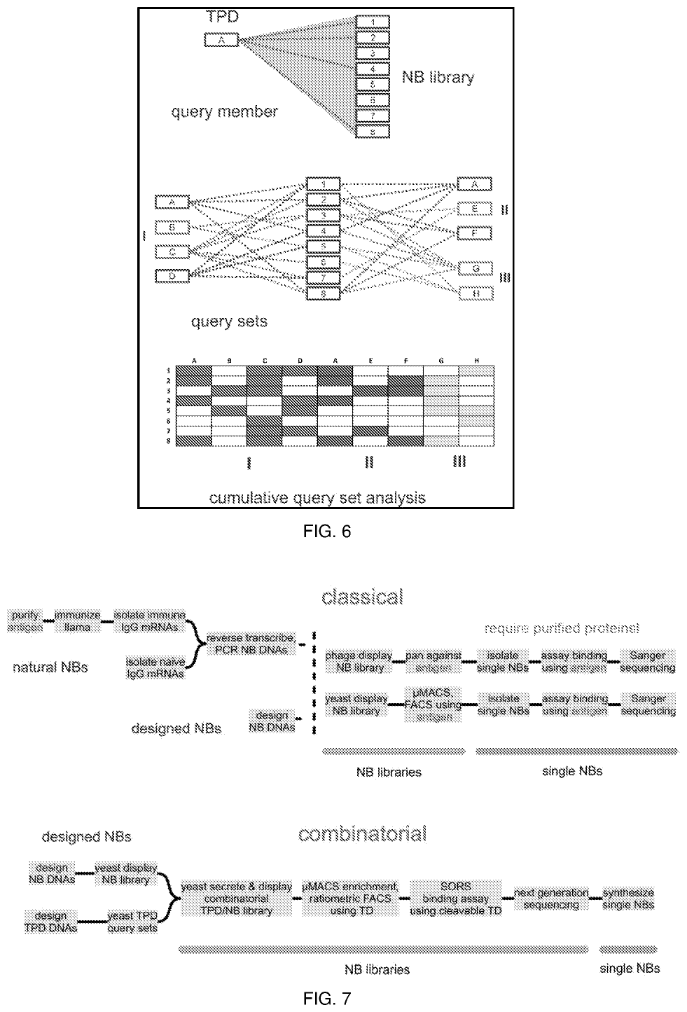

A tie-dye may be used to stabilize the complex of the first fusion protein and the second fusion protein. The tethered dye (“tie-dye”) is used in the tethered fluorogen assay (TEFLA) described herein. As used herein, a “tie-dye” or alternatively “tethered dyes”, is a compound that comprises a first fluorophore, such as MG, and a second fluorophore, such as sulfonated TO1, linked together or tethered together by a flexible linker (alternatively, spacer). Ackerman et al. demonstrate functionality of an exemplary tie-dye (Ackerman D S, et al., Bioconjug Chem. 2017 May 17; 28 (5): 1356-1362 used to label cell-cell contacts). An exemplary tie-dye schematic structure is shown in FIG. 4. The tie-dye may be used to quantitatively self-report the presence of the complex of the first fusion protein and the second fusion protein as a ratio of red to green fluorescence.

The flexible linker of the tie-dye is a moiety that is covalently attached to both the first fluorophore, such as MG, and the second fluorophore, such as a thiazole orange dye, such as the sulfonated TO1 dye depicted herein. Dye moieties may be linked to the linker or spacer moiety in any manner so long as the linkage does not interfere substantially with binding of the dye by an FAP. This can be readily tested, and further, malachite green dye and cyanine dyes can be linked as indicated below. The first fluorophore may be a triaryl methine dye such as a MG dye. The second fluorophore may be a monomethine cyanine dye. The flexible linker is between the first fluorophore and the second fluorophore. The flexible linker has no substantial negative effect on the activity of the first fluorophore or the second fluorophore, e.g., in context of the present invention, the ability of the first fluorophore, such as MG, to bind to the scFv (FAP) of the first fusion protein and the ability of the second fluorophore, such as sulfonated TO1, to bind to the scFv (FAP) of the second fusion protein.

The flexible linker may comprise a total length of from 1 nanometer (nm) to 50 nm, such as from 15 nm to 40 nm, such as from 20 nm to 35 nm, or such as from 25 nm to 30 nm. The linker may be long enough to sufficiently span the distance between the first fusion protein and the second fusion protein, e.g., between AM2-2 and dL5 in the NB/TPD complex. The flexible linker may comprise an O atom attached to a ring of an MG moiety forming an ether bond, one or more amide bonds, one or more methylene, dimethylene, or trimethylene moieties, and/or a triazole moiety. The flexible linker may also include one or more additional moieties, so long as those moieties do not interfere with the action or solubility of the respective fluorophore moieties.

The flexible linker may comprise one or more alkyl groups covalently bonded to one or more amide moieties, one or more triazole moieties, one or more ether moieties, and/or one or more poly(ethylene glycol) (PEG) moieties.

As used herein, “alkyl” refers to straight, branched chain, or cyclic hydrocarbon groups including from 1 to about 20 carbon atoms, for example and without limitation C₁₋₃, C₁₋₆, C₁₋₁₀ groups, for example and without limitation, straight, branched chain alkyl groups such as methyl, ethyl, propyl, butyl, pentyl, hexyl, heptyl, octyl, nonyl, decyl, undecyl, dodecyl, and the like.

As used herein, an “amide moiety” comprises the following structures:

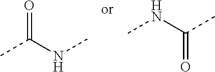

As used herein, an “ether moiety” comprises the following structure:

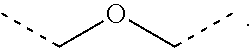

As used herein, a “triazole moiety” comprises the following structure:

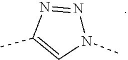

As used herein, a PEG moiety comprises the following structures:

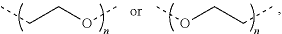

where n ranges from 1 to 200, e.g., from 1 to 100 (PEG₁₋₁₀₀), from 2 to 100 (PEG₂₋₁₀₀), or from 75 to 85 (PEG₇₅₋₈₅), such as 70 (PEG₇₀), 77 (PEG₇₇), 80 (PEG₈₀), 90 (PEG₉₀), or 100 (PEG₁₀₀).

The flexible linker may comprise the following structure:

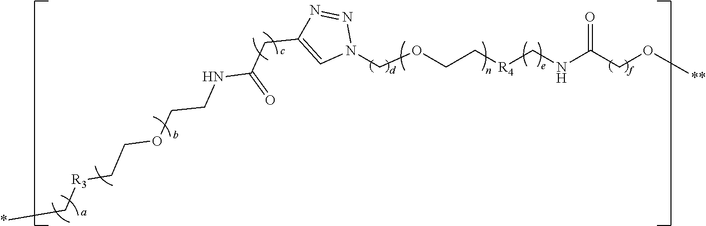

wherein a is an integer from 1 to 6, e.g., from 1 to 4, from 2 to 4, or 3; b is an integer from 1 to 200, e.g., from 1 to 100, from 2 to 50, or 2; c is an integer from 1 to 6, e.g., from 1 to 4, from 1 to 3, or 2; d is an integer from 1 to 6, e.g., from 1 to 4, from 1 to 3, or 2; n is an integer from 1 to 200, e.g., from 1 to 100, from, 2 to 100, from 75 to 85, or 80; e is an integer from 1 to 6, e.g., from 1 to 4, from 1 to 3, or 2; f is an integer from 1 to 6, e.g., from 1 to 4, from 2 to 4, or 3; R₃ and R₄ each independently are

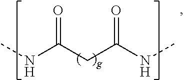

wherein g is an integer from 1 to 6, e.g., from 1 to 4, from 1 to 3, or 2; * indicates where the flexible linker binds to the second fluorophore; and ** indicates where the flexible linker binds to the first fluorophore.

The flexible linker may comprise the following structure:

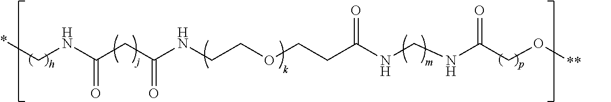

wherein h is an integer from 1 to 6, e.g., from 1 to 4, from 1 to 3, or 3; j is an integer from 1 to 6, e.g., from 1 to 4, from 1 to 3, or 2; k is an integer from 1 to 200, e.g., from 1 to 100, from 2 to 100, from 75 to 85, or 80; m is an integer from 1 to 6, e.g., from 1 to 4, from 1 to 3, or 2; p is an integer from 1 to 6, e.g., from 1 to 4, from 1 to 3, or 3; * indicates where the flexible linker binds to the second fluorophore; and ** indicates where the flexible linker binds to the first fluorophore.

The flexible linker may comprise the following structure:

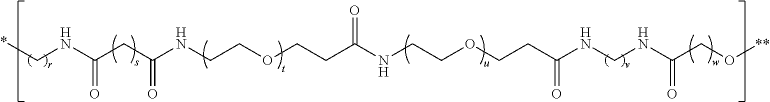

wherein r is an integer from 1 to 6, e.g., from 1 to 4, from 1 to 3, or 3; s is an integer from 1 to 6, e.g., from 1 to 4, from 1 to 3, or 3; t is an integer from 1 to 200, e.g., from 1 to 100, from 2 to 50, or 2; u is an integer from 1 to 200, e.g., from 1 to 100, from 2 to 100, from 75 to 85, or 80; v is an integer from 1 to 6, e.g., from 1 to 4, from 1 to 3, or 2; w is an integer from 1 to 6, e.g., from 1 to 4, from 1 to 3, or 3; * indicates where the flexible linker binds to the second fluorophore; and ** indicates where the flexible linker binds to the first fluorophore.

The flexible linker may comprise the following structure:

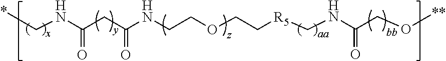

wherein x is an integer from 1 to 6, e.g., from 1 to 4, from 2 to 4, or 3; y is an integer from 1 to 6, e.g., from 1 to 4, from 1 to 3, or 3; z is an integer from 1 to 200, e.g., from 1 to 100, from 2 to 100, from 75 to 85, or 80; aa is an integer from 1 to 6, e.g., from 1 to 4, from 1 to 3, or 2; bb is an integer from 1 to 6, e.g., from 1 to 4, from 2 to 4, or 3; R₅ is

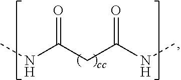

wherein cc is an integer from 1 to 6, e.g., from 1 to 4, from 1 to 3, or 2; * indicates where the flexible linker binds to the second fluorophore; and ** indicates where the flexible linker binds to the first fluorophore.

When the first fluorophore comprises MG, MG is covalently attached to the flexible linker as follows, optionally by an ether bond:

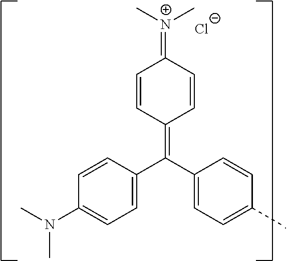

When the second fluorophore comprises sulfonate TO1, the sulfonate TO1 is covalently attached to the flexible linker as follows:

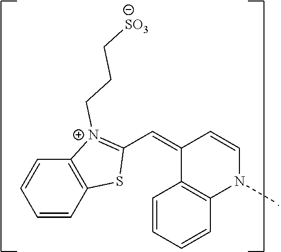

The tie-dye may comprise the following structure when the first fluorophore comprises MG and the second fluorophore comprises sulfonate TO1:

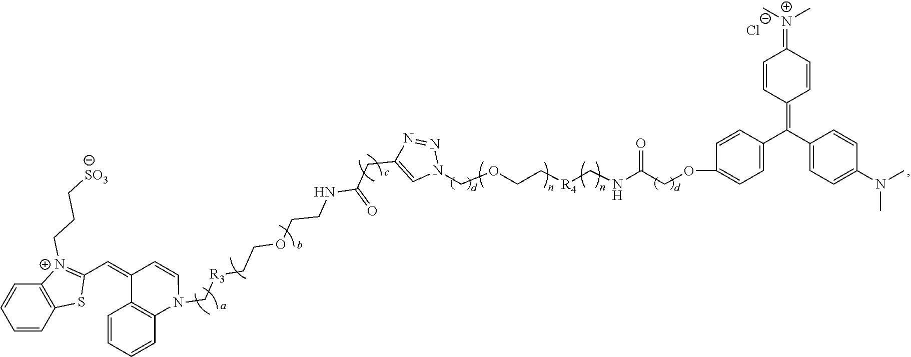

wherein a is an integer from 1 to 6, e.g., from 1 to 4, from 2 to 4, or 3; b is an integer from 1 to 200, e.g., from 1 to 100, from 2 to 50, or 2; c is an integer from 1 to 6, e.g., from 1 to 4, from 1 to 3, or 2; d is an integer from 1 to 6, e.g., from 1 to 4, from 1 to 3, or 2; n is an integer from 1 to 200, e.g., from 1 to 100, from 2 to 100, from 75 to 85, or 80; e is an integer from 1 to 6, e.g., from 1 to 4, from 1 to 3, or 2; f is an integer from 1 to 6, e.g., from 1 to 4, from 2 to 4, or 3; and R₃ and R₄ each independently are

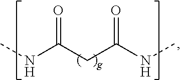

wherein g is an integer from 1 to 6, e.g., from 1 to 4, from 1 to 3, or 2. For example, the tie-dye may be sulfonate TO1-PEG₃₅₀₀-MG and may comprise the following structure:

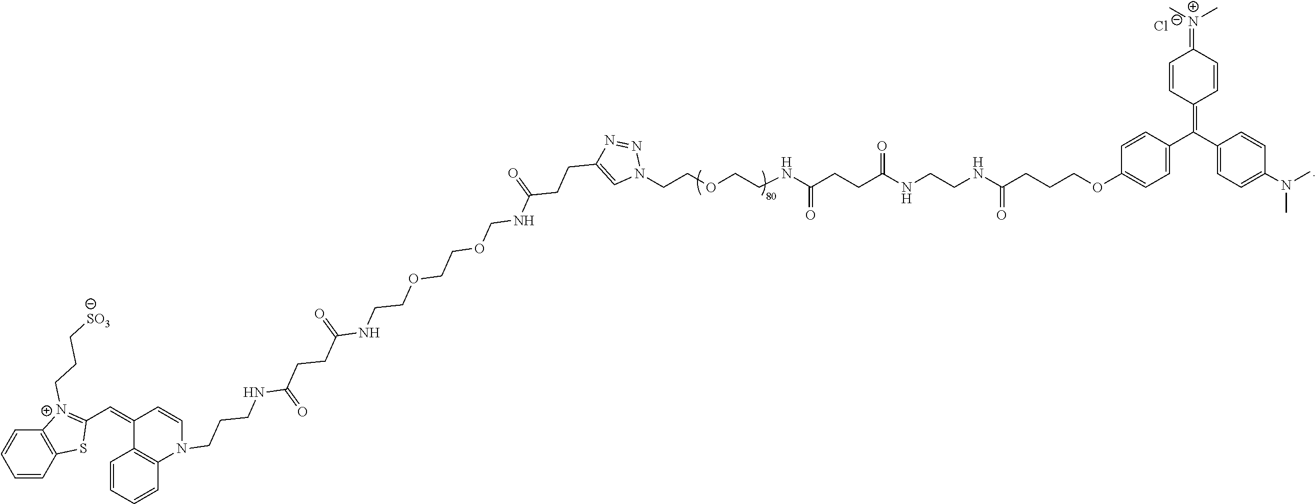

The tie-dye may comprise the following structure when the first fluorophore comprises MG and the second fluorophore comprises sulfonate TO1:

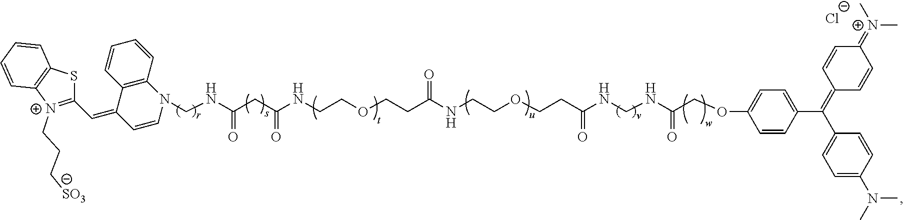

wherein r is an integer from 1 to 6, e.g., from 1 to 4, from 1 to 3, or 3; s is an integer from 1 to 6, e.g., from 1 to 4, from 1 to 3, or 3; t is an integer from 1 to 200, e.g., from 1 to 100, from 2 to 50, or 2; u is an integer from 1 to 200, e.g., from 1 to 100, from 2 to 100, from 75 to 85, or 80; v is an integer from 1 to 6, e.g., from 1 to 4, from 1 to 3, or 2; and w is an integer from 1 to 6, e.g., from 1 to 4, from 1 to 3, or 3.

The tie-dye may comprise the following structure when the first fluorophore comprises MG and the second fluorophore comprises sulfonate TO1:

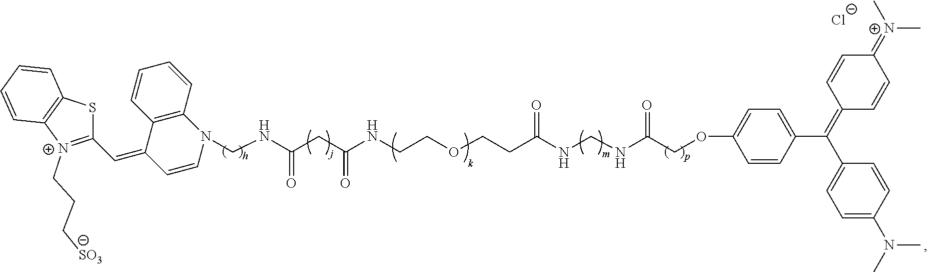

wherein h is an integer from 1 to 6, e.g., from 1 to 4, from 1 to 3, or 3; j is an integer from 1 to 6, e.g., from 1 to 4, from 1 to 3, or 2; k is an integer from 1 to 200, e.g., from 1 to 100, from 2 to 100, from 75 to 85, or 80; m is an integer from 1 to 6, e.g., from 1 to 4, from 1 to 3, or 2; and p is an integer from 1 to 6, e.g., from 1 to 4, from 1 to 3, or 3.

When the first fluorophore comprises MG and the second fluorophore comprises sulfonate TO1, the tie-dye may have the structure as provided in FIG. 11.

The following examples are provided as non-limiting illustrations of the subject matter described herein.

### EXAMPLES

Provided herein is a self-reporting single cell-based combinatorial NB screening technology, a major advance over current serial and parallel screening methods (See, FIG. 5). To support combinatorial NB screens, Golden Gate assembly is used to provide extremely flexible and efficient genetic engineering workflows required for mass fusion of the co-expression plasmids encoding both NB and TPD libraries. The YSD cell populations comprising NB/TPD libraries can be used in a combinatorial NB discovery platform to isolate and improve NBs against diverse, often hard to obtain, TPDs. We will then explore how true ‘many-on-many’ screening empowers the isolation of NBs able to distinguish between members of a set of related TPDs. Combinatorial screening using bioinformatics informed TPD query-sets will revolutionize the generation of multiple NBs that address difficult molecular recognition challenges in biology and medicine.

This NB discovery technology described herein represents a breakthrough in NB screening methodology and in how those immunoreagents will be used in future applications, as our system can be used in a ‘many-on-many’ NB screening mode, without physically purifying the NBs and Target Protein domains (TPDs). In a major advance, a model high-throughput yeast secrete and display (YSD) platform enables the isolation and identification, using next generation sequencing (NGS), of surface-displayed NBs that specifically bind secreted cognate TPDs. Fluorogen activating proteins (FAPs) are used in a dual fluorescence reporter system such that, in this proof-of-concept example, a secreted red FAP-TPD and displayed green FAP-NB cognate complexes generate intense red or green surface-specific fluorescence when exposed to cell-impermeant small molecule fluorogens. The TPD/NB complexes transiently formed on the yeast cell surface can be captured by a tie-dye (TD), where the fluorogens are linked by a flexible polymer linker. The TD stabilizes the NB/TPD complex and self-reports the complex in 2 colors on the yeast cells, which can then be sorted by 2-color ratiometric FACS. YSD cells bearing the cognate NB/TPD complexes are selected and the DNA encoding these complexes (NB sequences and TPD identification barcodes) are identified by NGS in a single read frame.

To support combinatorial NB screens, Golden Gate assembly is used to provide the extremely flexible and efficient genetic engineering workflows required for mass fusion of the co-expression plasmids encoding both NB and TPD libraries. The YSD cell populations comprising NB/TPD libraries is used to develop a combinatorial NB discovery platform to isolate and improve NBs against diverse, often hard to obtain, TPDs. “Many-on-many” screening empowers the isolation of NBs able to distinguish between members of a set of related TPDs. Combinatorial screening using bioinformatics informed TPD query-sets will revolutionize the generation of multiple NBs that address difficult molecular recognition challenges in biology and medicine.

As described below, our technology is based on fluorogen activating protein (FAP) technology. When FAPs are expressed as fusion reporters at the cell surface and exposed to cell-impermeant small molecule fluorogens (malachite green, MG; thiazole orange, TO1, in the following examples), intense red or green surface-specific fluorescence is respectively generated without the need for washes. For the yeast secrete and display platform, these fluorogens are linked by a flexible polyethylene glycol (PEG) linker to create a tie-dye (TD, or tethered dyes); if a NB specifically binds a TPD on the cell surface, the TD stabilizes the NB/TPD complex and self-reports the complex in 2 colors.

Simultaneously displaying a NB and secreting a TPD from the same cell is not trivial. Although many proteins of interest have been secreted from Saccharomyces cerevisiae with varied success using tailored strains and protocols, no-one has reported secretion of multiple proteins in a library context. For example, simultaneous display and secretion in our EBY100 strain will stress the cells and lead to high levels of cell death. After considerable trial and error, this major issue was solved using a vector selection marker that supports both display and secretion without stressing the cells. We have found it advisable to implement a variety of secretion leaders as different TPDs can be dependent on the leader for proper secretion (see, e.g., FIG. 13B). An unexpected behavior observed for most of the secreted TPDs is that they may initially be retained in the periplasmic space, and then released slowly to the cell surface, whereupon they can be stabilized by the TD in the presence of a cognate NB. Unlike others, we can monitor this behavior because we have small MW fluorogens that can access the periplasmic space, whereas high MW PEGylated fluorogens access only the outer cell surface. By this, fluorogen reagents can be tailored to detect only NB/TPD complexes on the outer cell surface.

Golden Gate (GG) assembly is helpful for building these YSD vectors because the system is modular and enables highly flexible design of scarless constructs. YSD reagents can easily be distributed in kit form to end-users, who can further adapt the YSD system to their own research needs. Of special importance going forward is the extreme efficiency of GG assembly, where thermo-cycled cutting/ligation reactions are driven to completion. This enables reproducible creation of high complexity NB libraries for each member of a TPD query set.

Nonimmune scaffold screens currently use purified target protein that are physically cloned and individually evaluated to isolate candidate binders. That resource-intensive approach is mitigated by the following: (1) A single cell YSD FACS FAP reporter system that reports the interaction of co-expressed NB and TPDs quantitatively in terms of specificity, affinity and kinetics, hence avoiding the use of purified protein; (2) A GG assembly platform that physically combines the encoded genotypes of co-expressed NB and TPD, enabling NGS analysis of FACS output to resolve complex yeast populations into the binding phenotypes of individual NB/TPD clones, therefore eliminating the need for physical cloning; (3) High throughput NB library screens that pool unrelated-often difficult to obtain-TPDs to simultaneously isolate multiple NBs, thus replacing current expensive and resource-intensive methodologies; (4) Combinatorial screens using bioinformatics derived TPD query sets to directly obtain groups of related NBs, thus avoiding serial evaluation of individual clones and creating a family of reagents that together address complex biological issues. These methods can be integrated into a simple and inexpensive high-throughput yeast NB discovery platform that can be distributed as NB toolkits with a wide variety of applications.

The green emitting fluorogen activating protein (gFAP) is genetically fused using GG assembly 3′ to each NB (NB-gFAP) where the gFAP may be ScFvl or AM2-2; the red emitting FAP (rFAP) is fused 3′ to each TPD (TPD-rFAP) where the rFAP is dL5. These ˜25 kD protein FAPs developed in our lab bind and activate cell impermeant fluorogenic dyes, eliciting cell surface-specific fluorescence with essentially no cross-reactivity. The NB-gFAPs are displayed on the cell surface as 3′ genetic fusions to AGA2p, a native yeast agglutinin, whereas the TPD-rFAPs are secreted. If co-expressed NB and TPD specifically bind one another, the TPD-rFAP will be captured on the surface of the co-expressing cell. However, its residence will be finite as determined by the binding affinity of the NB/TPD complex; most of the TPD-rFAP will be lost into the medium over the 3 day period of cell culture. To solve this issue, we linked the two fluorogens together via a flexible PEG linker (the TD) that efficiently captures/locks the displayed NB/TPD complex such that the TPD association with the surface is specifically and dramatically stabilized. This enables quantitative self-reporting of the presence of the NB/TPD complex as a ratio of red to green fluorescence. FACS will then be used to isolate cells displaying the complex. This simple assay eliminates the need for purified target proteins, a noteworthy step forward.

Initial FACS output populations expressing the TD specifically stabilize NB/TPD complexes over a wide range of NB/TPD binding affinities (tested K_{D}=0.18-22.600 nm). To identify tighter complexes, we propose to develop TDs with a photocleavable or a cleavable disulfide linker that can be broken by a light source or a reducing agent, respectively. In the initial grant, we extensively test-ed several strategies for the cleavable TDs based on duplex DNA; however, these TDs did not efficiently bind TPDs to the cell surface. As alternative strategies, we propose; (1) Bleaching the TO1 fluorogen that releases the fluorogen from the gFAP; (2) Dissociation of the TD by competitive replacement of TO1 from the NB-gFAP by using chemical agents (ML342); (3) Serial FACS cell sorting of populations expressing the non-cleavable TD stabilized NB/TPD complexes, followed by regrowth and a subsequent MG-10 KPEG plus TO1-10 KPEG assay to monitor the TPD dissociation of medium to higher affinity binders. When one of these strategies is efficiently operational, we can regrow the initial FACS output yeast population in the presence of cleavable or non-cleavable TD, and initiate synchronized TD cleavage or dissociation, which allows us to monitor TPD dissociation as a simple time course using FACS and NGS analysis. As used herein “SORS” refers to Synchronized Off-Rate Assay. 1st SORS yields values that are a good proxy for protein/protein binding affinity because off-rates are the major kinetic determinant of K_{D}. Also, 1st SORS can subsequently be used for the simultaneous affinity maturation of a relatively simple population of distinct NB/TPD complexes (10-100) by mutagenesis of the NB coding sequences within the GG assembled vectors; this capability is important because NBs initially isolated from non-immune libraries may have low binding affinities.

NB libraries of confirmed complexity can be independently constructed by Twist Bioscience (South San Francisco, California) and incorporated into our NB-library host plasmid (part-vector 4, invariable parts with 10⁹ NB library, FIG. 14). NB libraries are then fused with secretion-verified TPDs by employing a highly efficient 4-component GG YSD vector assembly platform. The assembled NB/TPD libraries can be transformed utilizing high efficiency yeast transformation methods to generate YSD libraries of single cells that co-express NBs and TPDs. The NB library complexity of the YSD strains can be tested and verified by NGS MiSeq platform data (˜5×10⁶ reads) using an empirical Bayes computational method. NGS reads can be processed by our custom designed NGS software pipeline based on open source software, where the critical analyses require efficient protein clustering.

Screening of NB libraries against highly multiplexed or combinatorial TPD sets requires a practical high throughput NGS strategy. Upon cell sorting and isolation, we observe a pool of red-green TD labeled cells representing as many as several thousand different NB/TPD complexes. NGS of the GG assembled vectors, containing the entire NB sequence and a specific barcode for the TPD within the same sequencing read, enables one to identify bonafide NB/TPD complexes within a population of cells because multiple instances of their sequences will be present. Indeed, MiSeq NGS and data analysis employing model NB/TPD screens reveals that the TPD sequences are accurately identified by the included TPD barcodes. This clearly demonstrates that the described YSD vector-tailored NGS strategy functions very efficiently (Example 1, experimental data). Using the obtained YSD NGS sequence information, one simply synthesizes the small protein coding regions of interest in the format needed rather than physically isolate individual clones encoding NB/TPD pairs. Nowadays, gene synthesis is routine and inexpensive, and re-introduction into yeast for further evaluation is made easy by GG technology or homologous recombination. This project constitutes the first functional integration of co-expressed probe and target proteins with NGS analysis based on directly encoded selectivity information (rFAP/gFAP). Successful integration with high throughput combinatorial screening would be a break-through in technology for the isolation and directed evolution of small single domain affinity reagents.

This YSD NGS platform is the first NB isolation technology capable of supporting truc combinatorial screening. The significant distinction between combinatorial and (massively) parallel NB/TPD screens is that every NB/TPD interaction is directly cross-referenced to every other NB/TPD in the combinatorial TPD query set library. Combinatorial screen data can be represented as an interaction matrix, either as plus/minus in a TD screen (FIG. 7), or more quantitatively in a SORS NGS ratiometric readout as changes in NB/TPD occupancy. The practical consequence is that one can directly compare in a single screen all binding properties without follow-up experiments. Parallel screens are more resource intensive, and unless there is some means to ensure that each TPD screening implementation and each NB library is identical, it is difficult to confidently cross-compare the aggregated data.

Combinatorial YSD screening is made possible by the extreme efficiency and fidelity of GG mediated ligation of each TPD into what is effectively, a single high quality standardized NB library. To ensure that nearly every NB in the library is represented in a TPD query, excess TPD can be ligated into an oversampled NB library. These libraries are successively transformed into E. coli and yeast using high transformation protocols to preserve library sequence complexity. Optimization and quality control of these steps establishes a rapid and reproducible work-flow for the YSD NGS platform. The NB/TPD combinatorial system is additive. Individual TPD queries can be easily combined to create configurable TPD query sets (FIG. 7). Different TPD query sets can be independently screened, and the datasets analyzed in a cumulative manner (FIG. 7). Combinatorial screens using TPD query sets lend themselves to complex applications requiring many NBs capable of distinguishing between query set members (FIG. 7). Because TPD query sets are relatively small (<100), computational methods may be required to extract useful query sets from big bioinformatics datasets.

Bioinformatics may be utilized to design TPDs based on domain structure, splicing junctions, point mutations, yeast codon optimization, and many other biological considerations. At no point is there a need to purify and characterize protein. The yeast secretory quality control apparatus is relied upon to ensure that the displayed NBs and secreted TPDs are expressed and properly folded. NB and TPD 3′ FAP tags serve as fluorescent quality control proxies; those FAP tags also function as direct fluorescent reporters for screens.

Advantages of this platform include: combinatorial multiplexed screening, it leverages genomic and structural bioinformatics, engineered TPDs support diverse screens, it is easy to pre-validate TPDs for secretion, NB and TPD libraries can be reused and reconfigured, NBs and TPDs are subject to eukaryotic quality control mechanisms, NBs bind self-recognized antigens, NBs may be rapidly isolated, there is no purification needed for rare or difficult antigenic proteins, no animal hosts are needed, expensive antibody reagents are not needed, this can be distributed as an relatively inexpensive kit (Sec, FIG. 7).

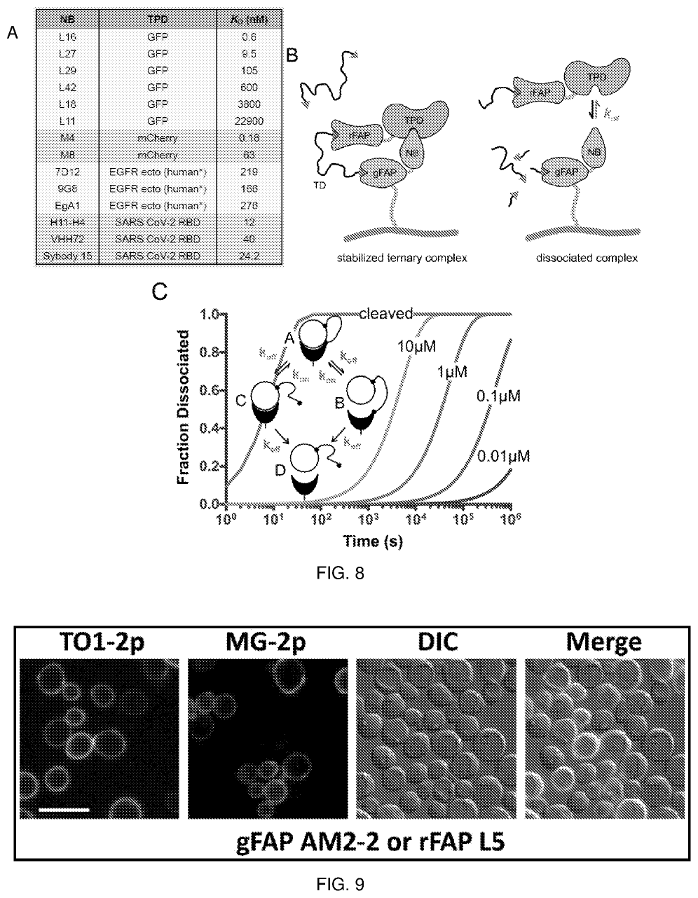

One approach is to directly build on the proven capabilities of our model YSD system (FIG. 8). NB/TPD GG vector kits can be produced that combine 2 high quality synthetic NB libraries with selected ectodomain TPDs, for example as shown below for-EGFR, SARS-CoV-2 spike RBD, and α-neurexin. These vector kits can then be used TDs to screen for novel cognate NBs using the NGS pipeline described below. High MW PEGylated fluorogens that broaden the screening capabilities of the YSD system are in development, including a branched TD to support inexpensive and rapid screens using magnetic beads, cleavable or dissociable TDs that support SORS for NB affinity characterization and maturation (1st SORS), and monovalent fluorogens that enable secondary screens for high affinity NBs (2nd SORS). These new fluorogen reagents can be initially tested using our existing panel of NB/GFPs, but then may be applied to screens for novel NBs. Small scale TPD query sets can initially be constructed to screen our 2 NB libraries. Bioinformatics can be applied to design TPDs based on domain structure, splicing junctions, point mutations, yeast codon optimization, and many other biological considerations. At no point is there a need to purify and characterize protein. The yeast secretory quality control apparatus is relied upon to ensure that the displayed NBs and secreted TPDs are expressed and properly folded. NB and TPD 3′ FAP tags serve as fluorescent quality control proxies; crucially, these FAP tags also function as direct fluorescent reporters for screens.

### Example 1—Construct and Test Modular NB/TPD Library Vector Kits for NGS-Based Identification of NBs Able to Combinatorially Discriminate Among Multiple User-Defined TPDs

Capture methods and model Golden Gate expression constructs have been developed that enable cell surface displayed NBs to specifically capture co-expressed secreted TPDs, and the NB/TPD complexes can be identified by NGS. Efficient expression and secretion of TPDs may be highly case-specific, necessitating flexible vector designs that enable the user to design and validate TPDs prior to inclusion in the NB/TPD library.

A one-pot Golden Gate system was built, based on proven secrete and display vectors, that quantitatively assembles a synthetic NB library with 3 TPD components—a TPD ID barcode, a secretion leader, and the TPD coding sequence. These vector kits are tested by building 2 different NB libraries that are screened for novel NBs that bind several functionally and structurally varied cctodomain TPDs including human EGFR (epidermal growth factor receptor). SARS-CoV-2 spike protein, and mouse α-neurexin.

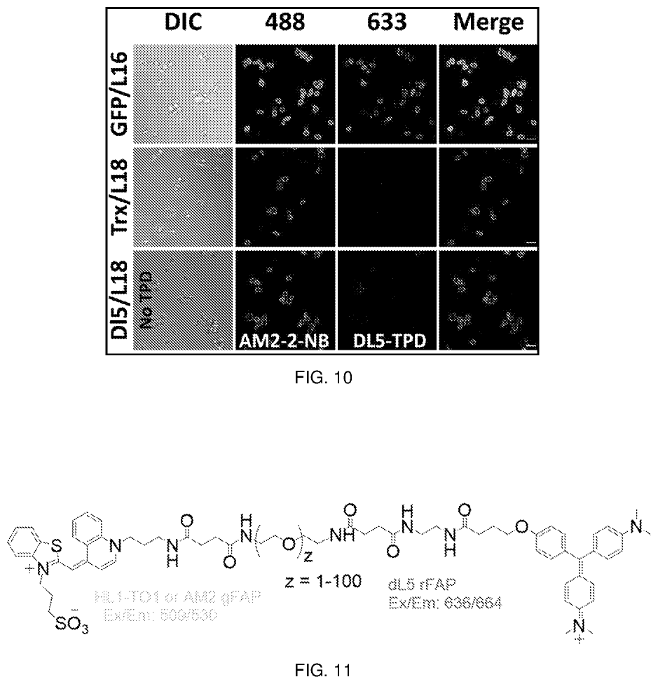

The YSD platform is empowered by FAP technology and can be implemented using well-established high speed FACS methods to isolate and affinity mature small affinity reagents. Using FAPs to label cell surface epitopes has major advantages in large library FACS screens over more traditional yeast display FACS screens that still rely on antibodies and often secondary antibodies, which are very expensive and inconvenient due to binding and dissociation artifacts, and required washing steps. FAPs alleviate all these deficiencies; one simply adds fluorogen to the library to attain saturated equilibrium binding to achieve stable high-level fluorescence with little to no background signal (FIG. 9). By employing two different FAPs, one can implement efficient quantitative ratiometric FACS screens that are powerful, precise, and inexpensive. In the YSD strains we use dual FAP reporters fused to secreted TPDs and displayed NBs that interact on the yeast cell surface (FIG. 10). This self-reporting NB/TPD capture system is the basis of ratiometric technologies that monitor intrinsic proper-ties of the NB/TPD complexes of interest.

### Example 2—Development of New PEGylated Fluorogens to Improve Screening, Characterization and Evolution of NBs

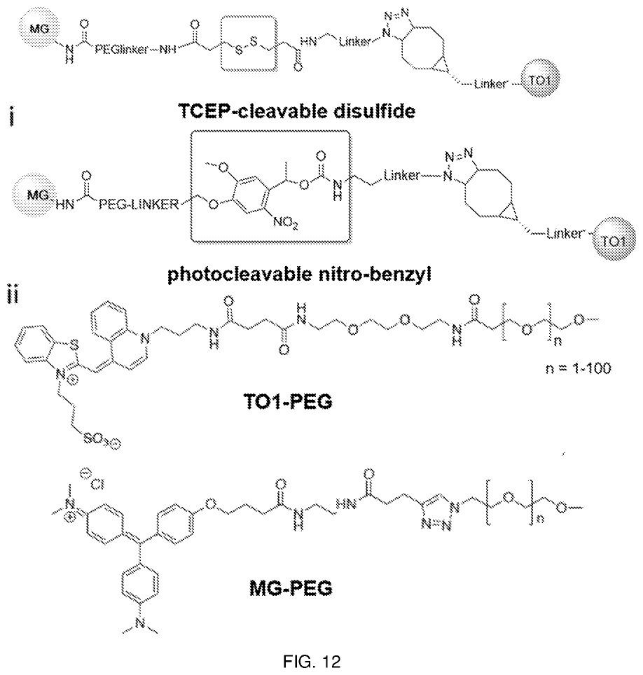

Our imaging center has developed many tandem dye reagents. Environmentally sensitive FRET donor dyes anchored to MG act as sensors of in vivo environments when bound to dL5 FAP; branched and polymeric dye reagents that are PEG tethered to MG act as FRET signal amplifiers when bound to dL5 FAP. In recent years, we synthesized PEG-linked TDs that interact with two distinct FAPS, for the purpose of monitoring the proximity of proteins fused to green and red FAPs, respectively. The TO1-PEG-MG tandems may be preferred as the spectral properties and robustness of these fluorogens bound to HL1-TO1. AM2 and dL5 FAPs are ideal for excitation by ubiquitous 488 nm and 633 nm lasers (FIG. 11). The length of the flexible PEG linker (10-100 monomers) should be longer than the expected FAP separation distance; additional junctions and cleavable chemistries can incorporate within the PEG linker. Self-reporting TD fluorogens are unusually amenable to such validation. For the YSD strains, the TO1-PEG₃₅₀₀-MG (˜77-mer) TD version was employed and validated its capacity to lock/capture the NB/TPD complexes. This TD is highly specific and efficiently locks (Kp's ranging up to ˜ 20 μM) the NB-TPD complexes on the yeast cell surface. This behavior illustrates that the TO1-PEG₇₇-MG TD is functional as a bivalent two color proximity reporter and can be efficiently used for red/green ratioing of the TPD-rFAP/NB-gFAP complexes. As such, copper free click chemistry may be used to synthesize two classes of cleavable tie-dyes (based on TO1-PEG₇₇-MG) that will be utilized for quantitative red/green ratiometric assays with 1st SORS. High MW PEGylated fluorogens may be used as an alternative strategy for ratioing (FIG. 12): (i) A TCEP cleavable disulfide S-S TD and a nitro-benzyl photocleavable TD (blue box); (ii) As a series of PEGylated TO1 and MG fluorogens as alternative (2nd SORS) method for SORS. We have extensive experience in generating these types of TDs and high MW PEGylated fluorogens.

PEG-linked fluorogens (‘tie-dyes’. TDs) specifically stabilize and capture on the yeast surface NB/TPD complexes with binding affinities ranging up to ˜20 μM. Secreted TPDs are initially sequestered in the yeast periplasmic space and/or inner cell wall; upon buffer treatment TPDs are slowly released to the cell surface where they can be selectively bound and captured by surface-restricted PEGylated fluorogens, including TDs. Three new types of PEGylated fluorogens are in development: (i) a branched TD variant that adds a biotin affinity label to support efficient magnetic bead enrichment of large libraries for NB/TPD binders; (ii) Green and red monovalent fluorogens bearing high MW PEG to support secondary ratiometric FACS screens (2nd SORS) that identify high affinity NB/TPD complexes within TD-enriched populations; and (iii) cleavable or dissociable TD variants for an NGS-based synchronized offrate screen (1st SORS) as a proxy for a bulk NB/TPD binding affinity assay. 1st SORS may be used for bulk affinity maturation of 10-100 selected NBs that recognize 10-100 distinct TPDs.

### Example 3—Use of Different Types of TPD Query Sets to Explore Combinatorial Screens for NBs that Report on Specified Biological Attributes

As end-user ready vector kits, a GG system is built and tested, based on model YSD vectors, that al-lows easy validation of TPD secretion and subsequently quantitative assembly of the TPD-NB libraries. Novel NBs are screened against several functionally and structurally distinct high value TPDs.

NGS screening using user-defined sets of TPDs enables one to devise novel combinatorial screening strategies. A query set (or pooled small sets) of up to ˜100 total TPDs may be designed to build focused thematic toolkits by comparison of NBs that differentially bind its members. Based on prototypical TPDs validated as described above. 3 distinct types of small query sets can be developed that address: (i) epitopes on GFP mapped by alanine scanning; (ii) mutations (variants) of the SARS-CoV-2 spike protein; and (iii) splicing isoforms of mouse α-neurexin.

To expedite the technological development a well-established 13-piece model GG assembled YSD vectors is produced as the basis (FIGS. 13 and 14) to create a redesigned GG system compatible with the combinatorial NB discovery technology.

A 4-piece GG system is first built that enables; (1) convenient testing of TPD secretion leaders to ensure proper TPD secretion in yeast before the TPD/NB libraries are combined; (2) driving GG assembly to completion so that incomplete fusion of the GG-assembled YSD TPD/NB vectors will not lead to loss of NB library complexity; (3) location of the barcode close to the NB sequence to ensure maximal coverage in NGS NB paired-end reads, to limit errors in NGS reads, and to in-crease our flexibility in NB library design. The end-user vector kit will be comprised of parts A and B; (A) the end-user defined TPD secretion system and (B) the TPD/NB library fusion system (FIG. 14). FIG. 14 depicts an exemplary GG cloning process involving Modules 1-13. Modules are excised from part plasmids, e.g. as depicted in FIGS. 13 and 14, by Type IIS restriction digestion. Released modules are assembled into YSD vectors by multiple ligation cycles. Proper assembly is easily assessed by restriction digestion. 98% of assessed constructs were correctly assembled.

Two different large complexity (10⁹) NB libraries are built making use of the functional germline sequences of camelids while availing synthetic technology that maximizes sequence complexity and sequence space coverage and minimizes sequence errors. The NB framework region Fr1 to Fr3 is derived from consensus sequences and Fr4 is created by V (D) J recombination that allow us to simply chose the J sequences from current NB libraries. Consensus scaffolds can be tested using pre-validated CDR swap-ins. This enables construction of NB libraries based entirely on CDR variation using precise biologically functional amino acid sequences culled from known CDRs (scc, e.g., FIGS. 16A and 16B). Subsequently, the functionality of this NB discovery technology can be tested by screening for new NBs against several different TPDs previously validated in model system (Mcherry, GFP. EGFR and SARS-CoV-2). We also plan to screen for bacterial surface proteins of Streptococcus pneumoniae. This will be followed by screening for novel NBs against the challenging target neurexin.

### Example 4—Synthesis of Sulfonate TO1-PEG₃₅₀₀-MG

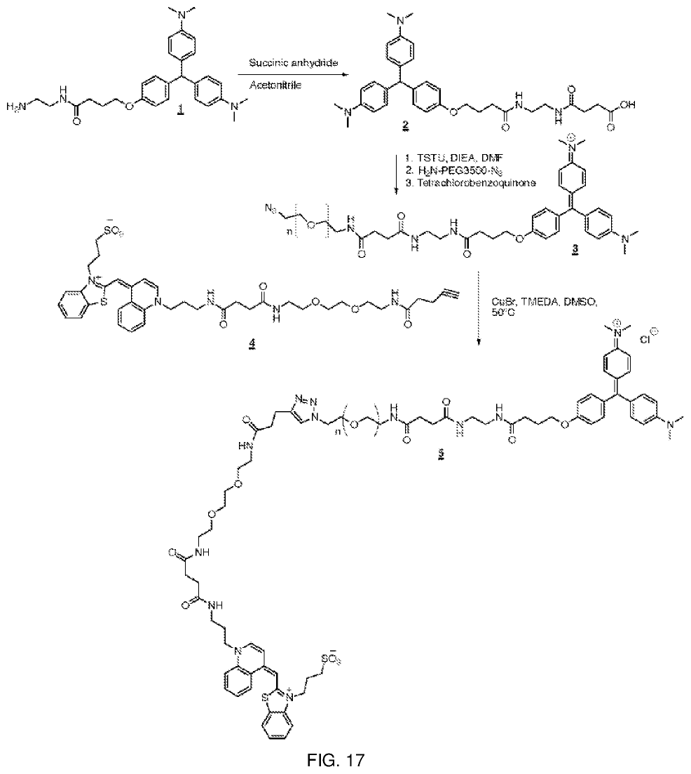

The MG-PEG₃₅₀₀-TO1 tie-dye was prepared according to the synthesis scheme depicted in FIG. 17. Briefly, N-(2-aminoethyl)-4-(4-(bis(4-(dimethylamino)phenyl)methyl) phenoxy)-butanamide (Compound 1) (474 milligrams (mg), 1 millmole (mmol)) was dissolved in anhydrous acetonitrile (5 milliliters (mL)). Succinic anhydride (100 mg, 1 mmol) dissolved in anhydrous acetonitrile was added dropwise to Compound 1 under stirring. Stirring was continued at room temperature overnight. The solvent was removed to give a resinous solid of 4-((2-(4-(4-(bis (4 (dimethylamino)phenyl)methyl) phenoxy) butanamido)ethyl)amino)-4-oxobutanoic acid (Compound 2).

Compound 2 (100 mg, 0.17 mmol) was dissolved in dry dimethylformamide (DMF, 0.5 mL). N,N,N′,N′-Tetramethyl-O—(N-succinimidyl) uronium tetrafluoroborate (TSTU, 60 mg. 0.2 mmol) and N,N-Diisopropylethylamine (DIPEA, 0.035 mL, 20 mole (mol)) were added. The reaction mixture was stirred for 1 hour at 50 degrees Celsius (° C.). Amino-PEG₃₅₀₀-azide (350 mg. 0.1 mmol) was added, followed by DIEA (0.0175 mL, 10 mmol). The reaction mixture was stirred overnight at 50° C. The solvent was removed and the residue was taken up in 5 mL of acetonitrile. The reaction mixture was heated up to reflux and tetrachlorobenzophenone (50 mg. 0.2 mmol) was added. The reaction mixture was refluxed for one hour, cooled to room temperature, concentrated and purified by chromatography on silica gel (eluent: chloroform/methanol 0-20% to give 220 mg of Compound 3).

4-Pentynoic acid was suspended in dry chloroform. Oxalyl chloride was added and the reaction mixture was heated under reflux until the gas evolution stopped. The reaction mixture was cooled to room temperature and the solvent was removed under vacuum. The remaining oil was used as such in the next step. Benzoxazolium, 2-[[1-(17-amino-5,8-dioxo-12,15-dioxa-4,9-diazaheptadec-1-yl)-4 (1H)-quinolinylidenc]methyl]-3-(3-sulfopropyl)-, inner salt TO1-2p1 was dissolved in acetonitrile, 1 equivalent of DIEA was added and 4-Pentynoic acid chloride was added dropwise. The reaction mixture was stirred at room temperature overnight, concentrated and purified on silica gel (eluent: chloroform/methanol) to form (Z)-3-(2-((1-(5,8,19-trioxo-12,15-dioxa-4,9,18-triazatricos-22-yn-1-yl) quinolin-4 (1H)-ylidene)methyl)benzo[d]thiazol-3-ium-3-yl) propane-1-sulfonate (Compound 4, TO1-2p-alkyne)

Compound 4 (76.5 mg, 0.1 mmol) and Compound 3 (410 mg. 0.1 mmol) were dissolved in dimethylsulfoxide (DMSO, 3 mL). The solution was purged with argon for 10 minutes. Copper (I) bromide (14 mg. 0.1 mmol) was added, followed by tetramethylethylenediamine (TMEDA, 15 microliters (uL), 0.1 mol) and the reaction mixture was stirred at 50° C. for 2 days. The reaction mixture was precipitated by drop wise addition to ethyl acetate (20 mL). The organic phase was decanted and the residue taken up in water and purified by size exclusion chromatography on Bio-Gel P-2 using water: 20% acetonitrile as eluent to form sulfonate TO1-PEG₃₅₀₀-MG (Compound 5).

### Example 5

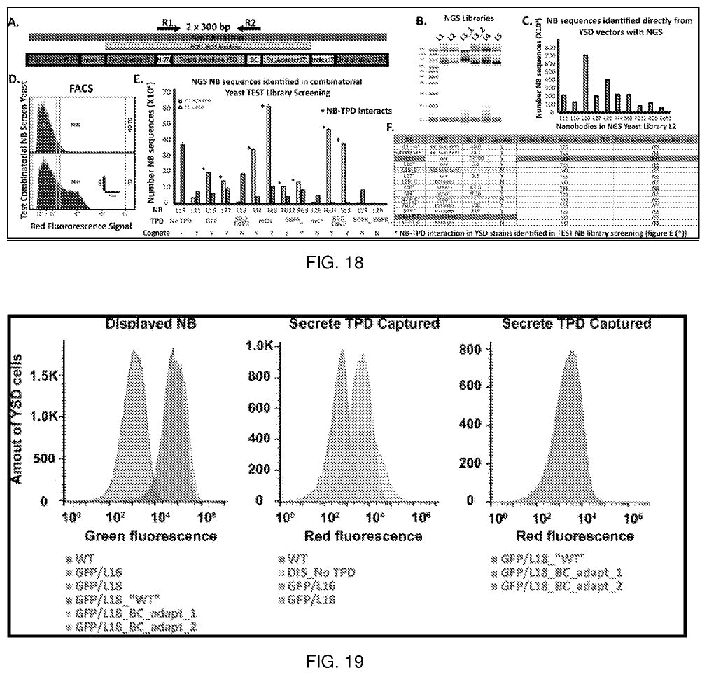

The model NB discovery technology was developed and validated. The following were demonstrated: (i) secretion of a variety of the TPDs; (ii) capture with TD of cognate TPD/NB pairs on the yeast cell surface; (iii) accurate sorting of cells expressing cognate NB/TPDs; and (iv) decoding the sequences of NBs present in cognate NB/TPD complexes (FIG. 18). In designing GG YSD assembly vectors, two different options for placement of the barcode were tested. The barcode falls within the linker between AGA2p and the NBs and can affect several parameters of our system (NB display, NB/TPD interactions, and NB/TPD capture efficiency). However, barcode replacements in the AGA2 linker showed no differences for these parameters, and is the same in comparison to the control (barcode not replaced). This strongly indicates that we can replace the barcode region without affecting NB display and cognate NB/TPD interactions in YSD strains. (FIG. 19).

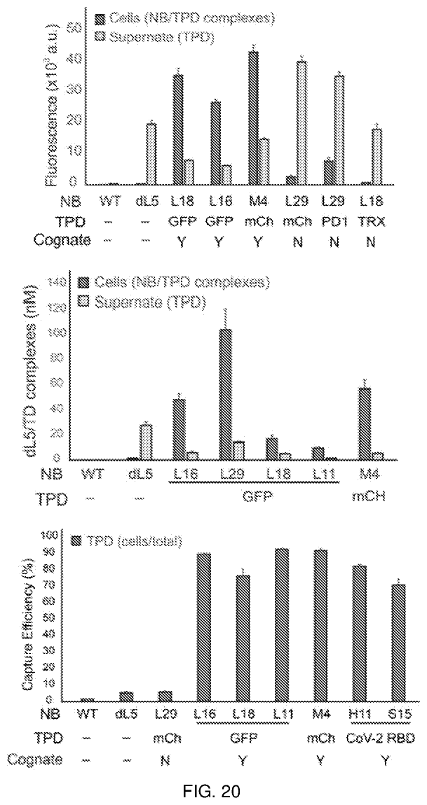

NB recognition & capture of secreted TPDs on the cell surface: model strains provide detailed information using a plate reader. Cells grown for 3 days with TD, harvested and divided into cell & supernatant fractions. All assays were carried out at 636/664 nm excit./emiss. to detect dL5/MG. Quantitation of dL5/MG employed calibration curve constructed from purified dL5 & MG-2p. As shown in FIG. 20, cognate NB/TPD complexes clearly retain their predicted recognition specificity and TPD-rFAPs are secreted & displayed at total levels of 10-100 nM. These variations highlight the need for robust red/green ratiometric FACS screening. Calculated efficiencies of capture of TPDs by cognate NBs range from 70-90%. Apparent efficiencies of capture of TPDs by non-cognate NBs are less than 10%.

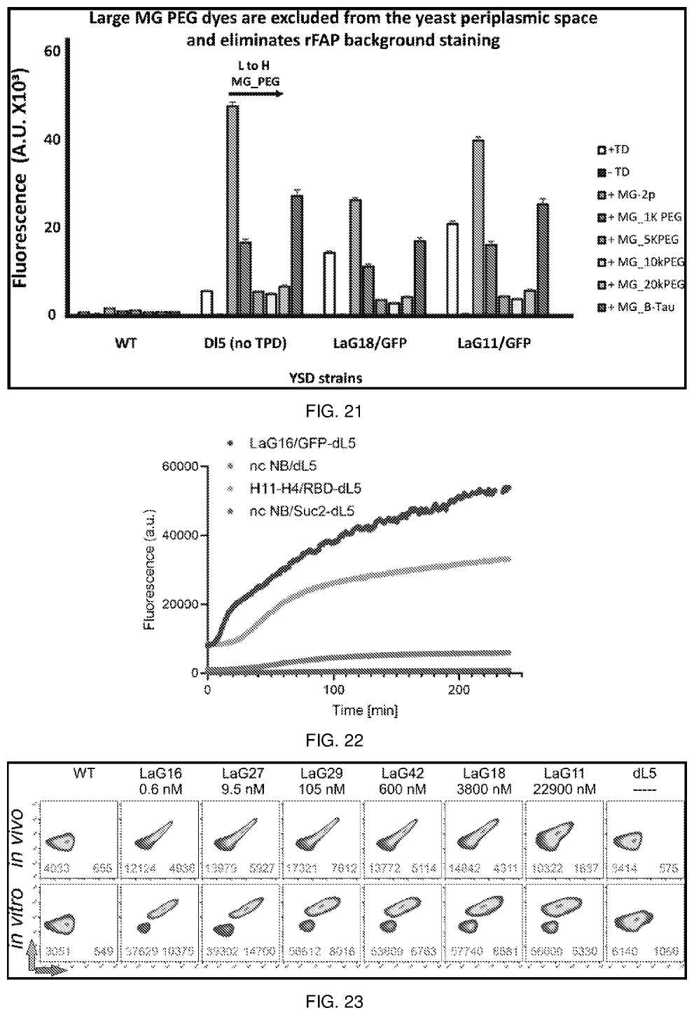

The well-validated YSD GG assembly vector platform is leveraged by dividing the 13-piece YSD vector into 4-pieces without changing YSD vector sequences. This creates flexibility in use and solves limitations in current YSD vector assembly that impede development of highly efficient combinatorial NB discovery platforms. Four modules are built: module 1, TPD and secretion leader; module 2, Gal1-10 UAS; module 3, barcodes; and module 4, the NB library with the remaining elements (see FIG. 13A; M7-M13 and M1) to create a fully functional YSD vector (see, also, FIG. 13B). By reducing the number of modules in the GG assembly process, GG assembly efficiency is increased significantly, ensuring that the 4-piece assembly of these NB/TPD library vectors progresses close to 100% completion, thus enabling assembly without losing NB library complexity. For part-plasmid module 4 (invariable parts with 10⁹ NB library), a customized IIS BbsI swap-out stuffer fragment cassette located at the NB site is used to create the NB basic construction vector. This allows easy incorporation of the designed NB libraries or, at will, individual fusion of any desired NB. The designed libraries may be constructed and validated for library complexity (10⁹) by Twist Bioscience and then cloned into the NB basic construction vector to create the NB library part-plasmids (component 4. FIG. 14). The NB complexity of the YSD strains is validated by NGS data (MiSeq platform. Illumina) and analyzed with our custom designed NGS pipeline. The complexity will be calculated from the NGS data (˜5×10⁶ reads) using an empirical Bayes computational method. Each constructed NB/TPD YSD library is screened for novel NBs against their targets (EGFR. SARS-CoV2 and α-Neurexin) by first employing the NB discovery platform using the non-cleavable TD. An optimization step that eliminates the rFAP/MG-2p TPD background signal due to retained TPDs in the yeast periplasm by using large PEGylated dyes that cannot penetrate into the periplasmic space (FIG. 21). When the cleavable TD is tested and validated, these constructed NB-TPD YSD libraries are re-screened for novel NBs against their targets using the cleavable TD system, the mature NB discovery platform is tested.

Three types of PEGylated fluorogens are in development: (i) a branched TD variant that adds a biotin affinity label to support efficient magnetic bead enrichment of large libraries for NB/TPD binders; (ii) Green and red monovalent fluorogens bearing high MW PEG to support secondary ratiometric FACS screens that identify high affinity NB/TPD complexes within TD-enriched populations; and (iii) cleavable or dissociable TD variants for an NGS-based synchronized off-rate screen (2nd SORS) for a bulk NB/TPD binding affinity assay and for NB affinity maturation.

The TD (TO1-PEG₇₇-MG) is shown to selectively bind and stabilize NB/TPD complexes on the yeast cell surface and restriction to the surface depends on PEGylation. Based on this paradigm ancillary PEGylated fluorogens are being built to support the NB discovery pipeline.

The secreted ectodomain TPD-rFAP fusions are initially sequestered within the yeast periplasmic space and/or inner cell wall, and upon buffer treatment released to outer cell surface over the course of a few hours (FIG. 22). TDs based on unmodified PEG polymers efficiently captured NB/TPD complexes on the outer cell wall. However, cleavable TDs for 1st SORS that included 18 bp of dsDNA plus a 6 bp ssDNA ‘toc-hold’ to mediate cleavage by strand-displacement behaved very differently; extensive studies demonstrated very rapid TD cleavage but inefficient association of the TD with the negatively charged yeast cell surface (data not shown). Those differences are attributed to the negatively charged and relatively stiff DNA duplex, in contrast to the flexible physicochemically inert PEG polymer. The salient feature of TDs is the efficient specific capture of TPDs by cognate NBs at affinities as low as 20 μM; this affinity range is far greater than for typical yeast displayed libraries, where NBs isolated after FACS enrichment have affinities <1 μM (FIG. 23). A second feature of TDs is that they can be successfully applied to cells post-harvest instead of the usual inclusion of TDs in growth medium for 3 days. Initial work indicates that post-harvest application expands the affinity range of efficient NB/TPD capture (FIG. 23) and markedly improves fluorogenic signal/noise, leading to better resolution on red/green ratiometric FACS screens (FIG. 23). More efficient ratiometric FACS screens will support more productive identification of cognate NB/TPD complexes using NGS. The functionality of other PEGylated fluorogens may be significantly improved if they can be used post-harvest because one can control buffer additives (ions, detergents, competitors) that modulate their binding.

TDs can potentially capture large combinatorial repertoires of NB/TPDs from synthetic NB libraries of moderate (10⁷-10⁸) complexity by several rounds of FACS. To make this enrichment methodology simpler and cheaper. TDs are adapted for use in magnetic bead enrichment of yeast surface displaying cells by synthesizing a branched variant that incorporates biotin. Each yeast cell displays as many as 100,000 NB/TPD complexes, and full occupancy of these complexes by biotinylated TD would make the cells prone to saturation with microbeads, driving up costs and increasing chances of non-specific retention of cells on the magnetic columns. This can be remedied by using defined mixtures of TD and biotinylated TD, which will provide fine control over binding to the μMACS beads.

For use in a follow-up screen of TD-captured NB/TPD repertoires, we will synthesize green and red fluorogens carrying 10 kDa PEG tails are synthesized, see FIG. 12 (ii). These surface-restricted reagents allow use of ratiometric FACS screening and NGS to semi-quantitatively assess which NB/TPD complexes are relatively tight. Because NB/TPD interactions are not stabilized by TD, the observed quantities of TPDs on the cell surface will reflect a dynamic time-dependent process that is primarily a balance between the time of appearance of TPDs on the surface (˜4 hrs) and subsequent dissociation from the NB/TPD complex (K_{off}). We can roughly estimate that complexes with Koff rates slower than 10⁻⁴ S-1 (FIG. 22) will still be present in significant quantities after 4 hours; this corresponds to a K_{D} of 0.1-1 nM given typical NB/antigen on-rates of 10⁵-10⁶ M⁻¹ S⁻¹. However, these PEGylated reagents can be applied at any time post-harvest to buffer-washed cells and immediately implement FACS, thereby constructing a time course that at early times better represents the presence of much looser NB/TPD complexes. This empirical approach will require testing and calibration using the full range of affinities of our model NB/GFP system. We already have evidence that straightforward labeling with 10 KDa PEGylated MG works well for detecting TPD/NBs with the whole range of K_{D}s of 0.2 nM to 22.600 nM even when the fluorogen is added to growth medium (data not shown), and we anticipate that using this approach post-harvest will be more robust. Because we will be analyzing by NGS a time course of relative changes that reflect countervailing processes in the abundance of a potentially large repertoire NB/TPD complexes, it is important that FACS be immediate and that a statistically robust unbiased population of cells be collected at each time point. For example, at T=0, 15, 30, 90 and 240 minutes, sort 10⁷ cells gated on all expressing cells at 50,000 cells/see (200 seconds).

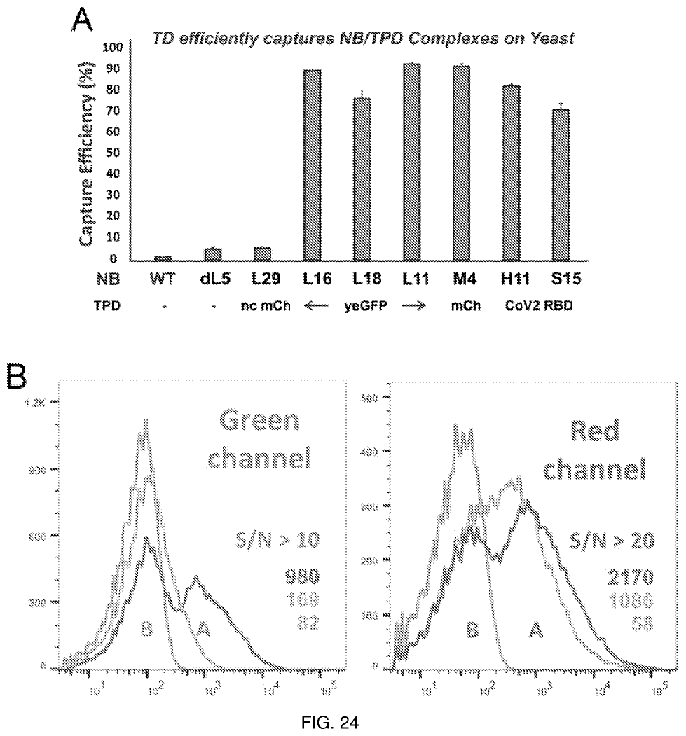

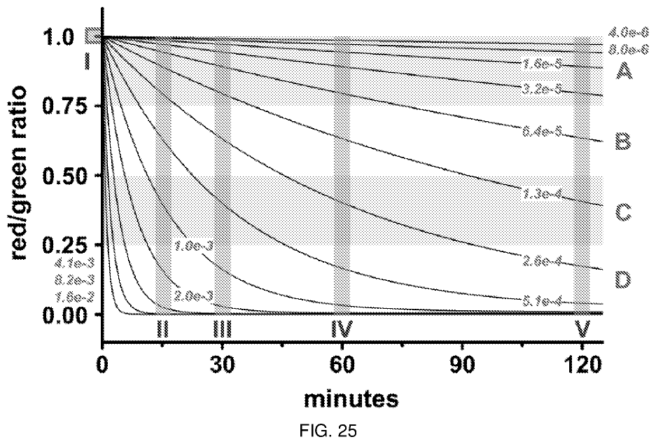

A promising approach to development of a dissociable or cleavable TD for use in 1st SORS has been to selectively bleach the TO1 end of the TD on the premise that the bleached fluorophore is internally cleaved at methine bonds by reactive oxygen species as reported and thereby becomes susceptible to release from the binding pocket. We have thus far achieved partial success (FIG. 24) in that the gFAP/TO1 bleaches efficiently but only about half of the rFAP/MG signal is lost and any released TPD cannot be detected in the supernatant. We are also synthesizing a TD with a disulfide linkage inserted within the PEG linker (see, e.g., FIG. 12). Once the functional SORS reagent is produced, flow cytometry is used to analyze the kinetics of TD cleavage/dissociation initiated release of GFP-rFAPs for each of the 6 testbed systems of known NB/GFP affinities. An important parameter to consider is the rate of TD cleavage because the degree of synchrony determines the time resolution at which dissociation can be followed; which is especially relevant for loose NB/TPD complexes because handling limitations will constrain FACS analysis to time courses of 15 minutes or longer. Release rates may be reduced by running FACS time courses at 4° C. where necessary. Cultures of dummy NB-gFAPs doped at 10-8 and 10-9 levels with the entire testbed suite of NB/TPD complexes to model 1st SORS as applied to an actual library. The quantity of interest is the loss of ratiometric red signal for each NB/TPD complex as a function of time post-cleavage (FIG. 25). This can be manifest in normalized NGS data as the (partial) redistribution of the original number of reads (˜102-104) of a captured NB/TPD complex (bin I-A) to other bins (B-D) in SORS time samples. In a bead and FACS-enriched population of 103-104 distinct NB/TPD complexes, cells bearing bonafide complexes will not be present in equal proportions. The starting population for a SORS assay may well have 100-fold differences in NB representation (e.g. due to a bias in favor of highly expressed TPDs). This disparity may be mimicked by varying the representation within the testbed suite of select tight and loose NB/TPD complexes.

One consideration is the best way to apply SORS if NB/TPD complexes of interest turn out to be loose and sometimes rare because of deficiencies or limitations in NB/TPD epitope recognition. The majority of NB/TPD pairs may fall into a population of loose binders that is difficult to kinetically resolve (off-rate >4×10⁻³ s⁻¹), notably in low complexity libraries. The loose population may be isolated by sorting for very low (<0.05) red/green ratio complexes at T=15 min; this situation can be modeled by sorting out loose NBs with KD≥600 nM from tighter NBs. NGS analysis would identify candidates for pooled affinity maturation.

### Example 6—Use of Different Types of TPD Query Sets to Explore Combinatorial Screens for NBs that Report on Specified Biological Attributes

NGS screening using user-defined sets of TPDs enables one to devise novel combinatorial screening strategies. A query set (or pooled small sets) of up to ˜100 total TPDs may be designed to build focused thematic toolkits by comparison of NBs that differentially bind its members. Based on prototypical TPDs validated above. 3 distinct types of small query sets are tested that address: (i) epitopes on GFP mapped by alanine scanning; (ii) Mutations (variants) of the SARS-CoV-2 spike protein; and (iii) splicing isoforms of mouse α-neurexin. Implementation of TPD query sets relies on validated prototype TPDs that are then systematically varied or mutated based on bioinformatic knowledge. Each TPD within a query set is quantitatively ligated into the NB library vector to create a query member. Initial model query sets may be relatively small and explore biological attributes for which there is supporting information. As above, yeast surface displayed NBs are demonstrated to specifically capture co-expressed secreted cognate TPDs and the binding on a library scale can be analyzed using NGS.

There are diverse established approaches to mapping epitopes, including antibody/antigen co-complex structures (x-ray, NMR), peptide-based and mutagenesis-based. Several mutagenesis-based studies are based on NGS analysis of yeast displayed mutant antigens selected by FACS using purified labeled antibodies. Rather than fine mapping of epitopes, the aim of this query is to demonstrate that in a single screen we can directly isolate NBs from our library that bind different epitopes. Alanine scanning is the most common mutagenesis-based mapping method, and our first query set uses alanine scanning to map select GFP and mCherry epitopes bound by our test NBs.

GFP epitope mapping by NMR shows that our tightest test NBs (LaG16. LaG27) bind different epitopes each covering 53-55 surface amino acids, and the LaG16/GFP and LaM4/mCherry co-complex crystal structures have been determined. This information has been applied to predict, using structure-based software or binding interface database statistics, key contact residues (‘hot-spots’) within the epitopes that contribute significant binding energy. These residues are be mutated to alanine singly or in pairs; the query set exploring these 3 epitopes is limited to about 20 members. The above 3 NBs comprise positive controls for specific binding. We can simultaneously determine if other members of the NBs (LaG29, LaG42, LaG18, LaG11, LaM8), whose binding sites are unknown, bind to these epitopes. Our 2 NB libraries can then be screened using the TD against this query set in 2 stages, first by bead-enrichment then by ratiometric FACS. The FACS output is directly analyzed by NGS; SORS will not be carried out because our test NBs will provide information on the effect of a wide range of NB/TPD affinities on epitope mapping. Because a small query set that relies on bioinformatics-predicted epitope hot-spots is prone to misidentification errors, query members may have to be added that increase the number of alanine substitutions beyond two; but this is believed to be unlikely because the co-complex structures each identify 2-3 side chain contacts that dominate binding energetics (ΔΔG>7 kcal/mol). For more robust data, up to 8 additional NBs whose GFP epitopes have been mapped by NMR can be included.

SARS-CoV-2: Cumulative combinatorial screening is applied to isolate NBs that discriminate between variants of the ACE2 receptor binding domain of the SARS CoV-2 spike protein. There are currently many hundreds of NBs that bind variants of the spike RBD. The NBs derive from immune, naive or synthetic libraries, and have been screened and characterized using several methodologies, including NGS. What is common to these screening technologies is that they are based on purified target antigen, and thus lack the diversity of antigens characteristic of a query set. This initial aim is not to isolate and characterize new and useful NBs as such, but to explore application of a 2-stage cumulative combinatorial screen for the isolation of NBs specific to rapidly diversifying spike RBDs. Emphasis is on simplicity, speed and robustness of screening. Our YSD system is already demonstrated to be able to assay known NBs that bind the SARS-CoV-2 spike RBD.

Using the latest update of the spike protein data-base, a 10-member RBD query set is designed based on the Omicron 21K lineage, 5 of which will represent different BA.1.1 sub-variant sequences. This query set is employed in a first stage screen, using the TD, against both 10⁸ complexity NB libraries. A 10-member RBD query set is then designed that includes as a control 2 first stage queries that generated NBs that specifically bind only that query member 21K lineage and 8-members that represent emerging sub-variants of the Omicron 21L lineage. This query set can be employed in a second stage screen, using the TD, against both 10⁸ complexity NB libraries. If the 2 controls give similar binding distributions in NGS data sets from both stages, the data sets can be combined for purposes of analysis-one can conclude that NBs specific for 21K sub-variants do not cross-bind to 21L sub-variants. It is likely that NBs can be identified that distinguish between the 21K and 21L lineages, whose main-line members differ by 6 aa. However, where sub-variants differ by only 1-5 aa from their parent 21K and 21L lineages; we can choose sub-variants that sample this full range. Because we are combinatorially screening multiple Omicron RBD variants using a synthetic NB library, the recognized RBD epitopes will likely differ substantially from RBD epitopes identified from immune camelid NB libraries. Comparison of NB sets identified from our 2 libraries may be especially informative because in vitro-matured NBs more often use framework 2 residues for binding, and thus may be more frequent in library 1.

α-neurexins (α-Nrxn): Combinatorial multiplexing may be employed to isolate NBs specific for splice isoforms of mouse α-neurexins (α-Nrxn), a highly conserved protein that resides on the presynaptic surface of neural synapse clefts. Alternative splicing (AS) within α-Nrxns leads potentially to thousands of variants. There are 3 α-neurexins that play distinct important roles in the formation and function of synapses: Nrxn1 in excitatory synapses, Nrxn2 in inhibitory synapses, and Nrxn3 in both. Each α-Nrxn includes 6 LNS domains (Laminin-Neurexin-Sex hormone-binding globulin) with divergent sequences; the 3 LNS2 domains are functionally important and are our main focus; including splice isoforms, these domains range in size from 193-208 aa with 77-97% aa identity. Anti-LNS2 NBs are extremely unlikely to cross-bind to other LNS domains (12-31% aa identity). A 7-member TPD query set is constructed, 6 members targeting NBs against the known un-spliced and internally spliced isotypes of LNS2, and 1 member targeting an alternative Nrxn3 translation start that truncates LNS2 to 76 aa. The query set can be screened using the TD against both 10⁸ complexity NB libraries. Select NBs able to differentiate between the 3 neurexins and their splice isoforms can be further characterized for binding affinity using SORS; NB-gFAP fusion proteins will be provided for confirmatory microscopic imaging using neuronal cell lines. We have not yet shown that these neurexin LNS2 domains can be secreted from yeast in our system; however, mouse LNS2 has been secreted from mammalian cells. If secretion is not efficient, we can use neuroligins or another synaptic ectodomain. LNS2 expression and secretion will validated as described above. A difficulty may be that Nrxn1 and Nrxn2 (but not Nrxn3) have 8 aa splice inserts that differ at only 1 aa, so NBs capable of discriminating between them will likely have to bind a discontinuous epitope that includes 1-3 additional flanking residues specific to each unspliced Nrxn. NBs that specifically bind the truncated Nrxn3 LNS2 will depend on the truncated target having a differently folded structure or exposing normally inaccessible residues. In contrast, NBs that specifically bind to the Nrxn2 LNS2 15 aa splice insert or distinguish between the non-splice surfaces of all 3 Nrxns, should be relatively easy to obtain.

This technology is potentially of very high value because it will enable rapid inexpensive isolation of multiple NBs with widely ranging affinities from individual libraries. If desired, these NBs can be used as feedstock for conventional NB workflows, including physical isolation (by synthesis) and characterization, and classical yeast display affinity maturation. While some challenges may arise to implementation using certain TPDs, predominantly in terms of secretion of difficult peptides, and distinguishing different protein isoforms having significant similarity, those challenges are not insurmountable and relate to only a subset of the possible TPDs and NBs that can be readily screened using the described methods and reagents.

Having described this invention, it will be understood to those of ordinary skill in the art that the same can be performed within a wide and equivalent range of conditions, formulations and other parameters without affecting the scope of the invention or any embodiment thereof. References incorporated herein by reference are incorporated for their technical disclosure and only to the extent that they are consistent with the present disclosure.

1. A nucleic acid comprising one or more inducible gene for expressing in a yeast cell:
a first fusion protein comprising amino acid sequences of: an anchor peptide selected for anchoring the first fusion protein in a cell wall of the cell, a nanobody, and an scFv (FAP) for binding a first fluorophore; and
a second fusion protein comprising amino acid sequences of a secretion signal peptide and an scFv (FAP) for binding a second fluorophore,
wherein the first fluorophore increases fluorescent emissions, e.g., at least 1000-fold, at an excitation wavelength when bound by the scFv of the first fusion protein, the second fluorophore increases fluorescent emissions, e.g., at least 1000-fold, at an excitation wavelength when bound by the scFv of the second fusion protein, and the fluorescent emission of the scFv-bound first fluorophore is detectably different from that of the scFv-bound second fluorophore, wherein the nucleic acid further comprises a barcode for identifying a target protein domain (TPD) in the coding sequence of the first fusion protein and wherein the first fusion peptide optionally comprises one or more type IIS restriction endonuclease recognition site-terminated DNA modules.

2. The nucleic acid of claim 1, wherein the second fusion protein comprises a target protein domain.

3. The nucleic acid of claim 1 wherein the first fusion protein comprises amino acid sequences of: in order from an N-terminal to a C-terminal direction, the anchor peptide, the nanobody, and the scFv for binding a first fluorophore; and the second fusion protein comprises amino acid sequences of in order from an N-terminal to C-terminal direction, the secretion signal peptide and the scFv for binding a second fluorophore.

4. The nucleic acid of claim 1, wherein the second fusion protein comprises amino acid sequences of, in order from an N-terminal to C-terminal direction, the secretion signal peptide, a target protein domain, and the scFv for binding a second fluorophore.

5. (canceled)

6. The nucleic acid of claim 1, wherein the one or more genes encoding the first and second fusion proteins are bicistronic.

7. The nucleic acid of claim 6, wherein the genes are under control of a GAL1-10 (GAL1/GAL10) UAS bidirectional promoter.

8. The nucleic acid of any one of claim 1, wherein one of the first and second fluorophore is a triaryl methine dye, such as malachite green or a thiazole orange dye.

9-12. (canceled)

13. The nucleic acid of claim 1, comprising a plurality of type IIS restriction endonuclease recognition site-terminated DNA modules comprising sequences encoding in separate modules:
a first FAP open reading frame (ORF);
an optional TPD ORF;
a secretion leader ORF;
a bidirectional promoter;
a surface display leader ORF, optionally including a TPD barcode;
a NB ORF;
optionally a SAG1 linker ORF; and/or
a second FAP ORF.

14-15. (canceled)

16. The nucleic acid of claim 1, wherein the first FAP and the second FAP have scFv sequences of two of SEQ ID NOS: 3-15.

17. The nucleic acid of claim 1, wherein nanobody comprises the sequence of, or a sequence having at least 90%, at least 95%, or at least 99% sequence identity with the sequence:
(SEQ ID NOS: 23 and 45-48)
QVQLVESGGGLVQAGGSLRLSCAAS(X)ₘRQAPGKERE(X)ₙYADSVKGR
FTISRDNAKNTVYLQMNSLKPEDTAVYYCA(X),YDYWGQGTQVTVS,
excluding (X)ₘ, (X)ₙ, and (X)ₚ, wherein n is 12, m is 12 or 13, and p ranges from 6 to 26, inclusive, and (X)ₘ, (X)ₙ, and (X)ₚ correspond to CRD1, CDR2, and CDR3, respectively of the camelid V_{HH}.

18. The nucleic acid of claim 1, comprising a barcode sequence in the sequence encoding the second fusion protein identifying the TPD of the first fusion protein.

19-20. (canceled)

21. A plurality of nucleic acids as claimed in claim 1, with at least 10⁷ different V_{HH} CDR sequences.

22. A yeast cell comprising the nucleic acid of claim 1.

23. (canceled)

24. A library of yeast cells comprising different colony forming units, with each colony forming unit corresponding to individual nucleic acids of the plurality of nucleic acids of claim 21, optionally comprising S. cerevisiae EBY100 cells transformed with the plurality of nucleic acids.

25. (canceled)

26. A kit comprising the nucleic acid or nucleic acids of claim 1, optionally provided in one or more yeast cells, and a tethered dye any comprising the first fluorophore tethered by a spacer of between 1 nm and 50 nm to the second fluorophore, wherein the first FAP binds the first fluorophore of the tethered dye and the second FAP binds the second fluorophore of the tethered dye, the spacer optionally comprising one or more PEG₁₋₁₀₀ groups

27. A kit comprising two or more part plasmids comprising components for assembly of a nucleic acid of claim 1 using a Golden Gate cloning method, comprising:
a first nucleic acid plasmid, comprising two different Type IIS restriction endonuclease recognition sites; and
one or more additional plasmids comprising sequences flanked by recognition sites of a Type IIS restriction endonuclease, and encoding:
one or more secretion leader peptides;
an E. coli replication origin and antibiotic resistance gene, such as AmpR;
a Gal1-10 UAS bicistronic promoter;
a first fluorogen activating protein;
a second fluorogen activating protein, for activating a fluorogen different from the first fluorogen-activating protein;
a VHH peptide sequence;
a SAG1 linker;
a transcription terminator, such as an ADH1 terminator;
a first selection marker;
a yeast replication origin;
an optional control V_{HH};
a second selection marker; and
an AGA2 anchor sequence and a linker peptide,
each of which being flanked by recognition sites of one of the two different Type IIS restriction endonuclease recognition sites of the first plasmid.

28. The kit of claim 27, wherein the kit comprises five additional plasmids, including:
a second plasmid comprising a sequence encoding the AGA2 anchor sequence and a linker peptide and comprising a barcode nucleic acid sequence;
a third plasmid comprising the sequence encoding the a Gal1-10 UAS bicistronic promoter;
a fourth plasmid comprising a plurality of sequences encoding two or more secretion leaders;
a fifth plasmid comprising a sequence encoding the E. coli replication origin and antibiotic resistance gene, such as AmpR; and
a sixth plasmid comprising sequences encoding a first fluorogen activating protein; a second fluorogen activating protein, for activating a fluorogen different from the first fluorogen-activating protein; a V_{HH} peptide sequence; a SAG1 linker; a transcription terminator, such as an ADH1 terminator; a first selection marker, such as a TRP1 gene; a yeast replication origin; and a second selection marker, such as a KanR gene.

29. (canceled)

30. The kit of claim 28, further comprising at least 10⁹ different versions of the sixth plasmid comprising V_{HH}-encoding sequences encoding at least 10⁹ different V_{HH} peptides.

31-34. (canceled)

35. The kit of claim 26, further comprising a tethered dye comprising the first fluorophore tethered by a spacer of between 1 nm and 50 nm to the second fluorophore, wherein the first FAP binds the first fluorophore of the tethered dye and the second FAP binds the second fluorophore of the tethered dye, the spacer optionally comprising one or more PEG₁₋₁₀₀ groups.

36. (canceled)

37. The kit of any one of claim 26, wherein the first FAP and the second FAP have scFv sequences of two of SEQ ID NOS: 3-15, and one of the first and second fluorophore are malachite green and thiazole orange, and the other of the first and second fluorophore are the other of malachite green and thiazole orange.

38-39. (canceled)

40. The kit of claim 26, wherein the tethered dye has a structure chosen from: 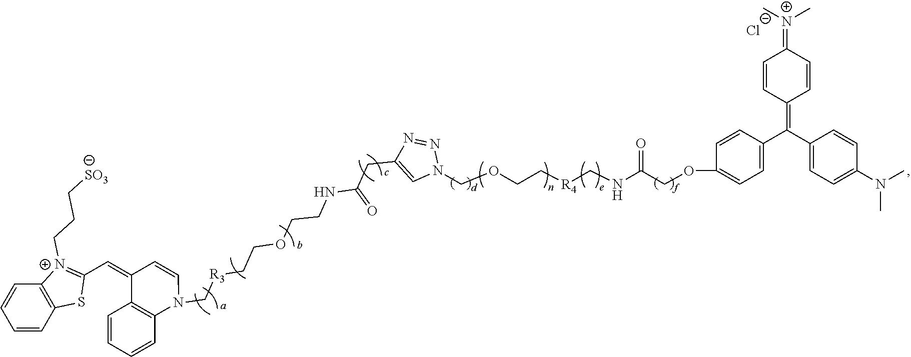
wherein a is an integer from 1 to 6, e.g., from 1 to 4, from 2 to 4, or 3; b is an integer from 1 to 200, e.g., from 1 to 100, from 2 to 50, or 2; c is an integer from 1 to 6, e.g., from 1 to 4, from 1 to 3, or 2; d is an integer from 1 to 6, e.g., from 1 to 4, from 1 to 3, or 2; n is an integer from 1 to 200, e.g., from 1 to 100, from 2 to 100, from 75 to 85, or 80; e is an integer from 1 to 6, e.g., from 1 to 4, from 1 to 3, or 2; f is an integer from 1 to 6, e.g., from 1 to 4, from 2 to 4, or 3; and R₃ and R₄ each independently are 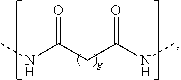
wherein g is an integer from 1 to 6, e.g., from 1 to 4, from 1 to 3, or 2; 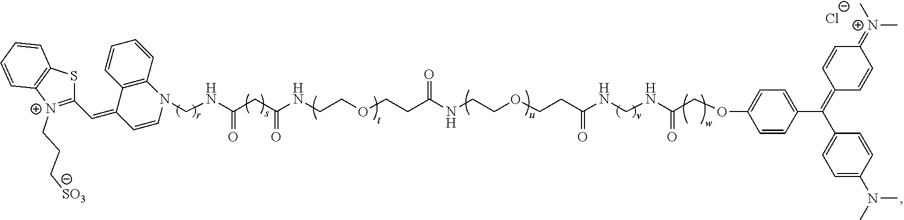
wherein r is an integer from 1 to 6, e.g., from 1 to 4, from 1 to 3, or 3; s is an integer from 1 to 6, e.g., from 1 to 4, from 1 to 3, or 3; t is an integer from 1 to 200, e.g., from 1 to 100, from 2 to 50, or 2; u is an integer from 1 to 200, e.g., from 1 to 100, from 2 to 100, from 75 to 85, or 80; v is an integer from 1 to 6, e.g., from 1 to 4, from 1 to 3, or 2; and w is an integer from 1 to 6, e.g., from 1 to 4, from 1 to 3, or 3; or 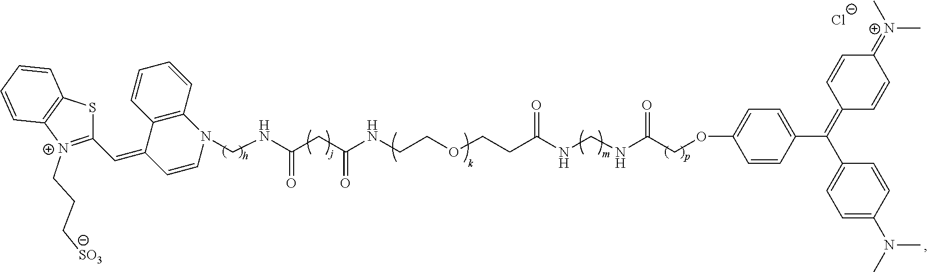
wherein h is an integer from 1 to 6, e.g., from 1 to 4, from 1 to 3, or 3; j is an integer from 1 to 6, e.g., from 1 to 4, from 1 to 3, or 2; k is an integer from 1 to 200, e.g., from 1 to 100, from 2 to 100, from 75 to 85, or 80; m is an integer from 1 to 6, e.g., from 1 to 4, from 1 to 3, or 2; and p is an integer from 1 to 6, e.g., from 1 to 4, from 1 to 3, or 3.

41-42. (canceled)

43. A method of identifying a nanobody that binds a target protein domain, comprising:
growing a yeast cell or cells according to claim 22 in yeast culture medium;
inducing expression of the first fusion protein and the second fusion protein by the yeast cell;
contacting the yeast cell with a tethered dye comprising cognate fluorogen moieties of the first FAP and the second FAP, wherein the cognate fluorogen moieties are separated by a PEG spacer of from 1 nm to 50;
illuminating the yeast cell with light at an excitation wavelength of the cognate fluorogen moieties bound to their cognate FAPs;
and
determining if the yeast cell fluoresces at emission wavelengths of both FAP-bound fluorogen moieties, indicating binding of the first fusion peptide to the second fusion peptide.

44-54. (canceled)
